SEQUENCE LISTING (163 sequences)

SEQ ID NO: 1 - DNA, 5382 bp, Hominidae
  CDS (301)..(2880)
  mat_peptide (364)..(2880)
atcaagtcgcctgccctggcgacactttcgaaggactggagtgggaatcagagcttcacgggttaaaagccgatgtcacatcggccgttcgaaactcctcctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgcccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggcctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggtgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaacaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtatggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatg

SEQ ID NO: 2 - DNA, 25 bp, Hominidae
gagtgggaatcagagcttcacgggt

SEQ ID NO: 3 - DNA, 25 bp, Hominidae
ccacgtcatttacagcatttcaatg

SEQ ID NO: 4 - DNA, 25 bp, Hominidae
actcctccccctgctagaaacctca

SEQ ID NO: 5 - DNA, 25 bp, Hominidae
ttctggcgcttggagcaagccttac

SEQ ID NO: 6 - DNA, 25 bp, Hominidae
cctttctccttttcctctctctcag

SEQ ID NO: 7 - DNA, 25 bp, Hominidae
aaaataaatgcatatcatgcccaaa

SEQ ID NO: 8 - DNA, 25 bp, Hominidae
tgacagttcaatcctgtctcttctg

SEQ ID NO: 9 - DNA, 25 bp, Hominidae
atagcaaaggcagggccacacttac

SEQ ID NO: 10 - DNA, 25 bp, Hominidae
gttgggagacttcacacggtgatgg

SEQ ID NO: 11 - DNA, 25 bp, Hominidae
acttaggcagtggaactcgaaggcc

SEQ ID NO: 12 - DNA, 25 bp, Hominidae
ccccagctgtgggcctgcgacaacg

SEQ ID NO: 13 - DNA, 25 bp, Hominidae
gggggagcccagggacaggtgatag

SEQ ID NO: 14 - DNA, 25 bp, Hominidae
caacacactctgtcctgttttccag

SEQ ID NO: 15 - DNA, 25 bp, Hominidae
ggaaaaccagatggccagcgctcac

SEQ ID NO: 16 - DNA, 25 bp, Hominidae
tccttcctctctctggctctcacag

SEQ ID NO: 17 - DNA, 25 bp, Hominidae
gcaagccgcctgcaccgagactcac

SEQ ID NO: 18 - DNA, 25 bp, Hominidae
agtctgactccctggccctgcgcag

SEQ ID NO: 19 - DNA, 25 bp, Hominidae
agggctcagtccaccggggaatcac

SEQ ID NO: 20 - DNA, 25 bp, Hominidae
ccaagcctctttctctctcttccag

SEQ ID NO: 21 - DNA, 25 bp, Hominidae
ccacccgccgccttcccgtgctcac

SEQ ID NO: 22 - DNA, 25 bp, Hominidae
tccatcgacgggtcccctctgaccc

SEQ ID NO: 23 - DNA, 25 bp, Hominidae
agccctcatctcacctgcgggccaa

SEQ ID NO: 24 - DNA, 25 bp, Hominidae
agatgagggctcctggtgcgatgcc

SEQ ID NO: 25 - DNA, 25 bp, Hominidae
gcccttggtatccgcaacagagaca

SEQ ID NO: 26 - DNA, 25 bp, Hominidae
gatccacagcaacatctactggacc

SEQ ID NO: 27 - DNA, 25 bp, Hominidae
agccctcagcgtcgtggatacgcac

SEQ ID NO: 28 - DNA, 25 bp, Hominidae
cagctattctctgctctcccaccag

SEQ ID NO: 29 - DNA, 25 bp, Hominidae
tgggacggctgtcctcgcaacatac

SEQ ID NO: 30 - DNA, 25 bp, Hominidae
gcacgtgacctctccttatccactt

SEQ ID NO: 31 - DNA, 25 bp, Hominidae
cacctaagtgcttcgatctcgtacg

SEQ ID NO: 32 - DNA, 25 bp, Hominidae
gtcatcttccttgctgcctgtttag

SEQ ID NO: 33 - DNA, 25 bp, Hominidae
gtttccacaaggaggtttcaaggtt

SEQ ID NO: 34 - DNA, 25 bp, Hominidae
cctgactccgcttcttctgccccag

SEQ ID NO: 35 - DNA, 25 bp, Hominidae
cgcagaaacaaggcgtgtgccacac

SEQ ID NO: 36 - DNA, 25 bp, Hominidae
gaagggcctgcaggcacgtggcact

SEQ ID NO: 37 - DNA, 25 bp, Hominidae
gtgtggtggcgggcccagtctttac

SEQ ID NO: 38 - DNA, 25 bp, Hominidae
cctcactcttgcttctctcctgcag

SEQ ID NO: 39 - DNA, 25 bp, Hominidae
cgctgggggaccggcccgcgcttac

SEQ ID NO: 40 - DNA, 25 bp, Hominidae
tgacagagcgtgcctctccctacag

SEQ ID NO: 41 - DNA, 25 bp, Hominidae
gctttctagagagggtcacactcac

SEQ ID NO: 42 - DNA, 25 bp, Hominidae
tccgctgtttaccatttgttggcag

SEQ ID NO: 43 - DNA, 25 bp, Hominidae
aataaaacaaggccggcgaggtctc

SEQ ID NO: 44 - DNA, 18 bp, Hominidae
  exon (1)..(18)
aagtgggtctccgatggc

SEQ ID NO: 45 - DNA, 23 bp, Hominidae
  misc_feature (9)..(9): n is a, c, g, or t
gctgagtgncngnnnngatnagn

SEQ ID NO: 46 - DNA, 18 bp, Hominidae
  exon (1)..(18)
aaatccagggacttcagc

SEQ ID NO: 47 - DNA, 19 bp, Hominidae
  exon (2)..(19)
agtggactgagacaacggc

SEQ ID NO: 48 - DNA, 30 bp, Hominidae
ctgcaggccccaagacgtgctcccaggacg

SEQ ID NO: 49 - DNA, 29 bp, Hominidae
ctgcaggcccaagacgtgctcccaggacg

SEQ ID NO: 50 - DNA, 21 bp, Hominidae
  misc_feature (12)..(12): n is a, c, g, or t
ctgcagccccanaactnnntc

SEQ ID NO: 51 - DNA, 15 bp, Hominidae
  exon (1)..(15)
aagacgtcctcccag

SEQ ID NO: 52 - DNA, 16 bp, Hominidae
aagtacgtcctcccag

SEQ ID NO: 53 - DNA, 16 bp, Hominidae
  misc_feature (5)..(5): n is a, c, g, or t
ctccnaggacgagttt

SEQ ID NO: 54 - DNA, 28 bp, Hominidae
ggtcctgctcaaagcgacggtgctaccc

SEQ ID NO: 55 - DNA, 28 bp, Hominidae
  misc_feature (15)..(15): n is a, c, g, or t
ggtcctgctcaaagngacggtgctaccc

SEQ ID NO: 56 - protein, 8 aa, Hominidae
CHDAILGM

SEQ ID NO: 57 - DNA, 30 bp, Hominidae
  exon (7)..(30)
tttcgctgccacgatgcatctctcggcagt

SEQ ID NO: 58 - DNA, 18 bp, Hominidae
  exon (1)..(18)
gggaagggcatctctcgg

SEQ ID NO: 59 - DNA, 18 bp, Hominidae
  misc_feature (9)..(9): n is a, c, g, or t
gggaagtgnatctctcgg

SEQ ID NO: 60 - DNA, 26 bp, Hominidae
  exon (1)..(18)
cggcagttcgtctgactcagaccggg

SEQ ID NO: 61 - DNA, 20 bp, Hominidae
  exon (1)..(18)
tgtgactccagaccgggact

SEQ ID NO: 62 - DNA, 21 bp, Hominidae
  exon (1)..(21)
gaccgggaccgcttggacgga

SEQ ID NO: 63 - DNA, 21 bp, Hominidae
gaccgggaccgcttggacggc

SEQ ID NO: 64 - DNA, 27 bp, Hominidae
  misc_feature (19)..(19): n is a, c, g, or t
tcagaccgggactgcttgnacggctca

SEQ ID NO: 65 - protein, 10 aa, Hominidae
FQGDSSPCSA

SEQ ID NO: 66 - protein, 4 aa, Hominidae
APAR

SEQ ID NO: 67 - DNA, 29 bp, Hominidae
  exon (1)..(27)
ttccaaggggacagtagcccctgctcggt

SEQ ID NO: 68 - DNA, 22 bp, Hominidae
  exon (1)..(21)
ttccaaggggcccctgctcggt

SEQ ID NO: 69 - DNA, 22 bp, Hominidae
  misc_feature (11)..(11): n is a, c, g, or t
ttccaaggggncnntgntcngt

SEQ ID NO: 70 - DNA, 37 bp, Hominidae
aaatctgacgaggaaaactgcggtatgggcggggcca

SEQ ID NO: 71 - DNA, 49 bp, Hominidae
aaatctgacaggacaaatctgacgaggaaaactgcggtatgggcggggc

SEQ ID NO: 72 - DNA, 25 bp, Hominidae
  misc_feature (10)..(10): n is a, c, g, or t
aaatctgacnaggcctatgnanact

SEQ ID NO: 73 - DNA, 22 bp, Hominidae
tgcgaagatcggatgagtggcc

SEQ ID NO: 74 - protein, 12 aa, Hominidae
DGDVAGRGNEKK

SEQ ID NO: 75 - DNA, 36 bp, Hominidae
  exon (1)..(36)
ctgggcgacgttgctggcgagggaaatgagaagaag

SEQ ID NO: 76 - DNA, 15 bp, Hominidae
  exon (1)..(15)
ctgggcgagaagaag

SEQ ID NO: 77 - protein, 5 aa, Hominidae
DGEKK

SEQ ID NO: 78 - DNA, 34 bp, Hominidae
  misc_feature (18)..(18): n is a, c, g, or t
cctgcagctctgggcganaagaagcccagtagcg

SEQ ID NO: 79 - DNA, 15 bp, Hominidae
  exon (1)..(15)
tctgacaaggaaaac

SEQ ID NO: 80 - DNA, 15 bp, Hominidae
  exon (1)..(15)
tctgaccaggaaaac

SEQ ID NO: 81 - DNA, 18 bp, Hominidae
gacgcgtccctggttgct

SEQ ID NO: 82 - DNA, 18 bp, Hominidae
acgcgtccctggtttgct

SEQ ID NO: 83 - DNA, 24 bp, Hominidae
  misc_feature (4)..(4): n is a, c, g, or t
gccnggnccngntcccgggttgct

SEQ ID NO: 84 - DNA, 20 bp, Hominidae
tccctggttgcttacgaacc

SEQ ID NO: 85 - DNA, 19 bp, Hominidae
tcccgggttcttacgaacc

SEQ ID NO: 86 - DNA, 18 bp, Hominidae
ccgatgctcacggacacg

SEQ ID NO: 87 - DNA, 18 bp, Hominidae
ccgatgctctcggacacg

SEQ ID NO: 88 - DNA, 22 bp, Hominidae
  exon (1)..(21)
ggctacgagcgcctgtgccccg

SEQ ID NO: 89 - DNA, 22 bp, Hominidae
  misc_feature (9)..(9): n is a, c, g, or t
ggctacgancgcctgtgccccg

SEQ ID NO: 90 - DNA, 29 bp, Hominidae
gtgaacctgtgtgaggaaggcttccagct

SEQ ID NO: 91 - DNA, 28 bp, Hominidae
  misc_feature (10)..(10): n is a, c, g, or t
gtgaacctgnangntgnntncnagnncc

SEQ ID NO: 92 - DNA, 24 bp, Hominidae
ccagattatcgatgagtgtcagga

SEQ ID NO: 93 - DNA, 24 bp, Hominidae
ggtctaatagctactcacagtcct

SEQ ID NO: 94 - DNA, 18 bp, Hominidae
  exon (1)..(18)
atcgattagtgtcaggat

SEQ ID NO: 95 - DNA, 18 bp, Hominidae
  exon (1)..(18)
gatgagcgtcaggatccc

SEQ ID NO: 96 - DNA, 25 bp, Hominidae
  misc_feature (14)..(14): n is a, c, g, or t
ggtggctacaagtnccagtgtgagg

SEQ ID NO: 97 - protein, 9 aa, Hominidae
VNLGYDKCQ

SEQ ID NO: 98 - DNA, 31 bp, Hominidae
  exon (3)..(29)
gcgtgaacctgggcactgacaagtgccagtg

SEQ ID NO: 99 - DNA, 31 bp, Hominidae
gcgtgaacctggcactgaacaagtgccagtg

SEQ ID NO: 100 - DNA, 21 bp, Hominidae
ttcacggtcagactccttccg

SEQ ID NO: 101 - DNA, 21 bp, Hominidae
  misc_feature (11)..(11): n is a, c, g, or t
ttcacggtcanactccttccg

SEQ ID NO: 102 - DNA, 23 bp, Hominidae
gctccccggacccccaggctcca

SEQ ID NO: 103 - DNA, 23 bp, Hominidae
cgaggggcctgggggtccgaggt

SEQ ID NO: 104 - DNA, 23 bp, Hominidae
  misc_feature (8)..(8): n is a, c, g, or t
cgaggggnctgggggtccgaggt

SEQ ID NO: 105 - DNA, 24 bp, Hominidae
  exon (1)..(24)
tacctcttcctcaccaaccggcac

SEQ ID NO: 106 - protein, 7 aa, Hominidae
SIAYLFT

SEQ ID NO: 107 - DNA, 29 bp, Hominidae
  exon (6)..(29)
caggctccatcgccatcctcttcttcacc

SEQ ID NO: 108 - DNA, 29 bp, Hominidae
gtccgaggtagcggatggagaagaagtgg

SEQ ID NO: 109 - DNA, 26 bp, Hominidae
gtccgaggtagcggatggagaagtgg

SEQ ID NO: 110 - DNA, 18 bp, Hominidae
  misc_feature (7)..(8): n is a, c, g, or t
tagcggnngnagaagtgg

SEQ ID NO: 111 - DNA, 16 bp, Hominidae
  exon (1)..(15)
ttcaccaactggcacg

SEQ ID NO: 112 - DNA, 17 bp, Hominidae
  exon (3)..(17)
accggcacaaggtcagg

SEQ ID NO: 113 - DNA, 24 bp, Hominidae
  exon (1)..(24)
atgacgctggaccggaccggagcg

SEQ ID NO: 114 - DNA, 15 bp, Hominidae
  misc_feature (8)..(8): n is a, c, g, or t
accagccncatcccc

SEQ ID NO: 115 - DNA, 24 bp, Hominidae
  exon (1)..(24)
aacgtggtcactctggacacggag

SEQ ID NO: 116 - DNA, 18 bp, Hominidae
  misc_feature (10)..(10): n is a, c, g, or t
gtcgctctgnacacggag

SEQ ID NO: 117 - DNA, 36 bp, Hominidae
  exon (13)..(30)
cccaccagcttcatgtactgaactgactggggaact

SEQ ID NO: 118 - DNA, 24 bp, Hominidae
  misc_feature (14)..(14): n is a, c, g, or t
cttcatgtactganttaatnggga

SEQ ID NO: 119 - DNA, 29 bp, Hominidae
  exon (3)..(26)
ttcagtggcccaatggcatcaccctagag

SEQ ID NO: 120 - DNA, 27 bp, Hominidae
  exon (3)..(26)
ttcagtggccccaatggcatcacccta

SEQ ID NO: 121 - DNA, 28 bp, Hominidae
  misc_feature (12)..(12): n is a, c, g, or t
ttcagtggcccnanggnnttanccttag

SEQ ID NO: 122 - DNA, 19 bp, Hominidae
ggtgtggctggtgactgaa

SEQ ID NO: 123 - DNA, 28 bp, Hominidae
  misc_feature (9)..(9): n is a, c, g, or t
ggtgtgganatcnactgnntgnnnacng

SEQ ID NO: 124 - DNA, 20 bp, Hominidae
ggtgtggacactactcgctg

SEQ ID NO: 125 - DNA, 18 bp, Hominidae
  exon (1)..(12)
ggcatcaccctaggtatg

SEQ ID NO: 126 - DNA, 18 bp, Hominidae
  misc_feature (10)..(10): n is a, c, g, or t
ggcatcaccntaggtatg

SEQ ID NO: 127 - DNA, 15 bp, Hominidae
cccaggtatgttcgc

SEQ ID NO: 128 - DNA, 16 bp, Hominidae
ccctaggtatgttcgc

SEQ ID NO: 129 - DNA, 15 bp, Hominidae
cccaggtatgttcgc

SEQ ID NO: 130 - DNA, 16 bp, Hominidae
ccctaggtatgttcgc

SEQ ID NO: 131 - DNA, 15 bp, Hominidae
  exon (1)..(15)
gatgtcaacgggggc

SEQ ID NO: 132 - protein, 7 aa, Hominidae
DVNGPQP

SEQ ID NO: 133 - DNA, 22 bp, Hominidae
  exon (1)..(21)
gatgtcaacgggggcaaccgga

SEQ ID NO: 134 - DNA, 22 bp, Hominidae
  exon (1)..(21)
gatgtcaacggggggcaaccgg

SEQ ID NO: 135 - DNA, 22 bp, Hominidae
  misc_feature (15)..(16): n is a, c, g, or t
gatgtcaacgggggnnanccgn

SEQ ID NO: 136 - DNA, 22 bp, Hominidae
  misc_feature (9)..(9): n is a, c, g, or t
aggctggcncacnccttctcct

SEQ ID NO: 137 - DNA, 35 bp, Hominidae
  exon (1)..(33)
ttttggacagatatcaacgaagccattttcagtgc

SEQ ID NO: 138 - DNA, 23 bp, Hominidae
  misc_feature (17)..(17): n is a, c, g, or t
ttttggacagatatcancaanga

SEQ ID NO: 139 - DNA, 18 bp, Hominidae
  exon (3)..(17)
gtgccaactgcctcacag

SEQ ID NO: 140 - DNA, 21 bp, Hominidae
  exon (3)..(20)
tggttctctccacaacctcac

SEQ ID NO: 141 - DNA, 17 bp, Hominidae
  misc_feature (5)..(5): n is a, c, g, or t
tggtnctcttcanaaac

SEQ ID NO: 142 - DNA, 22 bp, Hominidae
  exon (2)..(22)
ctgccagttatctgtgcctccc

SEQ ID NO: 143 - protein, 4 aa, Hominidae
CLTK

SEQ ID NO: 144 - DNA, 19 bp, Hominidae
  exon (2)..(10)
ctgcctcacaagtgtggca

SEQ ID NO: 145 - DNA, 34 bp, Hominidae; identical to SEQ ID NO: 78
  misc_feature (18)..(18): n is a, c, g, or t

SEQ ID NO: 146 - protein, 9 aa, Hominidae
LLVFLCLLS

SEQ ID NO: 147 - DNA, 36 bp, Hominidae
  exon (6)..(35)
cagtgctcctcgtcttcctttgcctgggggtcttcc

SEQ ID NO: 148 - DNA, 36 bp, Hominidae
  exon (6)..(35)
cagtgctcctcgtcttcctttgcctggggggtcttc

SEQ ID NO: 149 - DNA, 36 bp, Hominidae
gtcacgaggagcagaaggaaacggacccccagaagg

SEQ ID NO: 150 - DNA, 36 bp, Hominidae
tcacgaggagcagaaggaaacggaccccccagaagg

SEQ ID NO: 151 - DNA, 27 bp, Hominidae
  misc_feature (3)..(3): n is a, c, g, or t
acnaganggaacngaacccccagaagg

SEQ ID NO: 152 - DNA, 21 bp, Hominidae
tagccgatgctcacggacacg

SEQ ID NO: 153 - DNA, 19 bp, Hominidae
  exon (2)..(19)
gggcgacagatgcgaaaga

SEQ ID NO: 154 - DNA, 18 bp, Hominidae
  misc_feature (9)..(9): n is a, c, g, or t
atccactcnagctggcgc

SEQ ID NO: 155 - DNA, 17 bp, Hominidae
  exon (1)..(15)
aacaacggtggctgttc

SEQ ID NO: 156 - DNA, 21 bp, Hominidae
  exon (1)..(21)
gaccttaagattggctacgag

SEQ ID NO: 157 - DNA, 21 bp, Hominidae
  misc_feature (21)..(21): n is a, c, g, or t
gaccttaagattggctacgan

SEQ ID NO: 158 - DNA, 26 bp, Hominidae
  exon (7)..(24)
cgaggctccatcgcctacctcttctt

SEQ ID NO: 159 - DNA, 26 bp, Hominidae
  exon (7)..(24)
cgaggctccattgcctacctcttctt

SEQ ID NO: 160 - DNA, 25 bp, Hominidae
  exon (6)..(23)
caggctccattgcctacctcttctt

SEQ ID NO: 161 - DNA, 16 bp, Hominidae
  exon (1)..(15)
ggccgcctttactggg

SEQ ID NO: 162 - DNA, 16 bp, Hominidae
  misc_feature (6)..(6): n is a, c, g, or t
gtcaangggggcaacc

SEQ ID NO: 163 - DNA, 12 bp, Hominidae
  misc_feature (6)..(6): n is a, c, g, or t
atggtnctcttc

(19) United States
(12) Patent Application Publication
(10) Pub. No.: US 2004/0101863 A1
(43) Pub. Date: May 27, 2004

(54) Method of detecting lipid metabolic errors

(76) Inventors: Hiroaki Hattori, Saitama (JP); Masahiro Tsuji, Hokkaido (JP); Tomoo Okada, Tokyo (JP); Makoto Nagano, Saitama (JP); Tohru Egashira, Saitama (JP); Mitsuaki Ishihara, Saitama (JP); Tadao Iwasaki, Saitama (JP)

Correspondence Address: SUGHRUE MION, PLLC, 2100 PENNSYLVANIA AVENUE, N.W., SUITE 800, WASHINGTON, DC, 20037, US

(21) Appl. No.: 10/333,068

(22) PCT Filed: Jul. 17, 2001
(86) PCT No.: PCT/JP01/06153

(30) Foreign Application Priority Data
Jul. 18, 2000 (JP) 2000-218039

(51) Int. Cl. C12Q 1/68

(52) U.S. Cl. 435/6

(57) ABSTRACT

The intended object of the present invention is to provide a more widely applicable means of diagnosing familial hyperlipemia with certainty. It has been found that this object can be attained by providing a method of detecting abnormality of lipid metabolism, wherein risk factors concerning abnormalities of lipid metabolism are detected on the basis of the correlation of 65 specific types of LDL receptor gene mutations with the abnormalities of lipid metabolism, as well as a method of detecting disease(s) by detecting risk factors for arteriosclerosis and/or ischemic heart diseases by employing as indices the abnormalities of lipid metabolism thus detected.

### TECHNICAL FIELD

The present invention relates to a method of detecting diseases, and more particularly to a method of detecting abnormalities or errors of lipid metabolism through employment of certain gene mutations as indices.

### BACKGROUND ART

Errors of metabolism of serum lipids cause abnormal serum lipid levels, and possibly lead to undesirable pathological conditions. In particular, arteriosclerosis and ischemic heart diseases are typical diseases caused by abnormalities of serum lipid metabolism. However, a wide diversity of clinical symptoms are caused by abnormal metabolism of serum lipids causative of these specific pathological conditions, and thus their diagnoses are also diversified.

Of serum lipid metabolic disorders or errors, familial hypercholesterolemia (FH) is known to be caused by qualitative or quantitative abnormalities of the low-density-lipoprotein (LDL) receptor (R). FH presents high cholesterol levels—particularly high LDL cholesterol levels—in blood, and is a typical autosomal dominant hereditary disease characterized by early coronary heart disease. In other words, familial hypercholesterolemia is frequently responsible for coronary heart diseases, inter alia, those occurring during childhood.

FH manifests coronary heart disease in males in their 20 s, and in post-menopausal women in their 40 s. Homozygous familial hypercholesterolemia patients, who inherit two abnormal alleles from their parents, are rare (about 1 in one million individuals) and show very high serum cholesterol levels of about 1,000 mg/dL. Diagnosis of homozygous familial hypercholesterolemia is not difficult. In contrast, heterozygous familial hypercholesterolemia patients, who inherit one abnormal allele from their parents, are encountered at a frequency of about 1 in 500 individuals. In Japan, the number of patients suffering heterozygous familial hypercholesterolemia is estimated to be about 250,000. However, depending on the site of the LDL receptor gene at which abnormality exists, heterozygous FH exhibits different activities of LDL receptor protein in terms of the protein's ability to bind a ligand LDL and take it into cells. As a result of these differences in activity, heterozygous FH patients are known to follow a variety of courses leading to the development of arteriosclerosis. As has also been known, different types of abnormality lead to arteriosclerosis of different severity levels. In general, therapy of familial hypercholesterolemia is preferably initiated as early as possible in the juvenile stage by administering appropriate drugs, such as HMG-CoA reductase inhibitors, agents of fibrate series, and antioxidants, so as to prevent the onset of coronary atherosclerosis or arteriosclerosis, which aggravates with age. However, difficulty in diagnosis of heterozygous FH in childhood tends to impede early detection and treatment of FH. Precise diagnosis of FH would also be effective for enabling early diagnosis of potential FH in other family members; FH is dominantly inherited, and if FH is found in a certain family member, other family members can be predicted to suffer FH.

As mentioned above, current diagnosis indices for familial hypercholesterolemia include, among others, high blood cholesterol level, presence or absence of tendonous xanthoma, early coronary events, and familial history. All indices other than familial history and high blood cholesterol level are concerned with conditions of hypercholesterolemia of considerably advanced stage. Particularly for successful diagnosis of FH heterozygotes during childhood, in which elevation in serum cholesterol is not necessarily significant, use of only these diagnosis indices is insufficient.

In addition to the above-mentioned diagnosis indices, a new means for determining familial hypercholesterolemia through measuring LDL receptor activity has presently been suggested, in which tissue or cells collected from a living body are cultured to thereby induce expression of the LDL receptors of the cells, and binding, uptake, and catabolism of radioisotope-labeled LDL are measured for comparison with the case of cells from healthy subjects. Another approach is direct determination of the presence or absence of mutations of LDL receptors through PCR using genomic DNA obtained from patients. However, the former approach involves the problem that it does not lend itself to general practice, as it requires a special facility for handling a radioisotope, as well as skill to perform intricate manipulation procedure. Also, the latter approach has the drawback in that, since more than about 350 types of LDL receptor gene mutations have so far been identified, determination of respective mutations one by one through, for example, PCR raises problems in terms of time and cost. Currently, in order to render ensured diagnosis of FH, these approaches, which can be employed only in limited areas, are the only means to be employed.

As described above, from the viewpoint of prevention of coronary diseases and arteriosclerosis, FH must be detected as early as possible, before the typical clinical findings of FH have become clear in the patient. Thus, provision of a widely-applicable technique that enables an established diagnosis of FH would be considered a great contribution to early detection of FH.

Accordingly, an object of the present invention is to provide useful means for rendering an established diagnosis of FH in broader clinical situations.

### DISCLOSURE OF THE INVENTION

The present inventors have noted that familial hypercholesterolemia, known to present elevated blood LDL level, is primarily caused by abnormality occurring in the LDL receptor gene; a blueprint of LDL receptor protein expressed on the cell membrane of hepatocytes or peripheral cells so as to intake LDL. Such abnormality produces an LDL receptor which is abnormal in quality; i.e., incapability to bind blood LDL, or in quantity; i.e., incapability to express sufficient amount of blood LDL receptor protein on the membrane. The inventors have also noted that the causes bringing about these qualitative and quantitative abnormalities reside in abnormalities in the LDL receptor gene itself, and have continued careful studies. As a result, the present inventors have found that the above-mentioned object of the invention can be attained through identification of the mentioned abnormalities responsible for Japanese FH, along with provision of a method for detecting relevant abnormal genes.

Accordingly, the present invention provides a method of detecting abnormality of lipid metabolism (hereinafter may be referred to as the present abnormality detection method), in which a risk factor concerning abnormality of lipid metabolism is detected through correlating one or more gene mutations selected from the group consisting of the below-described 1 through 65 gene mutations with abnormality in lipid metabolism. Moreover, the present invention provides a method of detecting a disease, by detecting a risk factor for an arteriosclerosis and/or ischemic heart disease employing, as an index, abnormality of lipid metabolism detected through the present abnormality detection method (hereinafter may be referred to as the present disease detection method, and the present abnormality detection method and the present disease detection method may be collectively referred to as the present detection method):

1. a mutation of a low-density lipoprotein receptor gene coding for low-density lipoprotein receptor protein, the mutation occurring at a site coding for amino acid residue 25, cysteine, of the low-density lipoprotein receptor protein;

2. a deletion mutation occurring in nucleotides 156 to 160 of the low-density lipoprotein receptor gene;

3. a mutation of the low-density lipoprotein receptor gene occurring at a site coding for amino acid residue 50, glycine, of the low-density lipoprotein receptor protein encoded by the gene;

4. a mutation of the low-density lipoprotein receptor gene occurring at a site coding for amino acid residue 74, cysteine, of the low-density lipoprotein receptor protein encoded by the gene;

5. a deletion mutation occurring in nucleotide 314, cytosine, of the low-density lipoprotein receptor gene;

6. a mutation of the low-density lipoprotein receptor gene occurring at a site coding for amino acid residue 81, glutamine, of the low-density lipoprotein receptor protein encoded by the gene;

7. a mutation of the low-density lipoprotein receptor gene occurring at a site coding for amino acid residue 88, cysteine, of the low-density lipoprotein receptor protein encoded by the gene;

8. a mutation of the low-density lipoprotein receptor gene occurring at a site coding for amino acid residue 90, glutamine, of the low-density lipoprotein receptor protein encoded by the gene;

9. a mutation of the low-density lipoprotein receptor gene occurring at a site coding for amino acid residue 94, arginine, of the low-density lipoprotein receptor protein encoded by the gene;

10. a deletion mutation occurring in nucleotides 355 to 361, of the low-density lipoprotein receptor gene;

11. a mutation of the low-density lipoprotein receptor gene occurring at a site coding for amino acid residue 100, cysteine, of the low-density lipoprotein receptor protein encoded by the gene;

12. a deletion mutation occurring in nucleotides 382 and 383, of the low-density lipoprotein receptor gene;

13. an insertion mutation occurring at a position of nucleotide 390 of the low-density lipoprotein receptor gene;

14. a mutation of the low-density lipoprotein receptor gene occurring at a site coding for amino acid residue 113, cysteine, of the low-density lipoprotein receptor protein encoded by the gene;

15. a mutation of the low-density lipoprotein receptor gene occurring at a site coding for amino acid residue 115, aspartic acid, of the low-density lipoprotein receptor protein encoded by the gene;

16. a mutation of the low-density lipoprotein receptor gene occurring at a site coding for amino acid residue 119, glutamic acid, of the low-density lipoprotein receptor protein encoded by the gene;

17. a deletion mutation occurring in nucleotides 578 to 584, of the low-density lipoprotein receptor gene;

18. an insertion mutation occurring at a position of nucleotide 682 of the low-density lipoprotein receptor gene;

19. a deletion mutation occurring in nucleotides 526 to 529, of the low-density lipoprotein receptor gene;

20. an insertion mutation occurring at a position of nucleotide 661 of the low-density lipoprotein receptor gene;

21. a mutation of the low-density lipoprotein receptor gene occurring at a site coding for amino acid residue 207, glutamic acid, of the low-density lipoprotein receptor protein encoded by the gene;

22. an insertion mutation occurring at a position of nucleotide 944 of the low-density lipoprotein receptor gene;

23. a deletion mutation occurring in nucleotide 948, cytosine, of the low-density lipoprotein receptor gene;

24. a mutation of the low-density lipoprotein receptor gene occurring at a site coding for amino acid residue 317, cysteine, of the low-density lipoprotein receptor protein encoded by the gene;

25. a deletion mutation occurring in nucleotides 1114 to 1134, of the low-density lipoprotein receptor gene;

26. an insertion mutation occurring at a position of nucleotide 1062 of the low-density lipoprotein receptor gene;

27. a mutation of the low-density lipoprotein receptor gene occurring at a site coding for amino acid residue 336, glutamic acid, of the low-density lipoprotein receptor protein encoded by the gene;

28. a mutation of the low-density lipoprotein receptor gene occurring at a site coding for amino acid residue 337, cysteine, of the low-density lipoprotein receptor protein encoded by the gene;

29. a mutation of the low-density lipoprotein receptor gene occurring at a site coding for amino acid residue 356, cysteine, of the low-density lipoprotein receptor protein encoded by the gene;

30. a mutation of the low-density lipoprotein receptor gene occurring at a site coding for amino acid residues 351 to 354, glutamic acid—glycine—glycine—tyrosine, of the low-density lipoprotein receptor protein encoded by the gene;

31. a mutation of the low-density lipoprotein receptor gene occurring at a site coding for amino acid residue 358, cysteine, of the low-density lipoprotein receptor protein encoded by the gene;

32. a mutation occurring at nucleotide −10 (guanine) in the 5′-end-side acceptor region in intron 8 of the low-density lipoprotein receptor gene;

33. a mutation of the low-density lipoprotein receptor gene occurring at a site coding for amino acid residue 382, phenylalanine, of the low-density lipoprotein receptor protein encoded by the gene;

34. a mutation occurring in nucleotide 1599, of the low-density lipoprotein receptor gene;

35. a deletion mutation occurring in nucleotides 1202 to 1204, of the low-density lipoprotein receptor gene;

36. a mutation of the low-density lipoprotein receptor gene occurring at a site coding for amino acid residue 385, arginine, of the low-density lipoprotein receptor protein encoded by the gene;

37. a mutation of the low-density lipoprotein receptor gene occurring at a site coding for amino acid residue 387, glutamic acid, of the low-density lipoprotein receptor protein encoded by the gene;

38. an insertion mutation occurring at a position of nucleotide 1242 of the low-density lipoprotein receptor gene;

39. a mutation of the low-density lipoprotein receptor gene occurring at a site coding for amino acid residue 401, leucine, of the low-density lipoprotein receptor protein encoded by the gene;

40. a mutation of the low-density lipoprotein receptor gene occurring at a site coding for amino acid residue 410, alanine, of the low-density lipoprotein receptor protein encoded by the gene;

41. a mutation of the low-density lipoprotein receptor gene occurring at a site coding for amino acid residue 412, aspartic acid, of the low-density lipoprotein receptor protein encoded by the gene;

42. a mutation of the low-density lipoprotein receptor gene occurring at a site coding for amino acid residue 512, tryptophan, of the low-density lipoprotein receptor protein encoded by the gene;

43. a deletion mutation occurring in nucleotides 1652 to 1662, of the low-density lipoprotein receptor gene;

44. a deletion mutation occurring in nucleotide 1655, thymine, of the low-density lipoprotein receptor gene;

45. an insertion mutation occurring at a position of nucleotide 1687 of the low-density lipoprotein receptor gene;

46. a mutation of the low-density lipoprotein receptor gene occurring at a site coding for amino acid residue 547, leucine, of the low-density lipoprotein receptor protein encoded by the gene;

47. a mutation from guanine to another base, occurring at nucleotide +1 (guanine) in a splice donor site of intron 11 starting from the nucleotide that is one base downstream the nucleotide 1705 of the low-density lipoprotein receptor protein gene;

48. a mutation from thymine to another base, occurring at nucleotide +2 (thymine) in a splice donor site of intron 12 starting from the nucleotide that is one base downstream the nucleotide 1845 of the low-density lipoprotein receptor protein gene;

49. a mutation of the low-density lipoprotein receptor gene occurring at a site coding for amino acid residue 556, tryptophan, of the low-density lipoprotein receptor protein encoded by the gene;

50. a mutation of the low-density lipoprotein receptor gene occurring at a site coding for amino acid residue 570, asparagine, of the low-density lipoprotein receptor protein encoded by the gene;

51. an insertion mutation occurring at a position of nucleotide 1779 of the low-density lipoprotein receptor gene;

52. a mutation of the low-density lipoprotein receptor gene occurring at a site coding for amino acid residue 587, proline, of the low-density lipoprotein receptor protein encoded by the gene;

53. a mutation of the low-density lipoprotein receptor gene occurring at a site coding for amino acid residue 591, alanine, of the low-density lipoprotein receptor protein encoded by the gene;

54. a deletion mutation occurring in nucleotides 1870 to 1872, of the low-density lipoprotein receptor gene;

55. a mutation of the low-density lipoprotein receptor gene occurring at a site coding for amino acid residue 612, arginine, of the low-density lipoprotein receptor protein encoded by the gene;

56. a deletion mutation occurring in nucleotide 1963, of the low-density lipoprotein receptor gene;

57. an insertion mutation occurring at a position of nucleotide 2035 of the low-density lipoprotein receptor gene;

58. a mutation of the low-density lipoprotein receptor gene occurring at a site coding for amino acid residue 664, proline, of the low-density lipoprotein receptor protein encoded by the gene;

59. a mutation of the low-density lipoprotein receptor gene occurring at a site coding for amino acid residue 693, glutamic acid, of the low-density lipoprotein receptor protein encoded by the gene;

60. a deletion mutation occurring in nucleotides 2320 to 2340, of the low-density lipoprotein receptor gene;

61. a mutation of the low-density lipoprotein receptor gene occurring at a site coding for amino acid residue 779, valine, of the low-density lipoprotein receptor protein encoded by the gene;

62. a mutation of the low-density lipoprotein receptor gene occurring at a site coding for amino acid residue 790, lysine, of the low-density lipoprotein receptor protein encoded by the gene;

63. an insertion mutation occurring at a position of nucleotide 2412 of the low-density lipoprotein receptor gene;

64. a mutation of the low-density lipoprotein receptor gene occurring at a site coding for amino acid residue 829, alanine, of the low-density lipoprotein receptor protein encoded by the gene; and

65. a mutation of the low-density lipoprotein receptor gene occurring at a site coding for amino acid residue 316, glutamic acid, of the low-density lipoprotein receptor protein encoded by the gene.

As used herein, amino acid residues are expressed on the basis of the three-letter code system or the one-letter code system. For the sake of convenience, the following may be referred to: alanine [Ala (according to the three-letter code system, the same applies hereunder), A (according to the one-letter code system, the same applies hereunder)], valine [Val, V], leucine [Leu, L], isoleucine [Ile, I], proline [Pro, P], phenylalanine [Phe, F] tryptophan [Trp, W], methionine [Met, M], glycine [Gly, G], serine [Ser, S], threonine [Thr, T], cysteine [Cys, C], glutamine [Gln, Q], asparagine [Asn, N], tyrosine [Tyr, Y], lysine [Lys, K], arginine [Arg, R], histidine [His, H], aspartic acid [Asp, D], glutamic acid [Glu, E].

As used herein, “A100V,” for example, refers to a mutation of the 100th amino acid residue, alanine, in the sequence of native amino acid, such that the residue has been substituted by valine.

### BRIEF DESCRIPTION OF THE DRAWINGS

FIG. 1 shows electrophoresis patterns showing abnormalities in exons 2, 3, 4, and 7 of the LDL receptor gene;

FIG. 2 shows electrophoresis patterns showing abnormalities in exons 8, 9, 11, and 12 of the LDL-R gene;

FIG. 3 shows electrophoresis patterns showing abnormalities in exons 13, 14, 16, 17, and 18 of the LDL-R gene;

FIG. 4 shows a mutation of the LDL-R gene occurring at a site coding for amino acid residue 25, cysteine, of the LDL-R protein encoded by the gene;

FIG. 5 shows a deletion mutation occurring in nucleotides 156 to 160 of the LDL-R gene;

FIG. 6 shows a mutation of the LDL-R gene occurring at a site coding for amino acid residue 50, glycine, of the LDL-R protein encoded by the gene;

FIG. 7 shows a mutation of the LDL-R gene occurring at a site coding for amino acid residue 74, cysteine, of the LDL-R protein encoded by the gene;

FIG. 8 shows another mutation of the LDL-R gene occurring at a site coding for amino acid residue 74; cysteine, of the LDL-R protein encoded by the gene.

FIG. 9 shows a deletion mutation occurring in nucleotide 314, cytosine, of the LDL-R gene;

FIG. 10 shows a mutation of the LDL-R gene occurring at a site coding for amino acid residue 81, glutamine, of the LDL-R protein encoded by the gene;

FIG. 11 shows a mutation of the LDL-R gene occurring at a site coding for amino acid residue 88, cysteine, of the LDL-R protein encoded by the gene;

FIG. 12 shows a mutation of the LDL-R gene occurring at a site coding for amino acid residue 90, glutamine, of the LDL-R protein encoded by the gene;

FIG. 13 shows a mutation of the LDL-R gene occurring at a site coding for amino acid residue 94, arginine, of the LDL-R protein encoded by the gene;

FIG. 14 shows a deletion mutation occurring in nucleotides 355 to 361, of the LDL-R gene;

FIG. 15 shows a mutation of the LDL-R gene occurring at a site coding for amino acid residue 100, cysteine, of the LDL-R protein encoded by the gene;

FIG. 16 shows another mutation of the LDL-R gene occurring at a site coding for amino acid residue 100, cysteine, of the LDL-R protein encoded by the gene;

FIG. 17 shows a deletion mutation occurring in nucleotides 382 and 383, of the LDL-R gene;

FIG. 18 shows an insertion mutation occurring at a position of nucleotide 390 of the LDL-R gene;

FIG. 19 shows a mutation of the LDL-R gene occurring at a site coding for amino acid residue 113, cysteine, of the LDL-R protein encoded by the gene;

FIG. 20 shows another mutation of the LDL-R gene occurring at a site coding for amino acid residue 113, cysteine, of the LDL-R protein encoded by the gene;

FIG. 21 shows a mutation of the LDL-R gene occurring at a site coding for amino acid residue 115, aspartic acid, of the LDL-R protein encoded by the gene;

FIG. 22 shows a deletion mutation occurring in nucleotides 578 to 584, of the LDL-R gene;

FIG. 23 shows an insertion mutation occurring at a position of nucleotide 682 of the LDL-R gene;

FIG. 24 shows a deletion mutation occurring in nucleotides 526 to 529, of the LDL-R gene;

FIG. 25 shows an insertion mutation occurring at a position of nucleotide 661 of the LDL-R gene;

FIG. 26 shows a mutation of the LDL-R gene occurring at a site coding for amino acid residue 207, glutamic acid, of the LDL-R protein encoded by the gene;

FIG. 27 shows another mutation of the LDL-R gene occurring at a site coding for amino acid residue 207, glutamic acid, of the LDL-R protein encoded by the gene;

FIG. 28 shows an insertion mutation occurring at a position of nucleotide 944 of the LDL-R gene;

FIG. 29 shows a deletion mutation occurring in nucleotide 948, cytosine, of the LDL-R gene;

FIG. 30 shows a mutation of the LDL-R gene occurring at a site coding for amino acid residue 317, cysteine, of the LDL-R protein encoded by the gene;

FIG. 31 shows another mutation of the LDL-R gene occurring at a site coding for amino acid residue 317, cysteine, of the LDL-R protein encoded by the gene;

FIG. 32 shows a deletion mutation occurring in nucleotides 1114 to 1134, of the LDL-R gene;

FIG. 33 shows an insertion mutation occurring at a position of nucleotide 1062 of the LDL-R gene;

FIG. 34 shows a mutation of the LDL-R gene occurring at a site coding for amino acid residue 336, glutamic acid, of the LDL-R protein encoded by the gene;

FIG. 35 shows a mutation of the LDL-R gene occurring at a site coding for amino acid residue 337, cysteine, of the LDL-R protein encoded by the gene;

FIG. 36 shows a mutation of the LDL-R gene occurring at a site coding for amino acid residue 356, cysteine, of the LDL-R protein encoded by the gene;

FIG. 37 shows a mutation of the LDL-R gene occurring at a site coding for amino acid residues 351 to 354 of the LDL-R protein encoded by the gene;

FIG. 38 shows a mutation of the LDL-R gene occurring at a site coding for-amino acid residue 358, cysteine, of the LDL-R protein encoded by the gene;

FIG. 39 shows a mutation occurring at nucleotide −10 (guanine) in the 5′-end-side acceptor region in intron 8 of the LDL-R gene;

FIG. 40 shows a mutation of the LDL-R gene occurring at a site coding for amino acid residue 382, phenylalanine, of the LDL-R protein encoded by the gene;

FIG. 41 shows a deletion mutation occurring in nucleotides 1202 to 1204, of the LDL-R gene;

FIG. 42 shows a mutation of the LDL-R gene occurring at a site coding for amino acid residue 385, arginine, of the LDL-R protein encoded by the gene;

FIG. 43 shows a mutation of the LDL-R gene occurring at a site coding for amino acid residue 387, glutamic acid, of the LDL-R protein encoded by the gene;

FIG. 44 shows an insertion mutation occurring at a position of nucleotide 1242 of the LDL-R gene;

FIG. 45 shows a mutation of the LDL-R gene occurring at a site coding for amino acid residue 401, leucine, of the LDL-R protein encoded by the gene;

FIG. 46 shows a mutation of the LDL-R gene occurring at a site coding for amino acid residue 410, alanine, of the LDL-R protein encoded by the gene;

FIG. 47 shows a mutation of the LDL-R gene occurring at a site coding for amino acid residue 412, aspartic acid, of the LDL-R protein encoded by the gene;

FIG. 48 shows a mutation of the LDL-R gene occurring at a site coding for amino acid residue 512, tryptophan, of the LDL-R protein encoded by the gene;

FIG. 49 shows a deletion mutation occurring in nucleotide 1599, of the LDL-R gene;

FIG. 50 shows an insertion mutation occurring at a position of nucleotide 1687 of the LDL-R gene;

FIG. 51 shows a deletion mutation occurring in nucleotides 1652 to 1662, of the LDL-R gene;

FIG. 52 shows a deletion mutation occurring in nucleotide 1655, thymine, of the LDL-R gene;

FIG. 53 shows a mutation of the LDL-R gene occurring at a site coding for amino acid residue 547, leucine, of the LDL-R protein encoded by the gene;

FIG. 54 shows a mutation from guanine to another base, occurring at nucleotide +1 (guanine) in a splice donor site of intron 11 starting from the nucleotide that is one base downstream the nucleotide 1705 of the LDL-R gene;

FIG. 55 shows another mutation from guanine to another base, occurring at nucleotide +1 (guanine) in a splice donor site of intron 11 starting from the nucleotide that is one base downstream the nucleotide 1705 of the LDL-R gene;

FIG. 56 shows a mutation of the LDL-R gene occurring at a site coding for amino acid residue 570, asparagine, of the LDL-R protein encoded by the gene;

FIG. 57 shows an insertion mutation occurring at a position of nucleotide 1779 of the LDL-R gene;

FIG. 58 shows a mutation of the LDL-R gene occurring at a site coding for amino acid residue 587, proline, of the LDL-R protein encoded by the gene;

FIG. 59 shows a mutation of the LDL-R gene occurring-at a site coding for amino acid residue 591, alanine, of the LDL-R protein encoded by the gene;

FIG. 60 shows a deletion mutation occurring in nucleotides 1870 to 1872, of the LDL-R gene;

FIG. 61 shows a mutation of the LDL-R gene occurring at a site coding for amino acid residue 612, arginine, of the LDL-R protein encoded by the gene;

FIG. 62 shows a deletion mutation occurring in nucleotide 1963, of the LDL-R gene;

FIG. 63 shows an insertion mutation occurring at a position of nucleotide 2035 of the LDL-R gene;

FIG. 64 shows a mutation of the LDL-R gene occurring at a site coding for amino acid residue 693, glutamic acid, of the LDL-R protein encoded by the gene;

FIG. 65 shows a deletion mutation occurring in nucleotides 2320 to 2340, of the LDL-R gene;

FIG. 66 shows a mutation of the LDL-R gene occurring at a site coding for amino acid residue 779, valine, of the LDL-R protein encoded by the gene;

FIG. 67 shows a mutation of the LDL-R gene occurring at a site coding for amino acid residue 790, lysine, of the LDL-R protein encoded by the gene;

FIG. 68 shows an insertion mutation occurring at a position of nucleotide 2412 of the LDL-R gene;

FIG. 69 shows a mutation of the LDL-R gene occurring at a site coding for amino acid residue 316, glutamic acid, of the LDL-R protein encoded by the gene;

FIG. 70 shows a genetic polymorphism of the LDL-R gene in which nucleotide 81, T, is substituted by C;

FIG. 71 shows a genetic polymorphism of the LDL-R gene in which nucleotide 636, C, is substituted by T;

FIG. 72 shows a genetic polymorphism of the LDL-R gene in which nucleotide 969, C, is substituted by T;

FIG. 73 shows a genetic polymorphism of the LDL-R gene in which nucleotide 1002, C, is substituted by T;

FIG. 74 shows a genetic polymorphism of the LDL-R gene in which nucleotide 1195, C, is substituted by T;

FIG. 75 shows a genetic polymorphism of the LDL-R gene in which nucleotide 1725, C, is substituted by T;

FIG. 76 shows a genetic polymorphism of the LDL-R gene in which nucleotide 1773, T, is substituted by C; and

FIG. 77 shows a genetic polymorphism of the LDL-R gene in which nucleotide 1959, C, is substituted by T.

### BEST MODE FOR CARRYING OUT THE INVENTION

Modes of the present invention will be described hereafter.

As mentioned above, the present invention is directed to a method of detecting arteriosclerosis and/or ischemic heart disease through detection of a risk factor for abnormality of lipid metabolism, and the underlying concept of the present invention resides in detection of abnormalities in the low-density lipoprotein receptor (hereinafter may be referred to as LDL-R) gene, which is an arteriosclerosis-associated gene.

Analyses of the LDL-R gene and the LDL-R protein encoded by the LDL-R gene have already been performed (Yamamoto T, et al., Cell 39, 27-38, 1984). The LDL-R nucleotide sequence and its corresponding amino acid sequence are shown in SEQ ID NO: 1.

Detailed analyses of relationships between mutations of the LDL-R gene and pathological phenomena—such as arteriosclerosis and ischemic heart diseases—that are considered to be attributable to abnormalities occurring in LDL-R will identify mutations of LDL-R gene useful in the practice of the detection method of the present invention. For example, gene mutations of interest can be identified if analysis of mutation sites or mutation frequency of the LDL-R gene or analysis of functions of the protein bearing the mutation is performed on combinations of “patients suffering arteriosclerosis or ischemic heart disease” and “healthy subjects.” Specific procedures of such analyses will be described in the “Examples” section hereinbelow.

In the context of the present invention, “gene mutation” means alteration occurring in a gene in the human chromosome, and more specifically means that the nucleotide sequence of the gene differs from that of the wild-type gene (the nucleotide sequence of a normal gene). When specific sites of a gene in its nucleotide sequence differ from one another in an individual-dependent manner, such events can generally be referred to as “genetic polymorphism.” According to the present invention, such genetic polymorphism also falls within the meaning of the “gene mutation.” The “gene mutation” can be identified through analyses, by employment of various approaches, of mutation frequency of the gene, expression level of mRNA, expression level of protein, functions of the protein, etc. Such gene mutation has been considered to occur at a frequency of about 1 in several hundreds of bases on average, and can be identified through direct or indirect analysis of a gene. Familial analysis regarding the gene mutation thus identified will determine whether a chromosome (an allele) is inherited from the paternal side or from the maternal side.

The alteration occurring in a mutation site of a gene is inherited from either the paternal side or the maternal side. When a base in the mutation site has undergone substitution to thereby manifest substitution of both alleles by different bases as compared with the case of the wild-type gene, such a case is referred to as a “homozygote,” whereas when the nucleotide sequence of one allele differs from that of the wild-type gene, such a case is referred to as a “heterozygote.”

As described above, the present inventors have so far identified the following genetic abnormalities which are found in the LDL-R gene and are correlated with risk factors concerning arteriosclerosis:

1. a mutation of an LDL-R gene coding for LDL-R protein, the mutation occurring at a site coding for amino acid residue 25, cysteine, of the LDL-R protein (for example, in the LDL-R gene, nucleotide 137, guanine, is substituted by cytosine, and the mentioned cysteine is changed to serine);

2. a deletion mutation occurring in nucleotides 156-160 of the LDL-R gene;

3. a mutation of the LDL-R gene occurring at a site coding for amino acid residue 50, glycine, of the LDL-R protein encoded by the gene (for example, in the LDL-R gene, nucleotide 211, guanine, is substituted by adenine, and the mentioned glycine is changed to arginine);

4. a mutation of the LDL-R gene occurring at a site coding for amino acid residue 74, cysteine, of the LDL-R protein encoded by the gene (for example, (1) in the LDL-R gene, nucleotide 283, thymine, is substituted by adenine, and the mentioned cysteine is changed to serine, and (2) in the LDL-R gene, nucleotide 285, cytosine, is substituted by adenine, with the site coding for cysteine having been changed into a stop codon);

5. a deletion mutation occurring in nucleotide 314, cytosine, of the LDL-R gene;

6. a mutation of the LDL-R gene occurring at a site coding for amino acid residue 81, glutamine, of the LDL-R protein encoded by the gene (for example, in the LDL-R gene, nucleotide 304, cytosine, is substituted by thymine, with the site coding for glutamine having been changed into a stop codon);

7. a mutation of the LDL-R gene occurring at a site coding for amino acid residue 88, cysteine, of the LDL-R protein encoded by the gene (for example, in the LDL-R gene, nucleotide 326, guanine, is substituted by cytosine, and the mentioned cysteine is changed to serine);

8. a mutation of the LDL-R gene occurring at a site coding for amino acid residue 90, glutamine, of the LDL-R protein encoded by the gene (for example, in the LDL-R gene, nucleotide 331, cytosine, is substituted by thymine, with the site coding for glutamine having been changed into a stop codon);

9. a mutation of the LDL-R gene occurring at a site coding for amino acid residue 94, arginine, of the LDL-R protein encoded by the gene (for example, in the LDL-R gene, nucleotide 344, guanine, is substituted by adenine, and the mentioned arginine is changed to histidine);

10. a deletion mutation occurring in nucleotides 355-361, of the LDL-R gene;

11. a mutation of the LDL-R gene occurring at a site coding for amino acid residue 100, cysteine, of the LDL-R protein encoded by the gene (for example, (1) in the LDL-R gene, nucleotide 361, thymine, is substituted by guanine, and the mentioned cysteine is changed to glycine, and (2) in the LDL-R gene, nucleotide 363, cytosine, is substituted by adenine, with the site coding for cysteine having been changed into a stop codon);

12. a deletion mutation occurring in nucleotides 382-383, of the LDL-R gene;

13. an insertion mutation occurring at a position of nucleotide 390 of the LDL-R gene;

14. a mutation of the LDL-R gene occurring at a site coding for amino acid residue 113, cysteine, of the LDL-R protein encoded by the gene (for example, (1) in the LDL-R gene, nucleotide 401, guanine, is substituted by thymine, and the mentioned cysteine is changed to phenylalanine, and (2) in the LDL-R gene, nucleotide 400, thymine, is substituted by cytosine, and the mentioned cysteine is changed to arginine);

15. a mutation of the LDL-R gene occurring at a site coding for amino acid residue 115, aspartic acid, of the LDL-R protein encoded by the gene (for example, in the LDL-R gene, nucleotide 406, guanine, is substituted by adenine, and the mentioned aspartic acid is changed to asparagine);

16. a mutation of the LDL-R gene occurring at a site coding for amino acid residue 119, glutamic acid, of the LDL-R protein encoded by the gene (for example, in the LDL-R gene, nucleotide 418, guanine, is substituted by adenine, and the mentioned glutamic acid is changed to lysine);

17. a deletion mutation occurring in nucleotides 578-584, of the LDL-R gene;

18. an insertion mutation occurring at a position of nucleotide 682 of the LDL-R gene;

19. a deletion mutation occurring in nucleotides 526-529, of the LDL-R gene;

20. an insertion mutation occurring at a position of nucleotide 661 of the LDL-R gene;

21. a mutation of the LDL-R gene occurring at a site coding for amino acid residue 207, glutamic acid, of the LDL-R protein encoded by the gene (for example, (1) in the LDL-R gene, nucleotide 862, guanine, is substituted by adenine, and the mentioned glutamic acid is changed to lysine, and (2) in the LDL-R gene, nucleotide 682, guanine, is substituted by cytosine, and the mentioned glutamic acid is changed to glutamine);

22. an insertion mutation occurring at a position of nucleotide 944 of the LDL-R gene;

23. a deletion mutation occurring in nucleotide 948, cytosine, of the LDL-R gene;

24. a mutation of the LDL-R gene occurring at a site coding for amino acid residue 317, cysteine, of the LDL-R protein encoded by the gene (for example, (1) in the LDL-R gene, nucleotide 1012, thymine, is substituted by adenine, and the mentioned cysteine is changed to serine, and (2) in the LDL-R gene, nucleotide 1012, thymine, is substituted by cytosine, and the mentioned cysteine is changed to arginine);

25. a deletion mutation occurring in nucleotides 1114-1134, of the LDL-R gene;

26. an insertion mutation occurring at a position of nucleotide 1062 of the LDL-R gene;

27. a mutation of the LDL-R gene occurring at a site coding for amino acid residue 336, glutamic acid, of the LDL-R protein encoded by the gene (in the LDL-R gene, nucleotide 1069, guanine, is substituted by thymine, with the site coding for glutamic acid having been changed into a stop codon);

28. a mutation of the LDL-R gene occurring at a site coding for amino acid residue 337, cysteine, of the LDL-R protein encoded by the gene (for example, in the LDL-R gene, nucleotide 1072, thymine, is substituted by cytosine, and the mentioned cysteine is changed to arginine);

29. a mutation of the LDL-R gene occurring at a site coding for amino acid residue 356, cysteine, of the LDL-R protein encoded by the gene (for example, in the LDL-R gene, nucleotide 1130, guanine, is substituted by adenine, and the mentioned cysteine is changed to tyrosine);

30. a mutation of the LDL-R gene occurring at a site coding for amino acid residues 351-354, glutamic acid—glycine—glycine—tyrosine, of the LDL-R protein encoded by the gene;

31. a mutation of the LDL-R gene occurring at a site coding for amino acid residue 358, cysteine, of the LDL-R protein encoded by the gene (for example, in the LDL-R gene, nucleotide 1136, guanine, is substituted by adenine, and the mentioned cysteine is changed to tyrosine);

32. a mutation occurring at nucleotide −10 (guanine) in the 5′-end-side acceptor region in intron 8 of the LDL-R gene;

33. a mutation of the LDL-R gene occurring at a site coding for amino acid residue 382, phenylalanine, of the LDL-R protein encoded by the gene (for example, in the LDL-R gene, nucleotide 1207, thymine, is substituted by cytosine, and the mentioned phenylalanine is changed to leucine);

34. a mutation occurring in nucleotide 1599, of the LDL-R gene;

35. a deletion mutation occurring in nucleotides 1202-1204, of the LDL-R gene;

36. a mutation of the LDL-R gene occurring at a site coding for amino acid residue 385, arginine, of the LDL-R protein encoded by the gene (for example, in the LDL-R gene, nucleotide 1216, cytosine, is substituted by thymine, and the mentioned arginine is changed to tryptophan);

37. a mutation of the LDL-R gene occurring at a site coding for amino acid residue 387, glutamic acid, of the LDL-R protein encoded by the gene (for example, in the LDL-R gene, nucleotide 1222, guanine, is substituted by adenine, and the mentioned glutamic acid is changed to lysine);

38. an insertion mutation occurring at a position of nucleotide 1242 of the LDL-R gene;

39. a mutation of the LDL-R gene occurring at a site coding for amino acid residue 401, leucine, of the LDL-R protein encoded by the gene (for example, in the LDL-R gene, nucleotide 1265, thymine, is substituted by guanine, and the mentioned leucine is changed to arginine);

40. a mutation of the LDL-R gene occurring at a site coding for amino acid residue 410, alanine, of the LDL-R protein encoded by the gene (for example, in the LDL-R gene, nucleotide 1291, guanine, is substituted by adenine, and the mentioned alanine is changed to threonine);

41. a mutation of the LDL-R gene occurring at a site coding for amino acid residue 412, aspartic acid, of the LDL-R protein encoded by the gene (for example, in the LDL-R gene, nucleotide 1297, guanine, is substituted by cytosine, and the mentioned aspartic acid is changed to histidine);

42. a mutation of the LDL-R gene occurring at a site coding for amino acid residue 512, tryptophan, of the LDL-R protein encoded by the gene (for example, in the LDL-R gene, nucleotide 1599, guanine, is substituted by adenine, and the mentioned site coding for tryptophan is changed to a stop codon);

43. a deletion mutation occurring in nucleotides 1652-1662, of the LDL-R gene;

44. a deletion mutation occurring in nucleotide 1655, thymine, of the LDL-R gene;

45. an insertion mutation occurring at a position of nucleotide 1687 of the LDL-R gene;

46. a mutation of the LDL-R gene occurring at a site coding for amino acid residue 547, leucine, of the LDL-R protein encoded by the gene (for example, in the LDL-R gene, nucleotide 1702, cytosine, is substituted by guanine, and the mentioned leucine is changed to valine);

47. a mutation occurring at nucleotide +1 (guanine) in a splice donor site of intron 11 starting from the nucleotide that is next to nucleotide 1705 of the LDL-R gene;

48. a mutation occurring at nucleotide +2 (thymine) in a splice donor site of intron 12 starting from the nucleotide that is next to nucleotide 1845 of the LDL-R gene;

49. a mutation of the LDL-R gene occurring at a site coding for amino acid residue 556, tryptophan, of the LDL-R protein encoded by the gene (for example, in the LDL-R gene, nucleotide 1731, guanine, is substituted by thymine, and the mentioned tryptophan is changed to cysteine);

50. a mutation of the LDL-R gene occurring at a site coding for amino acid residue 570, asparagine, of the LDL-R protein encoded by the gene (for example, in the LDL-R gene, nucleotide 1772, adenine, is substituted by guanine, and the mentioned asparagine is changed to serine);

51. an insertion mutation occurring at a position of nucleotide 1779 of the LDL-R gene;

52. a mutation of the LDL-R gene occurring at a site coding for amino acid residue 587, proline, of the LDL-R protein encoded by the gene (for example, in the LDL-R gene, nucleotide 1822, cytosine, is substituted by thymine, and the mentioned proline is changed to serine);

53. a mutation of the LDL-R gene occurring at a site coding for amino acid residue 591, alanine, of the LDL-R protein encoded by the gene (for example, in the LDL-R gene, nucleotide 1834, guanine, is substituted by thymine, and the mentioned alanine is changed to serine);

54. a deletion mutation occurring in nucleotides 1870-1872, of the LDL-R gene;

55. a mutation of the LDL-R gene occurring at a site coding for amino acid residue 612, arginine, of the LDL-R protein encoded by the gene (for example, in the LDL-R gene, nucleotide 1897, cytosine, is substituted by thymine, and the mentioned arginine is changed to cysteine);

56. a deletion mutation occurring in nucleotide 1963, of the LDL-R gene;

57. an insertion mutation occurring at a position of nucleotide 2035 of the LDL-R gene;

58. a mutation of the LDL-R gene occurring at a site coding for amino acid residue 664, proline, of the LDL-R protein encoded by the gene (for example, in the LDL-R gene, nucleotide 2054, cytosine, is substituted by thymine, and the mentioned proline acid is changed to leucine);

59. a mutation of the LDL-R gene occurring at a site coding for amino acid residue 693, glutamic acid, of the LDL-R protein encoded by the gene (for example, in the LDL-R gene, nucleotide 2140, guanine, is substituted by adenine, and the mentioned glutamic acid is changed to lysine);

60. a deletion mutation occurring in nucleotides 2320-2340, of the LDL-R gene;

61. a mutation of the LDL-R gene occurring at a site coding for amino acid residue 779, valine, of the LDL-R protein encoded by the gene (for example, in the LDL-R gene, nucleotide 2398, guanine, is substituted by adenine, and the mentioned valine is changed to methionine);

62. a mutation of the LDL-R gene occurring at a site coding for amino acid residue 790, lysine, of the LDL-R protein encoded by the gene (for example, in the LDL-R gene, nucleotide 2431, adenine, is substituted by thymine, and the mentioned site coding for lysine is changed to a stop codon);

63. an insertion mutation occurring at a position of nucleotide 2412 of the LDL-R gene;

64. a mutation of the LDL-R gene occurring at a site coding for amino acid residue 829, alanine, of the LDL-R protein encoded by the gene (for example, in the LDL-R gene, nucleotide 2579, cytosine, is substituted by thymine, and the mentioned alanine is changed to valine); and

65. a mutation of the LDL-R gene occurring at a site coding for amino acid residue 316, glutamic acid, of the LDL-R protein encoded by the gene (for example, in the LDL-R gene, nucleotide 1010, thymine, is substituted by cytosine, and the mentioned glutamic acid is changed to glycine).

Alterations occurring in abnormal sites of a gene can be detected through any suitable conventional method, such as RFLP employing Southern blotting; PCR-RFLP; HET (hetero duplex analysis); DGGE (denaturing gradient gel electrophoresis); DS (direct sequencing); CCM (chemical cleavage mismatching); CDI (carbodiimide modification); an analysis technique for single-stranded conformation polymorphism making use of PCR (PCR-SSCP; hereinafter may be referred to simply as the SSCP method), or PCR/GC-clamping (see, for example, “Biomanual Series 1, Basic Techniques of Genetic Engineering,” edited by Miyabi Yamamoto, published by Yodo-sha (1993), and particularly regarding the PCR/GC-clamping, see, for example, “Genomic Analysis: A Practical Approach,” Myers, R. M., Shefield, V., and Cox, D. R. (1988), K. Davies, ed. IRL Press Limited, Oxford, pp. 95-139). Of these, PCR/GC-clamping is preferred, in that it enables convenient and accurate identification of a genetic abnormality.

Specifically, PCR/GC-clamping is a modified version of DGGE (note: DGGE is a method of detecting base substitution of DNA on polyacrylamide gel containing a DNA denaturant at linear gradient concentrations, wherein the method makes use of the mobility difference stemming from difference between the concentration of a DNA denaturant required for denaturing DNA of a double-stranded DNA fragment containing a base substitution and the concentration of the DNA denaturant required for denaturing DNA of a double-stranded DNA fragment containing no base substitution), and according to the modified version, the drawback involved in DGGE experienced in the case of a plurality of base substitutions; i.e., disabled detection of the base substitution of the domain lastly fused on the polyacrylamide gel, is overcome by ligating a region of high GC content (GC-clamp) with a DNA fragment carrying the base substitutions to be detected (see, for example, Shefield, V. C. et al. (1989) Proc. Natl. Acad. Sci. USA 86: 232-236).

Therefore, although the basic procedure of the PCR/GC-clamping technique is analogous to DGGE, there must be performed an additional step for adding a GC-clamp to the DNA fragment for which base substitution is to be detected.

According to the present invention, no particular limitations are imposed on the source of DNA molecules for which alteration in a genetically abnormal site of the LDL-R is to be detected, so long as it constitutes somatic cells of a test subject (patient). Blood samples such as peripheral blood samples or leukocyte samples are preferably used in the present invention.

From sample cells collected from a test subject, genomic DNA is extracted by a known method, and with regard to the thus-obtained genomic DNA, alteration occurring in a certain locus of the gene (specifically, substitution of a base at a specific abnormality-carrying locus of the gene) is detected.

When the above detection procedure reveals the presence of alteration in the mentioned specific locus of the gene, the alteration is correlated with lipid metabolic disorder or further with arteriosclerosis and/or ischemic heart disease, to thereby detect a risk factor for arteriosclerosis and/or ischemic heart disease. The concept of “detection of a risk factor for arteriosclerosis and/or ischemic heart disease” encompasses not only detection of the arteriosclerosis and/or ischemic heart disease currently suffered by the patient, but also high possibility of future onset of arteriosclerosis and/or ischemic heart disease. In other words, even in the case where arteriosclerosis or ischemic heart disease is not currently identified, if alteration is observed in a specific locus of the LDL-R gene, LDL-R cannot fully play its intrinsic roles in, for example, metabolism of serum lipids, thereby increasing the possibility of onset of arteriosclerosis and/or ischemic heart disease. Thus, according to the present detection method, such high possibility indicative of future arteriosclerosis and/or ischemic heart disease can also be detected as a risk factor for arteriosclerosis and/or ischemic heart disease.

When LDL-R does not function properly, serum lipids often show elevated LDL-C levels. Generally speaking, LDL-C is acknowledged to be “bad cholesterol,” and therefore, clinical institutions caution subjects when LDL-C level in serum lipids is found high. Early proper treatment is important particularly for FH patients, but, as described above, heterozygous FH patients rarely show prominently high LDL-C levels during childhood. Therefore, partially as a result of popularization of the westernized dietary style in recent years, discernment between FH from transient hyperlipemia attributed to changes in dietary style of nonhereditary nature is difficult. When the present detection method is used to test a subject who is suspected whether he has FH because of his relatively high LDL-C level in serum lipids, risk factors concerning oneset of hidden arteriosclerosis and/or ischemic heart disease can be properly detected.

When the present detection method reveals one or more of the above-recited specific alterations in the LDL-R gene from a patient, and in addition, the patient exhibits pathological conditions closely related to arteriosclerosis and/or ischemic heart disease, such as diabetes or hypertension, lethal events caused by future arteriosclerosis can be prevented by starting a combination treatment of conventional therapy for such pathological conditions and therapy for arteriosclerosis. Moreover, when the present detection method reveals one or more of the above-recited specific alterations in the LDL-R gene from a patient who is seemingly a healthy subject, provision of measures for preventing the onset of arteriosclerosis or ischemic heart disease, such as providing suggestions for, or administration of, improved diet and proper exercise, may reduce risk factors for the onset of arteriosclerosis and/or ischemic heart disease of the patient.

In practice of the present detection method, when specific abnormality or abnormalities occurring in one or more specific loci of the LDL-R gene as newly identified by the present invention are detected in combination with conventionally identified abnormality or abnormalities occurring in one or more specific loci of the LDL-R gene, it is possible to attain more accurate detection of risk factors for the onset of arteriosclerosis and/or ischemic heart disease.

### EXAMPLES

The present invention will next be described in more detail by way of examples.

### Method for Analysis of the LDL-R Gene

### Preparation of Monocytes

An ACD-added peripheral blood sample (10 mL) collected from a test subject is placed to form a layer in a SEPARATE L (5 mL, product of Wako Pure Chemical Industries, Ltd.), and then subjected to specific gravity centrifugation at 1,200 rpm for 60 minutes, to thereby separate monocytes. By use of an RPMI medium (10 mL, product of Lifetech Oriental, Inc.), the obtained monocytes are centrifuged twice at 1,500 rpm for 10 minutes, and washed. Subsequently, an RPMI medium supplemented with 1%-fatty-acid-depleted bovine serum albumin (BSA) is added, to thereby prepare a solution containing 5×10⁶ monocytes/mL. The thus-prepared monocytes are incubated in an incubator (5% CO₂) at 37° C. for three days. Thereafter, the monocytes are washed twice with PBS, and suspended in PBS containing 1-mM CaCl₂ (0.5 mL). An aliquot (0.1 mL) of the monocyte suspension is placed in a Microfuge tube (product of Eppendorf), to which a Dil-LDL (10 μg/mL, product of Molecular Probes) is added for reaction at 37° C. for two hours. After completion of reaction, the cells are washed twice with PBS, and finally suspended in PBS supplemented with 1% BSA (0.1 mL). A diluted anti-LDL-R antibody (IgG-C7) solution (10 μL) is added to another aliquot (0.1 mL) of the monocyte suspension for reaction at 4° C. for 30 minutes. After completion of reaction, three washings are performed with ice-cold PBS containing 1% BSA, to thereby remove unreacted antibodies. After the washings, PE-labeled anti-mouse IgG antibodies are added for reaction at 4° C. for 30 minutes. After completion of reaction, three washings are performed with ice-cold PBS containing 1% BSA, to thereby remove unreacted antibodies and obtain 500 μL of a cell suspension in PBS supplemented with 1% BSA.

### Measurement by Means of a Flow Cytometer

Dil-LDL taken in cells and antibodies which have been bound to LDL-R expressed on the cell membrane are detected through measuring fluorescence intensity of the cells by means of a flow cytometer (FACScan, product of Becton Dickinson). Briefly, the cells which have been reacted as described above are subjected to flow cytometry under the following conditions: laser power=15 W, excitation wavelength=488 nm, wavelength=530 nm, PMT voltage=500 mV. In the measurement, gating is set for the lymphocyte region based on forward scatter (FSC) and side scatter (SSC) parameters for measurement of the intensity of FL1. The fluorescence intensity of the cells is obtained by measuring the fluorescence intensity of ten thousands cells. The thus-obtained fluorescence intensity of the cells is analyzed by use of analysis software CELLQUEST installed in the computer connected to the flow cytometer.

### Quantitation of the Protein Amount and Activity of LDL-R

As described above, fluorescence intensities of the cells obtained from an individual are measured by flow cytometry, and from the results of the measurement, average fluorescence intensities are calculated. The average fluorescence intensities are considered to represent the protein amount and the activity of the LDL-R expressed in the cells. Specifically, an average fluorescence intensity of the cells obtained by use of antibodies is considered to represent the protein amount of the LDL-R expressed in the cells, and the fluorescence intensity of the cells incorporating the fluorescence dye Dil-LDL is considered to represent the LDL-R activity. Both the protein amount and the activity of LDL-R are expressed in terms of percent (%) with respect to the average fluorescence intensity of cells obtained from 2 to 4 healthy subjects whose serum lipid levels are normal. That is, the protein amount and the activity of LDL-R of an individual are calculated by the following equations.

“Amount of LDL-R protein of an individual” (%)={“fluorescence intensity as measured for the individual obtained by use of antibodies”/“average fluorescence intensity as measured for healthy subjects by use of antibodies”}×100

“Activity of LDL-R of an individual” (%)=(“fluorescence intensity as measured for the individual obtained by use of Dil-LDL”/“average fluorescence intensity as measured for healthy subjects by use of Dil-LDL”}×100<PCR>

Genomic DNA of an individual is extracted from peripheral leukocytes by use of a DNA extraction kit (QIAamp DNA Blood kit, product of Qiagen). The nucleic acids in the promoter region and exon regions of exon 1 to exon 18 of LDL-R gene are amplified by use of the following oligonucleotides (see also SEQ ID NOs: 2-43):

| | |
|---|---|
| promoter region: | |
| GAGTGGGAATCAGAGCTTCACGGGT | (SEQ ID NO: 2) |
| CCACGTCATTTACAGCATTTCAATG | (SEQ ID NO: 3) |
| exon 1: | |
| ACTCCTCCCCCTGCTAGAAACCTCA | (SEQ ID NO: 4) |
| TTCTGGCGCTTGGAGCAAGCCTTAC | (SEQ ID NO: 5) |
| exon 2: | |
| CCTTTCTCCTTTTCCTCTCTCTCAG | (SEQ ID NO: 6) |
| AAAATAAATGCATATCATGCCCAAA | (SEQ ID NO: 7) |
| exon 3: | |
| TGACAGTTCAATCCTGTCTCTTCTG | (SEQ ID NO: 8) |
| ATAGCAAAGGCAGGGCCACACTTAC | (SEQ ID NO: 9) |
| exon 4A: | |
| GTTGGGAGACTTCACACGGTGATGG | (SEQ ID NO: 10) |
| ACTTAGGCAGTGGAACTCGAAGGCC | (SEQ ID NO: 11) |
| exon 4B: | |
| CCCCAGCTGTGGGCCTGCGACAACG | (SEQ ID NO: 12) |
| GGGGGAGCCCAGGGACAGCTGATAG | (SEQ ID NO: 13) |
| exon 5: | |
| CAACACACTCTGTCCTGTTTTCCAG | (SEQ ID NO: 14) |
| GGAAAACCAGATGGCCAGCGCTCAC | (SEQ ID NO: 15) |
| exon 6: | |
| TCCTTCCTCTCTCTGGCTCTCACAG | (SEQ ID NO: 16) |
| GCAAGCCGCCTGCACCGAGACTCAC | (SEQ ID NO: 17) |
| exon 7: | |
| AGTCTGACTCCCTGGCCCTGCGCAG | (SEQ ID NO: 18) |
| AGGGCTCAGTCCACCGGGGAATCAC | (SEQ ID NO: 19) |
| exon 8: | |
| CCAAGCCTCTTTCTCTCTCTTCCAG | (SEQ ID NO: 20) |
| CCACCCGCCGCCTTCCCGTGCTCAC | (SEQ ID NO: 21) |
| exon 9: | |
| TCCATCGACGGGTCCCCTCTGACCC | (SEQ ID NO: 22) |
| AGCCCTCATCTCACCTGCGGGCCAA | (SEQ ID NO: 23) |
| exon 10A: | |
| AGATGAGGGCTCCTGGTGCGATGCC | (SEQ ID NO: 24) |
| GCCCTTGGTATCCGCAACAGAGACA | (SEQ ID NO: 25) |
| exon 10B: | |
| GATCCACAGCAACATCTACTGGACC | (SEQ ID NO: 26) |
| AGCCCTCAGCGTCGTGGATACGCAC | (SEQ ID NO: 27) |
| exon 11: | |
| CAGCTATTCTCTGCTCTCCCACCAG | (SEQ ID NO: 28) |
| TGGGACGGCTGTCCTCGCAACATAC | (SEQ ID NO: 29) |
| exon 12: | |
| GCACGTGACCTCTCCTTATCCACTT | (SEQ ID NO: 30) |
| CACCTAAGTGCTTCGATCTCGTACG | (SEQ ID NO: 31) |
| exon 13: | |
| GTCATCTTCCTTGCTCCCTGTTTAG | (SEQ ID NO: 32) |
| GTTTCCACAAGGAGGTTTCAAGGTT | (SEQ ID NO: 33) |
| exon 14: | |
| CCTGACTCCGCTTCTTCTGCCCCAG | (SEQ ID NO: 34) |
| CGCAGAAACAAGGCGTGTGCCACAC | (SEQ ID NO: 35) |
| exon 15: | |
| GAAGGGCCTGCAGGCACGTGGCACT | (SEQ ID NO: 36) |
| GTGTGGTGGCGGGCCCAGTCTTTAC | (SEQ ID NO: 37) |
| exon 16: | |
| CCTCACTCTTGCTTCTCTCCTGCAG | (SEQ ID NO: 38) |
| CGCTGGGGGACCGGCCCGCGCTTAC | (SEQ ID NO: 39) |
| exon 17: | |
| TGACAGAGCGTGCCTCTCCCTACAG | (SEQ ID NO: 40) |
| GCTTTCTAGAGAGGGTCACACTCAC | (SEQ ID NO: 41) |
| exon 18: | |
| TCCGCTGTTTACCATTTGTTGGCAG | (SEQ ID NO: 42) |
| AATAAAACAAGGCCGGCGAGGTCTC | (SEQ ID NO: 43) |

Mutations of the LDL-R gene can be analyzed through denaturing gradient gel electrophoresis (DGGE) on polyacrylamide gel. Briefly, PCR is performed by use of oligonucleotides prepared by adding a GC clamp of 40 bp to antisense primers in the following steps: mixing 0.5 μL DNA (0.5 g DNA) with 49 μL PCR reaction mixture (10 mM Tris-HCl (pH 8.4), 50 mM KCl, 0.2 mM dNTP, each of the primers (50 pmol)) and 0.5 μL Taq DNA polymerase (2.5 unit, product of Roche); denaturing the mixture (95° C., 5 minutes); performing 25 cycles of treatment, each cycle consisting of denaturation (95° C., 30 seconds), annealing (65° C., 30 seconds), and elongation (72° C., 90 seconds); and elongating (72° C., 10 minutes). Then, the PCR product is electrophoresed by use of 3% agarose gel, and the resultant gel is stained with ethidium bromide. The electrophoresis bands are investigated by means of a UV trans-illuminator.

### Mutation Analysis of the Gene Through Denaturing Gradient Gel Electrophoresis (DGGE) on Polyacrylamide Gel

Mutations of the LDL-R gene are analyzed through a modified version of the denaturing gradient polyacrylamide gel electrophoresis described by Top, et al. (Top B., et al., Hum Genet, 91; 480-484, 1993). Briefly, each of the exons is amplified by use of the corresponding primer which has been added to a GC clamp, and the obtained PCR product is electrophoresed at 150 V for 16 hours by use of 9% polyacrylamide gel with a denaturing gradient (40 to 80%). After completion of electrophoresis, the gel is stained with ethidium bromide, and the obtained bands are investigated by means of a UV trans-illuminator.

As a result of the above-described DGGE, a PCR product carrying a mutation is detected as an abnormal band pattern. Specifically, in the case of a heterozygote in which one allele carries a mutation, a heteroduplex band and a homoduplex band are detected, whereas in the case of a homozygote in which both alleles carry mutations, a homoduplex band is detected at a locus different from that determined for the gene of a wild type.

### Determination of Nucleotide Sequence

Each of the exons showing abnormal band patterns (i.e., a homoduplex band at a locus different from that determined for the gene of a wild type, or a heteroduplex band) as a result of the above-described DGGE is subjected to PCR direct sequencing, to thereby determine its nucleotide sequence. Briefly, the exon is again amplified through PCR, and the PCR product is electrophoresed on 3% agarose gel, and cut out from the gel. Subsequently, the thus-obtained PCR product of interest is purified by use of a QIAamp (product of Qiagen), and the purified PCR product is labeled with a fluorescent marker by use of a Big Dye Terminator Cycle Sequence Kit (product of Applied Biosystems), and nucleotide sequencing is performed by use of an ABI 377 DNA sequencer (product of PE Biosystems)

### Clinical Analysis

### The Amount of Serum Lipid and LDL-R Protein and LDL-R Activity of Patients Suffering FH

LDL-R of 73 patients (31 male, 42 female) who had been clinically diagnosed as suffering familial hyperlipemia (FH) was analyzed. The patients' data are as follows: age=41.4±14.7 years old (mean±SD), serum cholesterol 280.0±65.9 mg/dL, neutral lipid=102.7±56.0 mg/dL, HDL cholesterol=46.6±18.5 mg/dL, amount of LDL-R protein=54.7±20.9%, LDL-R activity=59.1±14.1%.

### Mutation Analysis of LDL-R Gene Through DGGE

### Analysis of the LDL-R Gene of Patients Suffering FH

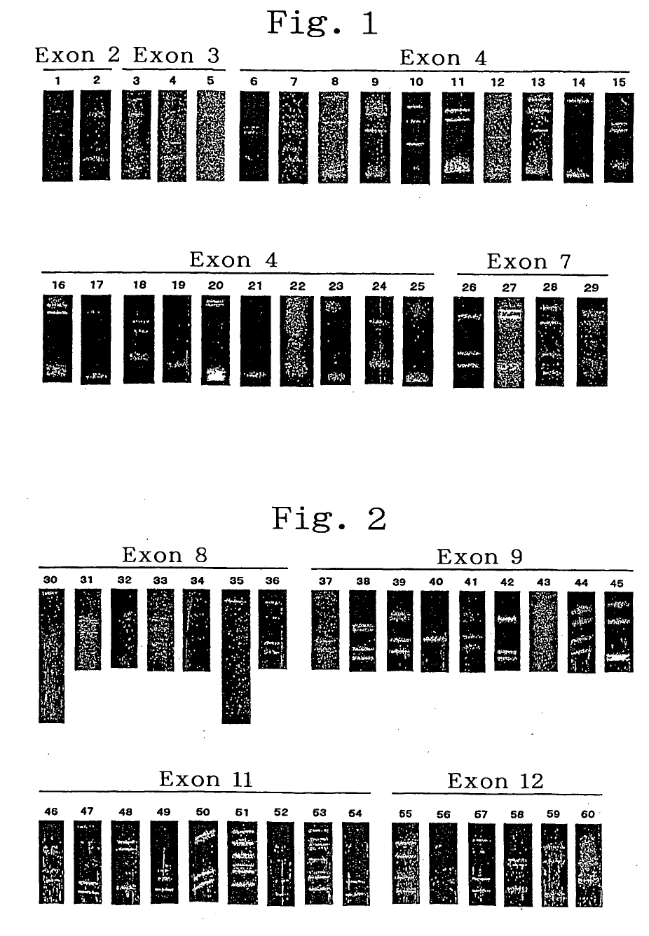

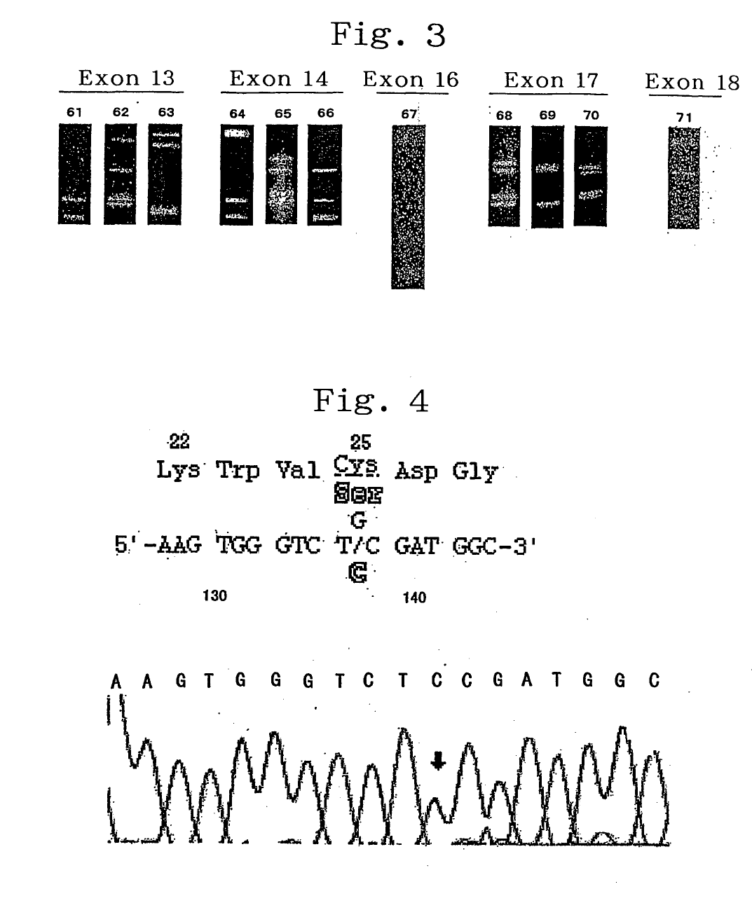

In the above-described mutation analysis through DGGE, the following exons were found to exhibit abnormal band patterns: 2 abnormal band patterns for exon 2, 3 for exon 3, 20 for exon 4, and 4 for exon 7 (details of these are shown in the electrophoresis patterns in FIG. 1); 7 for exon 8, 9 for exon 9, and 6 for exon 12 (details of these are shown in the electrophoresis patterns in FIG. 2); and 3 for exon 13, 3 for exon 14, 1 for exon 16, 3 for exon 17, and 1 for exon 18 (details of these are shown in the electrophoresis patterns in FIG. 3). Nucleotide sequences of the exons of the LDL-R genes of the patients suffering FH were determined through sequencing by use of dideoxynucleotide labeled with [³⁵S] dATP or sequencing by use of deoxynucleotide labeled with a fluorescent marker.

As a result, FH case 1 was found to have a substitution G→C at nucleotide 137 (G) in exon 2 of the LDL-R gene, which resulted in a mutation of the LDL-R protein such that the amino acid residue Cys encoded by the 25th codon was replaced by Ser (FIG. 4).

These results have clarified that a risk factor indicating abnormality in lipid metabolism can be detected through correlation between the gene mutation occurring at a site coding for amino acid residue 25, cysteine, of the LDL-R protein and abnormality in lipid metabolism.

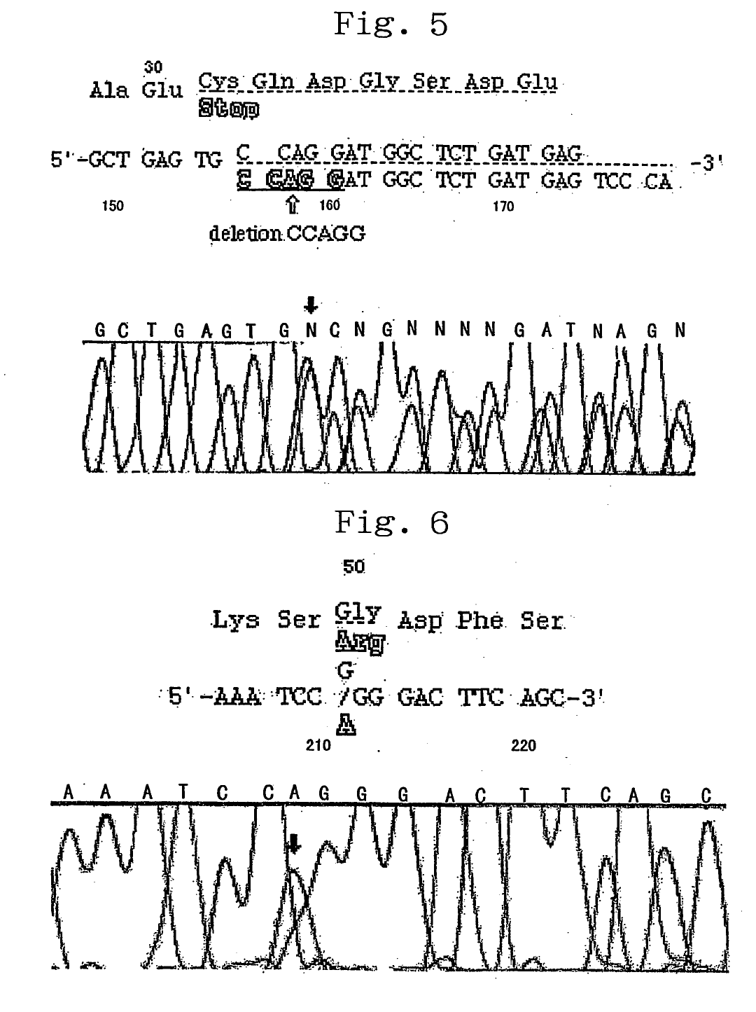

FH case 2 was found to have a loss of 5 bases corresponding to nucleotides 156 to 160 (CCAGG) in exon 2 of the LDL-R gene, producing a stop codon at codon 31 of the site of deletion (FIG. 5). This deletion mutation anticipates production of an abnormal LDL-R protein consisting of 30 amino acid residues.

Thus, the results have clarified that a risk factor indicating abnormality in lipid metabolism can be detected through correlation between the deletion mutation occurring in nucleotides 156 to 160 of the LDL-R gene and abnormality in lipid metabolism.

FH case 3,was found to have a substitution G→A at nucleotide 211 (G) in exon 3 of the LDL-R gene, which resulted in a mutation of the LDL-R protein such that the amino acid residue Gly encoded by the 50th codon was replaced by Arg (FIG. 6).

These results have clarified that a risk factor indicating abnormality in lipid metabolism can de detected through correlation between the gene mutation occurring at a site coding for amino acid residue 50, glycine, of the LDL-R protein and abnormality in lipid metabolism.

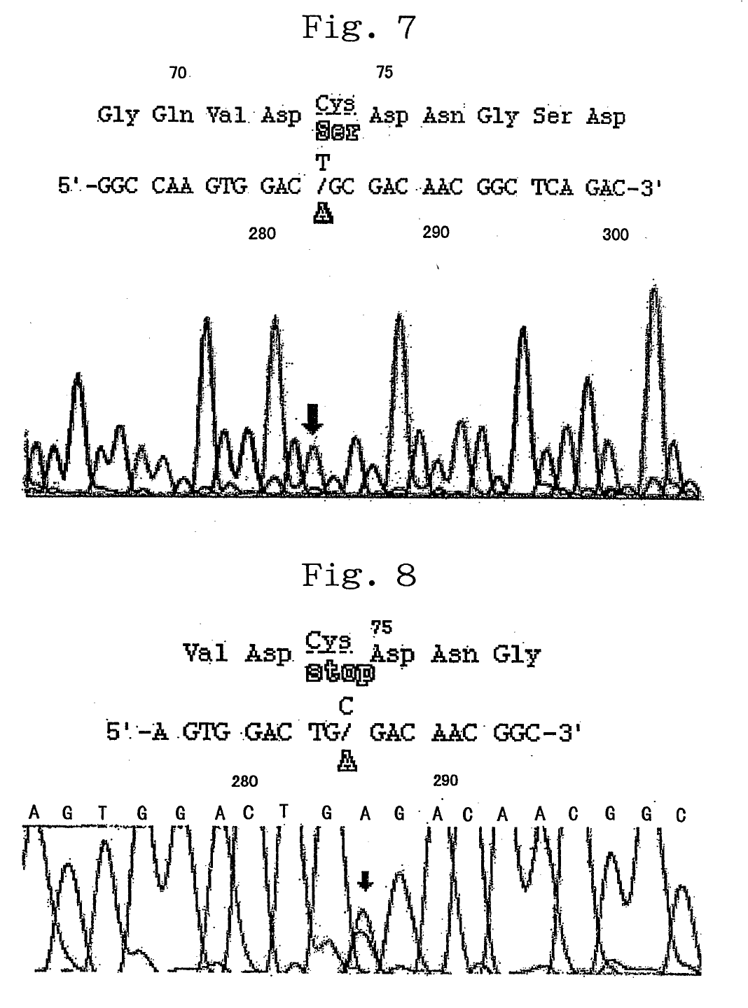

FH case 4 was found to have a substitution T→A at nucleotide 283 (T) in exon 3 of the LDL-R gene, which resulted in a mutation of the LDL-R protein such that the amino acid residue Cys encoded by the 74th codon was replaced by Ser (FIG. 7).

FH case 5 was found to have a substitution C→A at nucleotide 285 (C) in exon 3 of the LDL-R gene, which resulted in a mutation of the LDL-R protein wherein the 74th codon coding for the amino acid residue Cys was changed to a stop codon (FIG. 8). This base substitution anticipates production of an abnormal LDL-R protein consisting of 73 amino acid residues.

The results from FH cases 4 and 5 have clarified that a risk factor indicating abnormality in lipid metabolism can de detected through correlation between the gene mutation occurring at a site coding for amino acid residue 74, cysteine, of the LDL-R protein and abnormality in lipid metabolism.

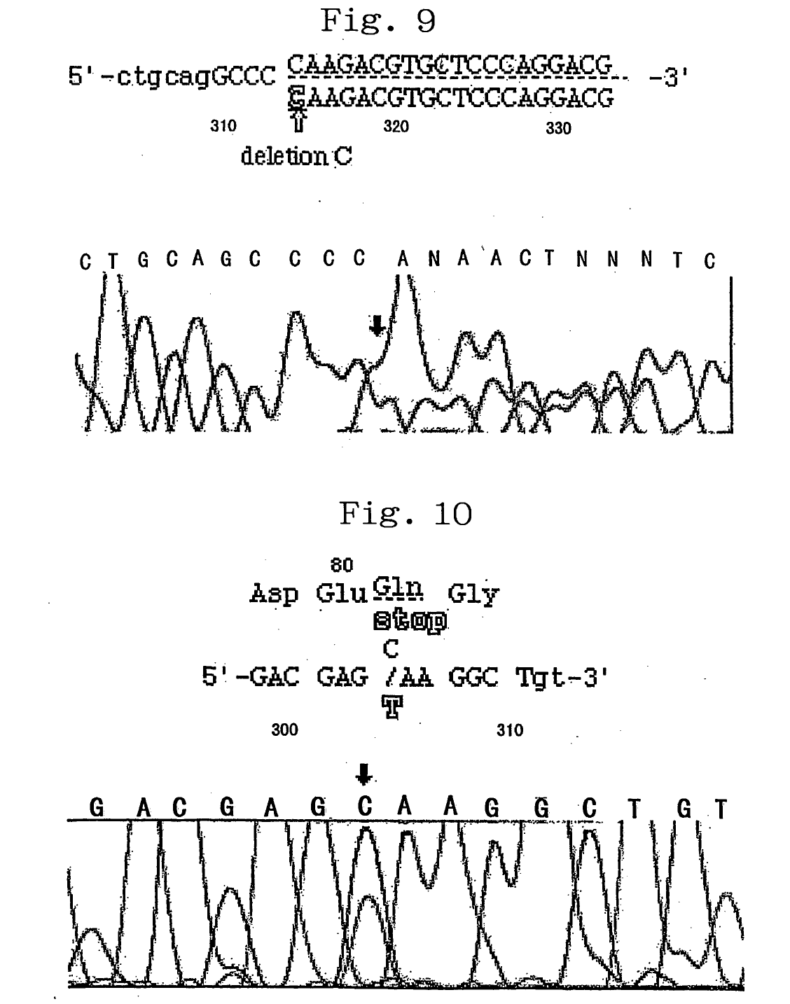

FH case 6 was found to have a loss of a base (C) corresponding to nucleotide 314 in exon 4 of the LDL-R gene, producing a stop codon at the position counting 101 downstream of the codon 84 of the site of deletion (FIG. 9). This deletion mutation anticipates production of an abnormal LDL-R protein consisting of 183 amino acid residues.

Thus, the results have clarified that a risk factor indicating abnormality in lipid metabolism can de detected through correlation between the deletion mutation occurring at nucleotide 314, cytosine, of the LDL-R gene and abnormality in lipid metabolism.

FH case 7 was found to have a substitution C→T at nucleotide 304 (C) in exon 4 of the LDL-R gene, which resulted in a mutation of the LDL-R protein wherein the 81st codon coding for the amino acid residue Gln was changed to a stop codon (FIG. 10). This base substitution anticipates production of an abnormal LDL-R protein consisting of 80 amino acid residues.

These results have clarified that a risk factor indicating abnormality in lipid metabolism can de detected through correlation between the gene mutation occurring at a site coding for amino acid residue 81, glutamine, of the LDL-R protein and abnormality in lipid metabolism.

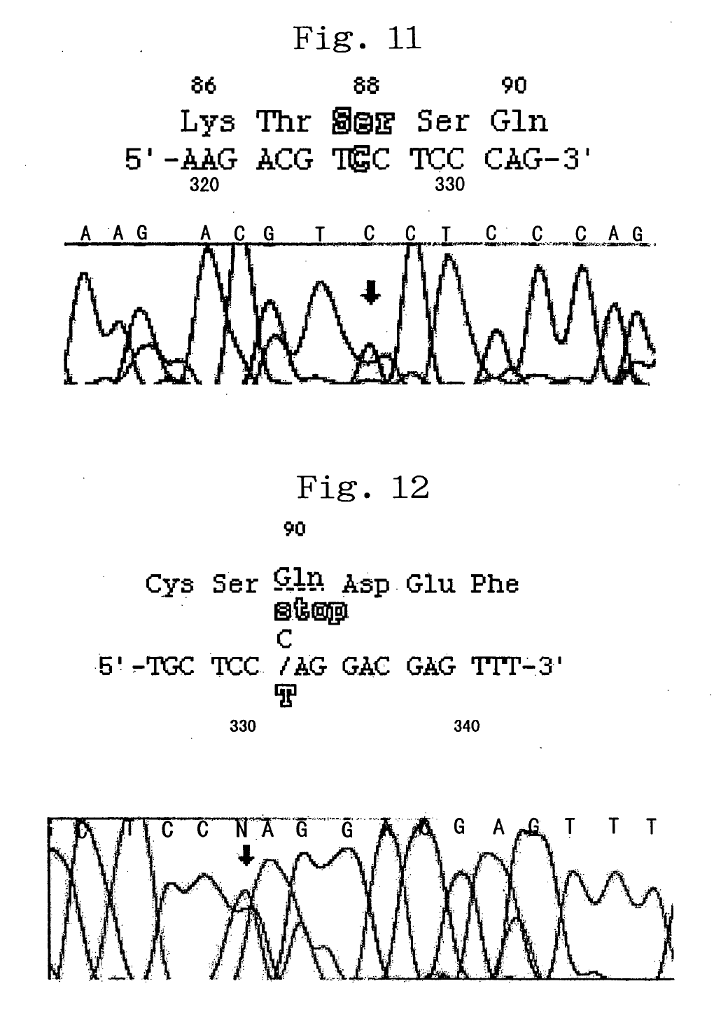

FH case 8 was found to have a substitution G→C at nucleotide 326 (G) in exon 4 of the LDL-R gene, which resulted in a mutation of the LDL-R protein such that the amino acid residue Cys encoded by the 88th codon was replaced by Ser (FIG. 11).

These results have clarified that a risk factor indicating abnormality in lipid metabolism can de detected through correlation between the gene mutation occurring at a site coding for amino acid residue 88, cysteine, of the LDL-R protein and abnormality in lipid metabolism.

FH case 9 was found to have a substitution C→T at nucleotide 331 (C) in exon 4 of the LDL-R gene, which resulted in a mutation of the LDL-R protein wherein the 90th codon coding for amino acid residue Gln was changed to a stop codon (FIG. 12). This base substitution anticipates production of an abnormal LDL-R protein consisting of 89 amino acid residues.

These results have clarified that a risk factor indicating abnormality in lipid metabolism can de detected through correlation between the gene mutation occurring at a site coding for amino acid residue 90, glutamine, of the LDL-R protein and abnormality in lipid metabolism.

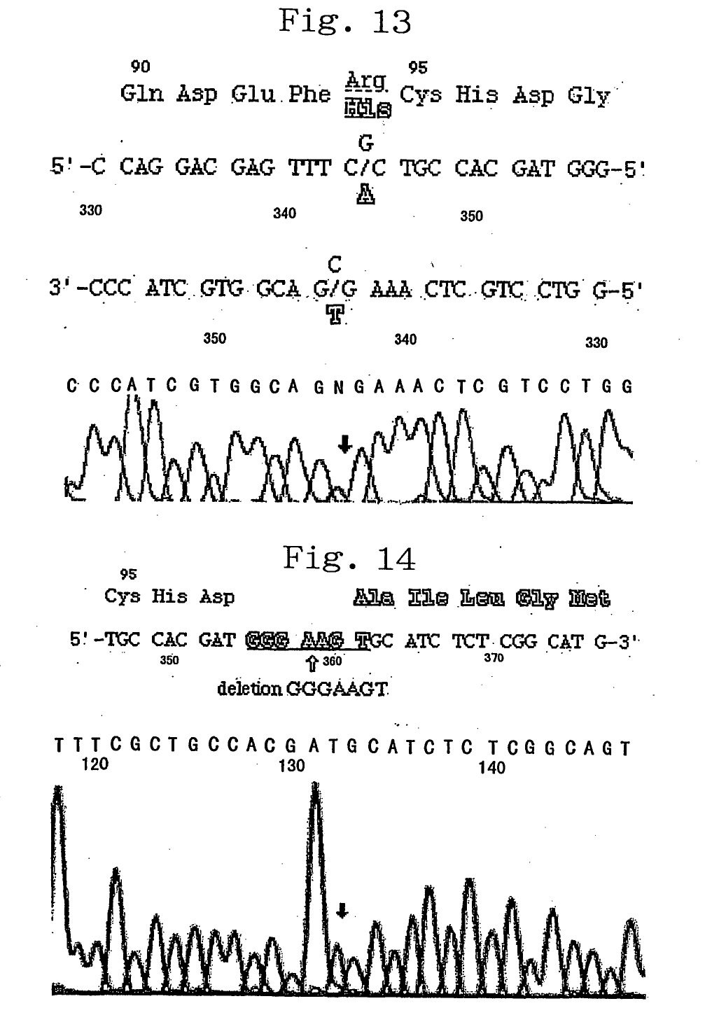

FH case 10 was found to have a substitution G→A at nucleotide 344 (G) in exon 4 of the LDL-R gene, which resulted in a mutation of the LDL-R protein such that the amino acid residue Arg encoded by the 94th codon was replaced by His (FIG. 13).

These results have clarified that a risk factor indicating abnormality in lipid metabolism can de detected through correlation between the gene mutation occurring at a site coding for amino acid residue 94, arginine, of the LDL-R protein and abnormality in lipid metabolism.

FH case 11 was found to have a loss of 7 bases corresponding to nucleotides 355 to 361 (GGGAAGT) in exon 4 of the LDL-R gene, producing a stop codon at the position counting 85 downstream of the codon 98 of the site of deletion (FIG. 14). This deletion mutation anticipates production of an abnormal LDL-R protein consisting of 181 amino acid residues.

Thus, the results have clarified that a risk factor indicating abnormality in lipid metabolism can de detected through correlation between the deletion mutation occurring in nucleotides 355 to 361 of the LDL-R gene and abnormality in lipid metabolism.

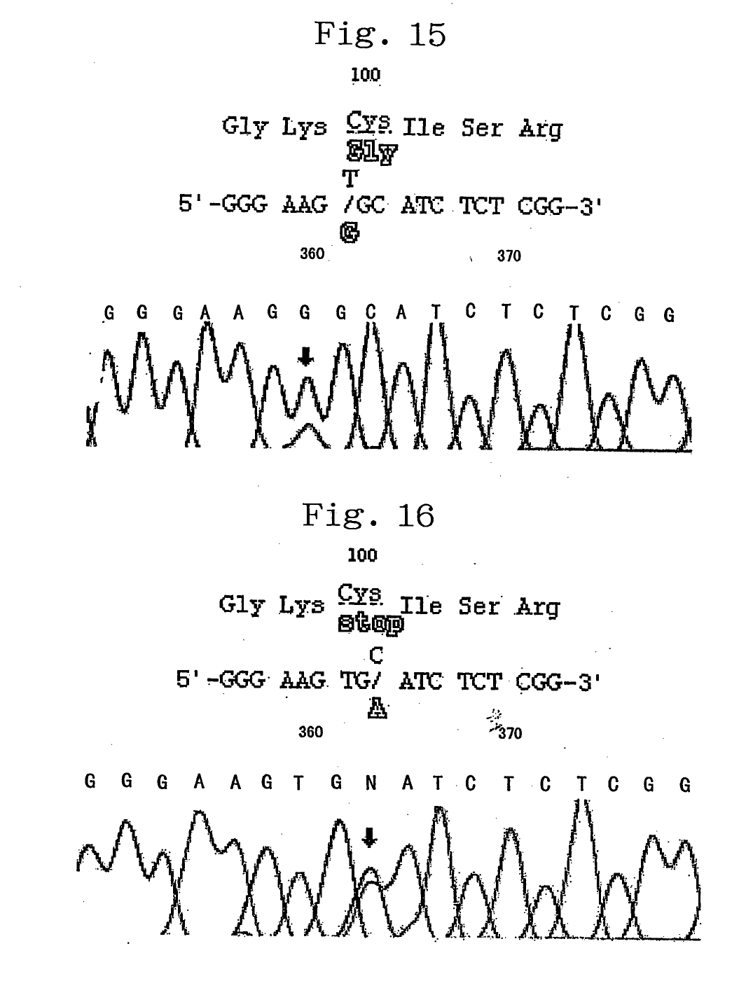

FH case 12 was found to have a substitution T→G at nucleotide 361 (T) in exon 4 of the LDL-R gene, which resulted in a mutation of the LDL-R protein such that the amino acid residue Cys encoded by the 100th codon was replaced by Gly (FIG. 15).

FH case 13 was found to have a substitution C→A at nucleotide 363 (C) in exon 4 of the LDL-R gene, which resulted in a mutation of the LDL-R protein wherein the 100th codon coding for amino acid residue Cys was changed to a stop codon (FIG. 16). This base substitution anticipates production of an abnormal LDL-R protein consisting of 99 amino acid residues.

The results from FH cases 12 and 13 have clarified that a risk factor indicating abnormality in lipid metabolism can de detected through correlation between the gene mutation occurring at a site coding for amino acid residue 100, cysteine, of the LDL-R protein and abnormality in lipid metabolism.

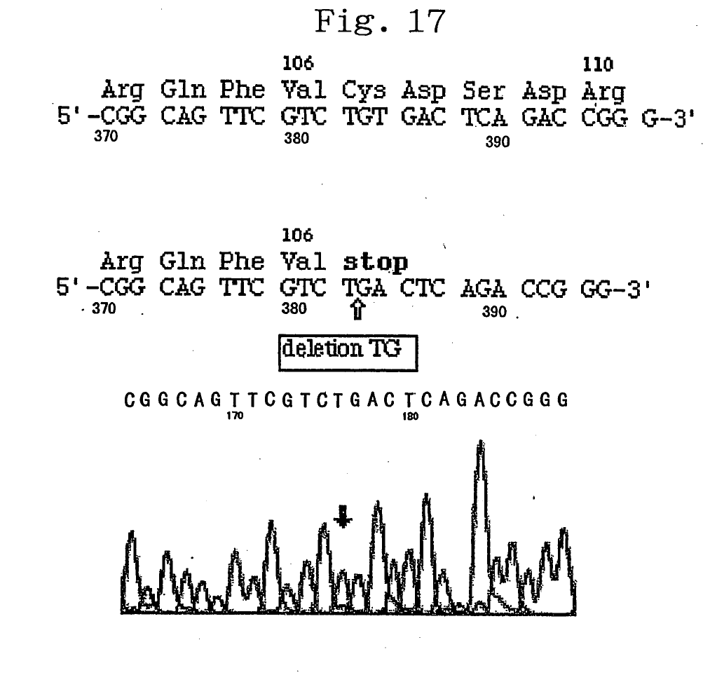

FH case 14 was found to have a loss of two bases (TG) corresponding to nucleotides 382 and 383 in exon 4 of the LDL-R gene, with a codon coding for amino acid residue 107, Cys, having being replaced by a stop codon (FIG. 17). This deletion mutation anticipates production of an abnormal LDL-R protein consisting of 106 amino acid residues.

Thus, the results have clarified that a risk factor indicating abnormality in lipid metabolism can de detected through correlation between the deletion mutation occurring in nucleotides 382 and 383 of the LDL-R gene and abnormality in lipid metabolism.

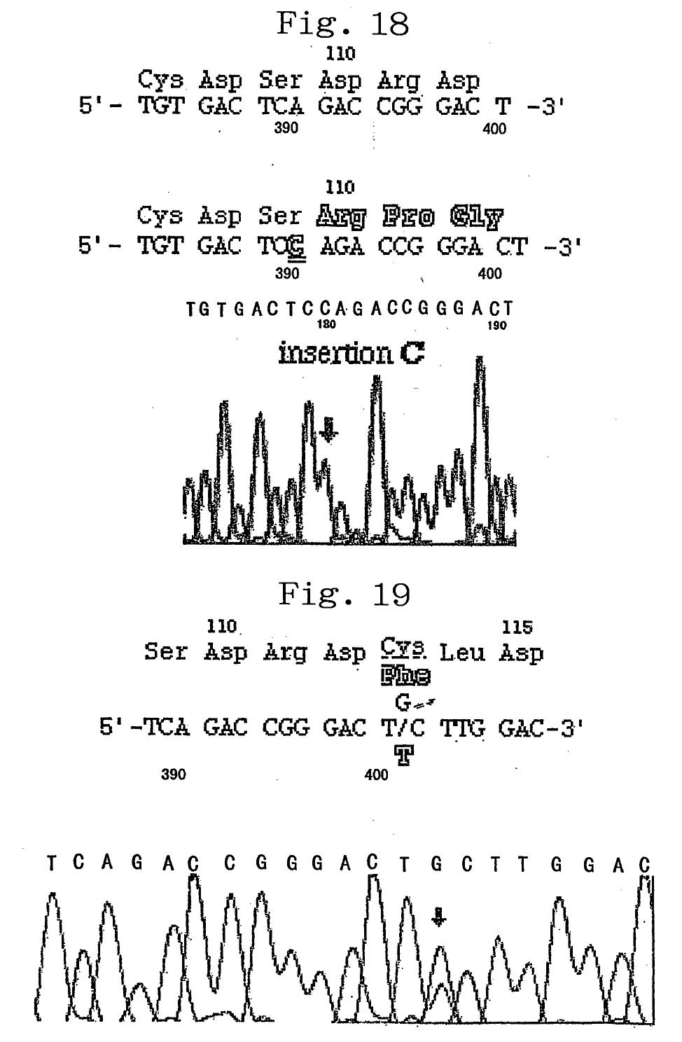

FH case 15 was found to have an insertion of a base (C) at nucleotide 390 in exon 4 of the LDL-R gene, producing a stop codon at the position counting 49 downstream of the codon 110 of the site of insertion (FIG. 18). This insertion mutation anticipates production of an abnormal LDL-R protein consisting of 157 amino acid residues.

Thus, the results have clarified that a risk factor indicating abnormality in lipid metabolism can de detected through correlation between the insertion mutation occurring at a position corresponding to nucleotide 390 of the LDL-R gene and abnormality in lipid metabolism.

FH case 16 was found to have a substitution G→T at nucleotide 401 (G) in exon 4 of the LDL-R gene, which resulted in a mutation of the LDL-R protein such that the amino acid residue Cys encoded by the 113rd codon was replaced by Phe (FIG. 19).

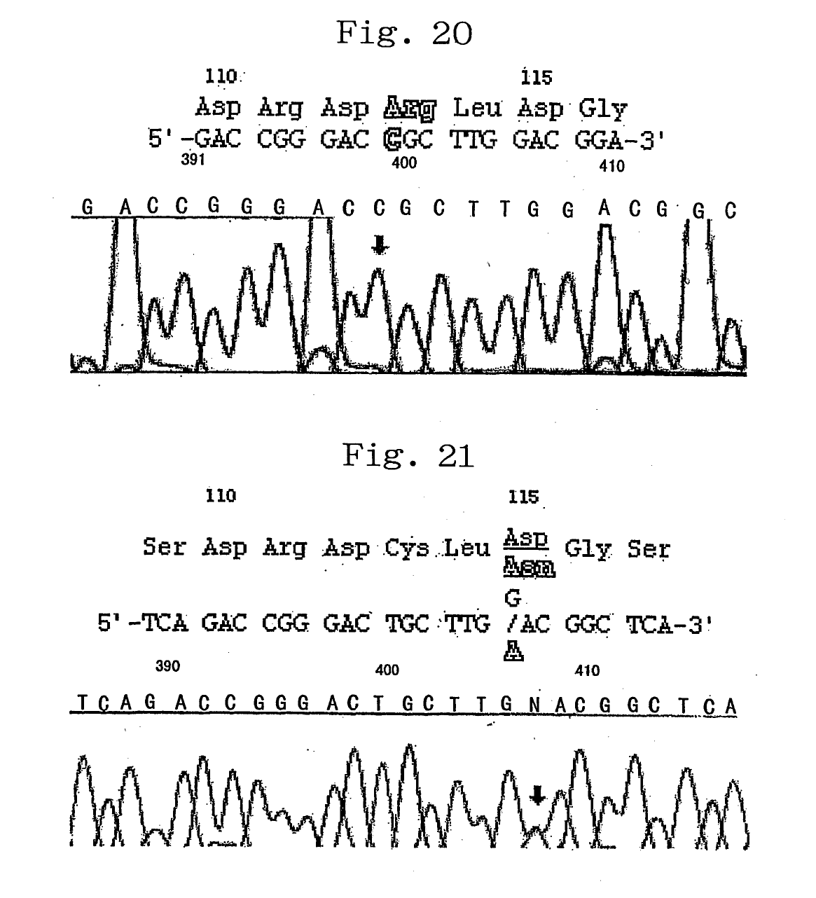

FH case 17 was found to have a substitution T→C at nucleotide 400 (T) in exon 4 of the LDL-R gene, which resulted in a mutation of the LDL-R protein such that the amino acid residue Cys encoded by the 113rd codon was replaced by Arg (FIG. 20).

The results from FH cases 16 and 17 have clarified that a risk factor indicating abnormality in lipid metabolism can de detected through correlation between the gene mutation occurring at a site coding for amino acid residue 113, cysteine, of the LDL-R protein and abnormality in lipid metabolism.

FH case 18 was found to have a substitution G→A at nucleotide 406 (G) in exon 4 of the LDL-R gene, which resulted in a mutation of the LDL-R protein such that the amino acid residue Asp encoded by the 115th codon was replaced by Asn (FIG. 21).

These results have clarified that a risk factor indicating abnormality in lipid metabolism can de detected through correlation between the gene mutation occurring at a site coding for amino acid residue 115, aspartic acid, of the LDL-R protein and abnormality in lipid metabolism.

FH case 19 was found to have a substitution G→A at nucleotide 418 (G) in exon 4 of the LDL-R gene, which resulted in a mutation of the LDL-R protein such that the amino acid residue Glu encoded by the 119th codon was replaced by Lys.

These results have clarified that a risk factor indicating abnormality in lipid metabolism can de detected through correlation between the gene mutation occurring at a site coding for amino acid residue 119, glutamic acid, of the LDL-R protein and abnormality in lipid metabolism.

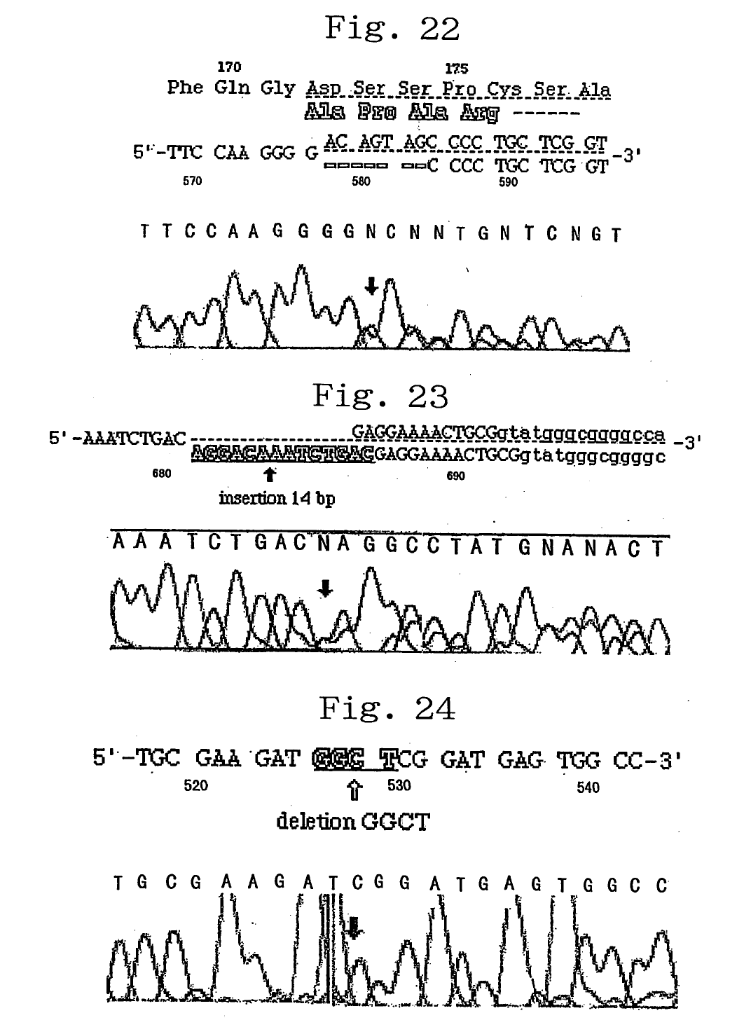

FH case 20 was found to have a loss of 7 bases corresponding to nucleotides 578 to 584 (ACAGTAG) in exon 4 of the LDL-R gene, producing a stop codon at the position counting 11 downstream of the codon 172 of the site of deletion (FIG. 22). This deletion mutation anticipates production of an abnormal LDL-R protein consisting of 180 amino acid residues.

Thus, the results have clarified that a risk factor indicating abnormality in lipid metabolism can be detected through correlation between the deletion mutation occurring in nucleotides 578 to 584 of the LDL-R gene and abnormality in lipid metabolism.

FH case 21 was found to have an insertion of 14 bases (AGGACAAATCTGAC) at nucleotide 682 in exon 4 of the LDL-R gene, producing a stop codon at the position counting 147 downstream of the codon 207 of the site of insertion (FIG. 23). This insertion mutation anticipates production of an abnormal LDL-R protein consisting of 352 amino acid residues.

Thus, the results have clarified that a risk factor indicating abnormality in lipid metabolism can be detected through correlation between the insertion mutation occurring at a position corresponding to nucleotide 682 of the LDL-R gene and abnormality in lipid metabolism.

FH case 22 was found to have a loss of 4 bases corresponding to nucleotides 526 to 529 (GGCT) in exon 4 of the LDL-R gene, producing a stop codon at the position counting 30 downstream of the codon 155 of the site of deletion (FIG. 24). This deletion mutation anticipates production of an abnormal LDL-R protein consisting of 182 amino acid residues.

Thus, the results have clarified that a risk factor indicating abnormality in lipid metabolism can be detected through correlation between the deletion mutation occurring in nucleotides 526 to 529 of the LDL-R gene and abnormality in lipid metabolism.

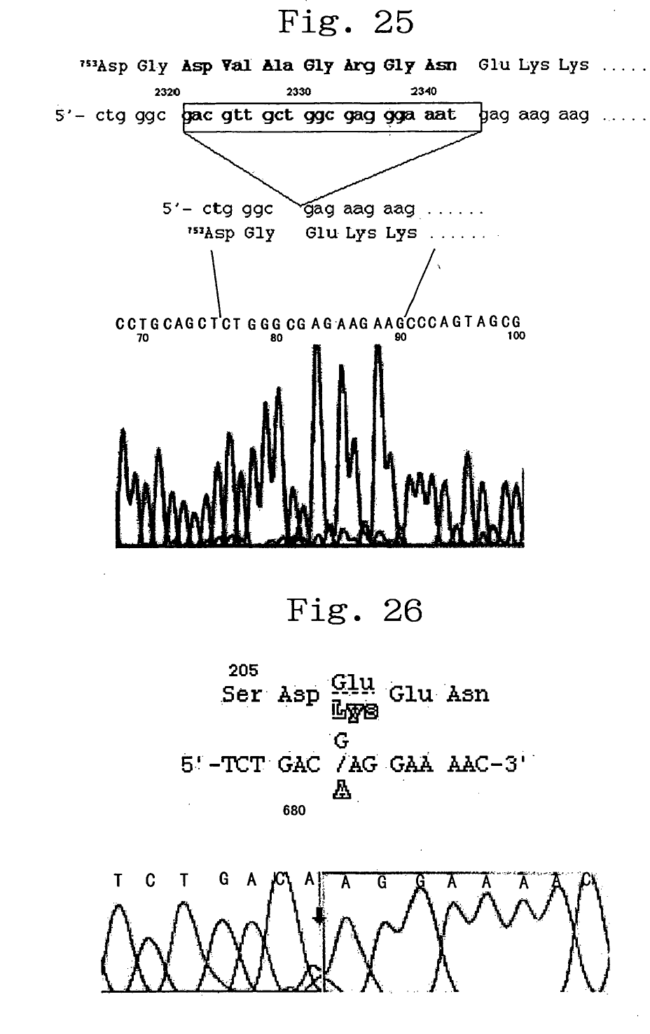

FH case 23 was found to have an insertion of 21 bases (GACTGCAAGGACAAATCTGAC) at nucleotide 661 in exon 4 of the LDL-R gene, and this insertion did not cause a frame shift in codons for amino acid residues (inframe mutation of 21 bases; FIG. 25). The present 21-base insertion was a repetition of seven amino acid residues AspCysLysAspLysSerAsp encoded by 200th to 206th codons. This insertion mutation anticipates production of an abnormal LDL-R protein composed of amino acid residues in a number 7 greater than those constituting a native LDL-R protein.

Thus, the results have clarified that a risk factor indicating abnormality in lipid metabolism can be detected through correlation between the insertion mutation occurring at a position corresponding to nucleotide 661 of the LDL-R gene and abnormality in lipid metabolism.

FH case 24 was found to have a substitution G→A at nucleotide 682 (G) in exon 4 of the LDL-R gene, which resulted in a mutation of the LDL-R protein such that the amino acid residue Glu encoded by the 207th codon was replaced by Lys (FIG. 26).

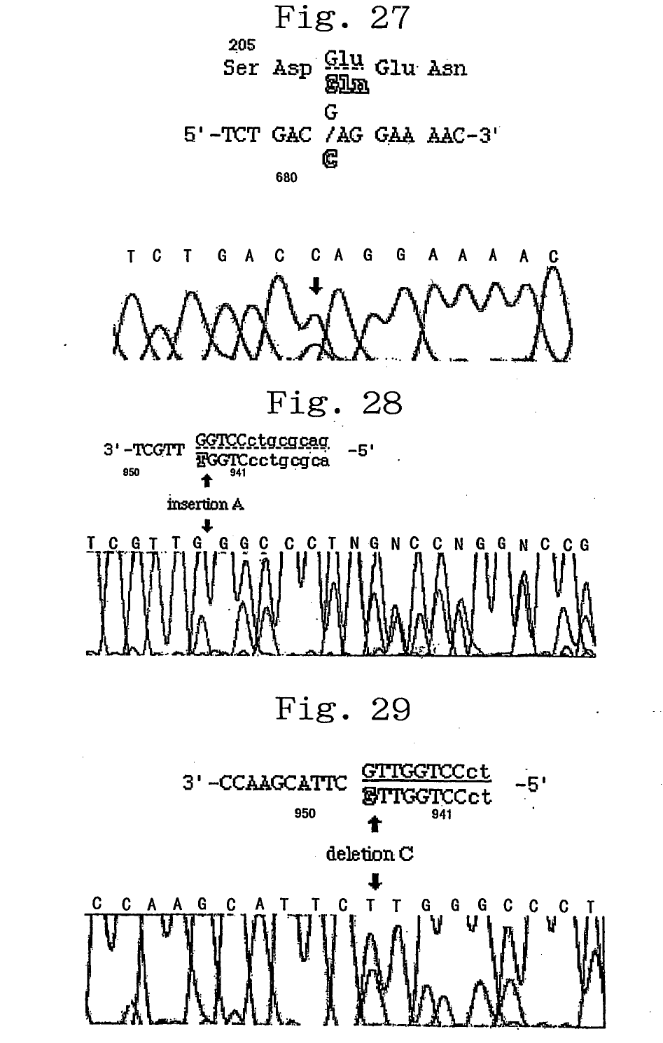

FH case 25 was found to have a substitution G→C at nucleotide 682 (G) in exon 4 of the LDL-R gene, which resulted in a mutation of the LDL-R protein such that the amino acid residue Glu encoded by the 207th codon was replaced by Gln (FIG. 27).

The results from FH cases 24 and 25 have clarified that a risk factor indicating abnormality in lipid metabolism can de detected through correlation between the gene mutation occurring at a site coding for amino acid residue 207, glutamic acid, of the LDL-R protein and abnormality in lipid metabolism.

FH case 26 was found to have an insertion of a base (A) at nucleotide 944 in exon 7 of the LDL-R gene, producing a stop codon at the position counting 17 downstream of the codon 294 of the site of insertion (FIG. 28). This insertion mutation anticipates production of an abnormal LDL-R protein consisting of 309 amino acid residues.

Thus, the results have clarified that a risk factor indicating abnormality in lipid metabolism can be detected through correlation between the insertion mutation occurring at a position corresponding to nucleotide 944 of the LDL-R gene and abnormality in lipid metabolism.

FH case 27 was found to have a loss of a base (C) corresponding to nucleotide 948 in exon 7 of the LDL-R gene, producing a stop codon at the position counting 53 downstream of the codon of the site of deletion (FIG. 29). This deletion mutation anticipates production of an abnormal LDL-R protein consisting of 334 amino acid residues.

Thus, the results have clarified that a risk factor indicating abnormality in lipid metabolism can be detected through correlation between the deletion mutation occurring at nucleotide 948, cytosine, of the LDL-R gene and abnormality in lipid metabolism.

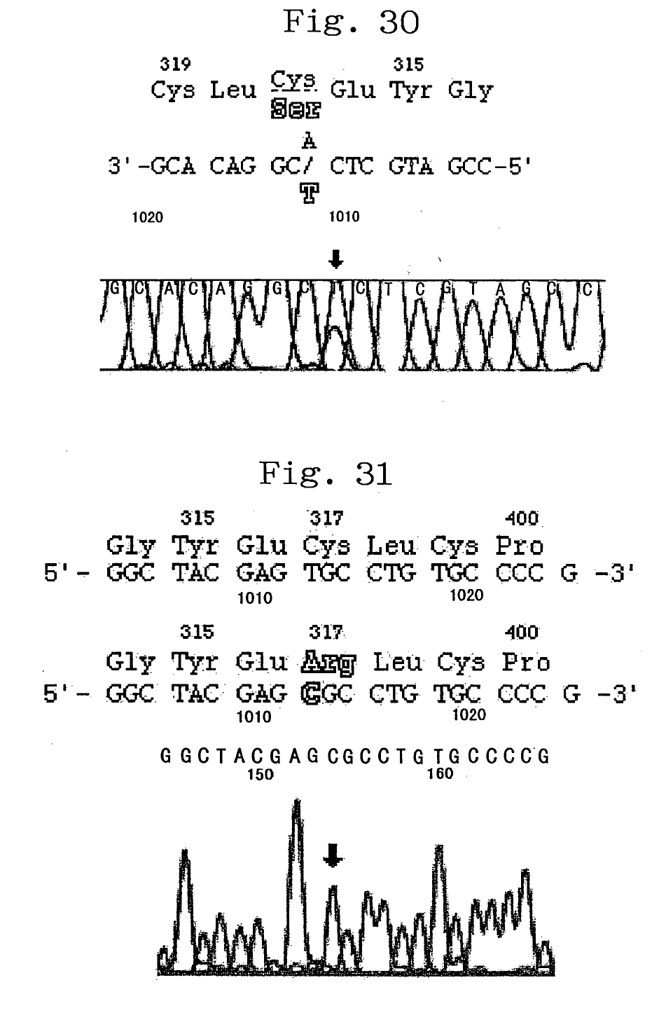

FH case 28 was found to have a substitution T→A at nucleotide 1012 (T) in exon 7 of the LDL-R gene, which resulted in a mutation of the LDL-R protein such that the amino acid residue Cys encoded by the 317th codon was replaced by Ser (FIG. 30).

FH case 29 was found to have a substitution T→C at nucleotide 1012 (T) in exon 7 of the LDL-R gene, which resulted in a mutation of the LDL-R protein such that the amino acid residue Cys encoded by the 317th codon was replaced by Arg (FIG. 31).

The results from FH cases 28 and 29 have clarified that a risk factor indicating abnormality in lipid metabolism can de detected through correlation between the gene mutation occurring at a site coding for amino acid residue 317, cysteine, of the LDL-R protein and abnormality in lipid metabolism.

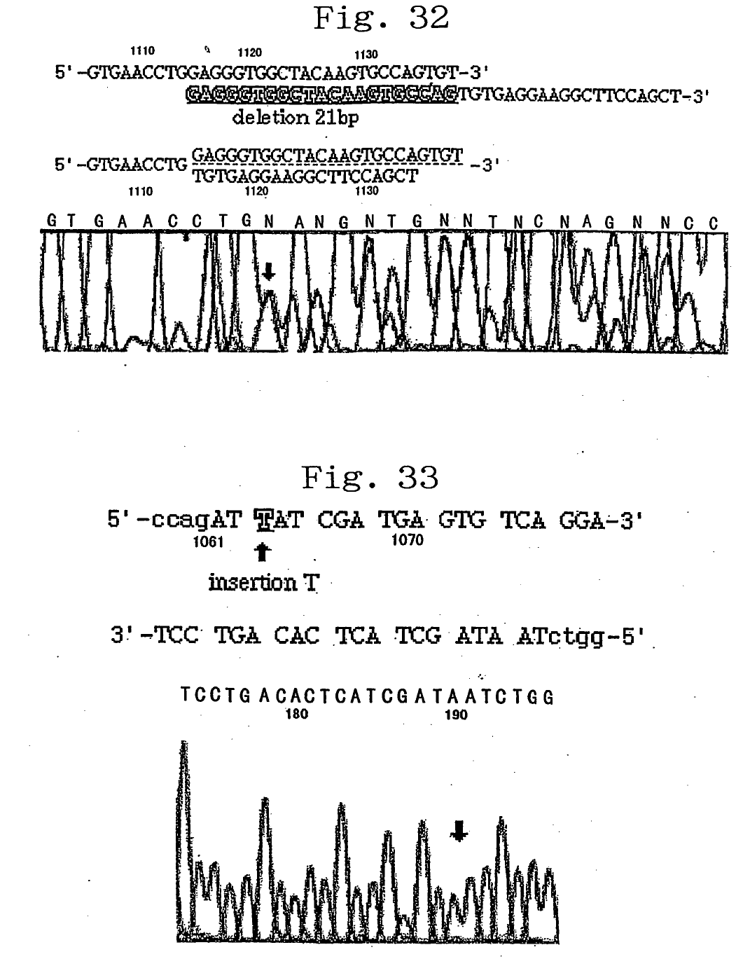

FH case 30 was found to have a loss of 21 bases corresponding to nucleotides 1114 to 1134 in exon 8 of the LDL-R gene, without producing a frame shift, resulting in a deletion mutation of 21 bases (FIG. 32). This deletion mutation anticipates production of an abnormal LDL-R protein lacking the amino acid residues Glu-Gly-Gly-Tyr-Lys-Cys-Gln encoded by the 351st to 357th codons as compared with normal LDL-R protein.

Thus, the results have clarified that a risk factor indicating abnormality in lipid metabolism can be detected through correlation between the deletion mutation occurring in nucleotides 1114 to 1134 of the LDL-R gene and abnormality in lipid metabolism.

FH case 31 was found to have an insertion of a base (T) at nucleotide 1062 in exon 8 of the LDL-R gene, producing a stop codon at the position counting 3 downstream of the codon 333 of the site of insertion (FIG. 33). This insertion mutation anticipates production of an abnormal LDL-R protein consisting of 334 amino acid residues.

Thus, the results have clarified that a risk factor indicating abnormality in lipid metabolism can be detected through correlation between the insertion mutation occurring at a position corresponding to nucleotide 1062 of the LDL-R gene and abnormality in lipid metabolism.

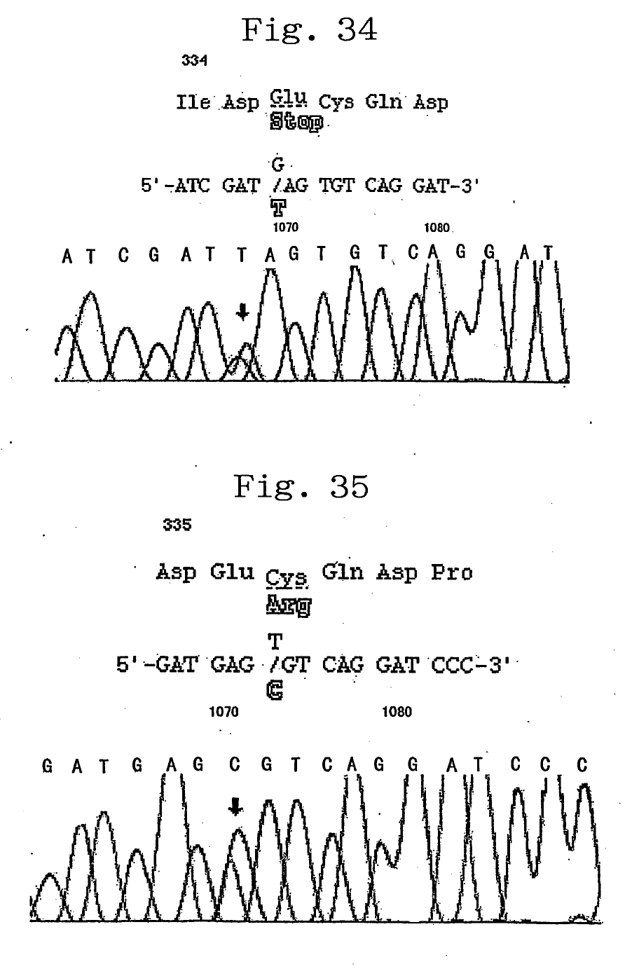

FH case 32 was found to have a substitution G→T at nucleotide 1069 (G) in exon 8 of the LDL-R gene, which resulted in a mutation of the LDL-R protein wherein the 336th codon coding for the amino acid residue Glu was changed to a stop codon (FIG. 34). This base substitution anticipates production of an abnormal LDL-R protein consisting of 335 amino acid residues.

These results have clarified that a risk factor indicating abnormality in lipid metabolism can be detected through correlation between the gene mutation occurring at a site coding for amino acid residue 336, glutamic acid, of the LDL-R protein encoded by the LDL-R gene and abnormality in lipid metabolism.

FH case 33 was found to have a substitution T→C at nucleotide 1072 (T) in exon 8 of the LDL-R gene, which resulted in a mutation of the LDL-R protein such that the amino acid residue Cys encoded by the 337th codon was replaced by Arg (FIG. 35).

These results have clarified that a risk factor indicating abnormality in lipid metabolism can be detected through correlation between the gene mutation occurring at a site coding for amino acid residue 337, cysteine, of the LDL-R protein encoded by the LDL-R gene and abnormality in lipid metabolism.

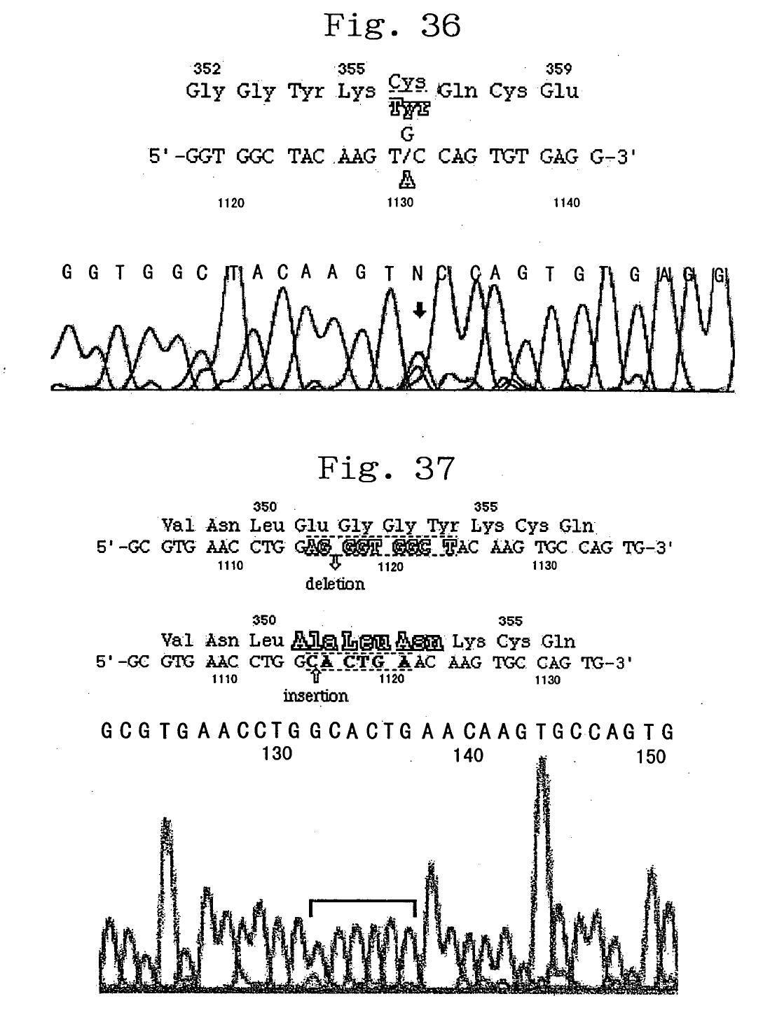

FH case 34 was found to have a substitution G→A at nucleotide 1130 (G) in exon 8 of the LDL-R gene, which resulted in a mutation of the LDL-R protein such that the amino acid residue Cys encoded by the 356th codon was replaced by Tyr (FIG. 36).

These results have clarified that a risk factor indicating abnormality in lipid metabolism can be detected through correlation between the gene mutation occurring at a site coding for amino acid residue 356, cysteine, of the LDL-R protein encoded by the LDL-R gene and abnormality in lipid metabolism.

FH case 35 was found to have a loss of 9 bases corresponding to nucleotides 1115 to 1223 (AGGGTGGCT) and an insertion of 6 bases (CACTGA) in exon 8 of the LDL-R gene (FIG. 37), without producing a frame shift, resulting in a mutation of the LDL-R protein such that the amino acid sequence Glu-Gly-Gly-Tyr encoded by the 351st to 354th codons was replaced by a sequence Ala-Leu-Asn. This mutation anticipates production of an abnormal LDL-R protein lacking one amino acid residue as compared with the normal LDL-R protein.

These results have clarified that a risk factor indicating abnormality in lipid metabolism can be detected through correlation between the gene mutation occurring at a site coding for amino acid sequence 351 to 354, glutamic acid—glycine—glycine—tyrosine, of the LDL-R protein encoded by the LDL-R gene and abnormality in lipid metabolism.

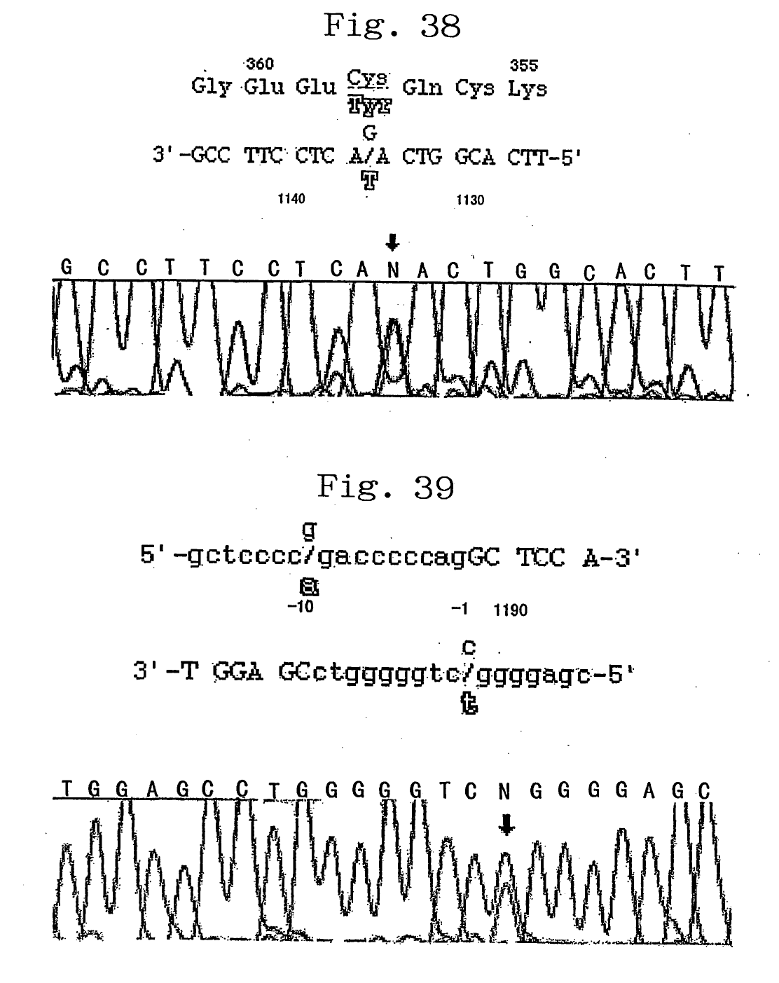

FH case 36 was found to have a substitution G→A at nucleotide 1136 (G) in exon 8 of the LDL-R gene, which resulted in a mutation of the LDL-R protein such that the amino acid residue Cys encoded by the 358th codon was replaced by Tyr (FIG. 38).

These results have clarified that a risk factor indicating abnormality in lipid metabolism can be detected through correlation between the gene mutation occurring at a site coding for amino acid residue 358, cysteine, of the LDL-R protein encoded by the LDL-R gene and abnormality in lipid metabolism.

FH case 37 was found to have a substitution G→A at nucleotide −10 (G) in the 5′-end-side acceptor region in intron 8 of the LDL-R gene (FIG. 39). This region is considered to play an important role in the formation of a lariat structure of mRNA during translation of the mRNA.

These results have clarified that a risk factor indicating abnormality in lipid metabolism can be detected through correlation between the mutation of guanine at nucleotide −10 (G) in the 5′-end-side acceptor region in intron 8 of the LDL-R gene and abnormality in lipid metabolism.

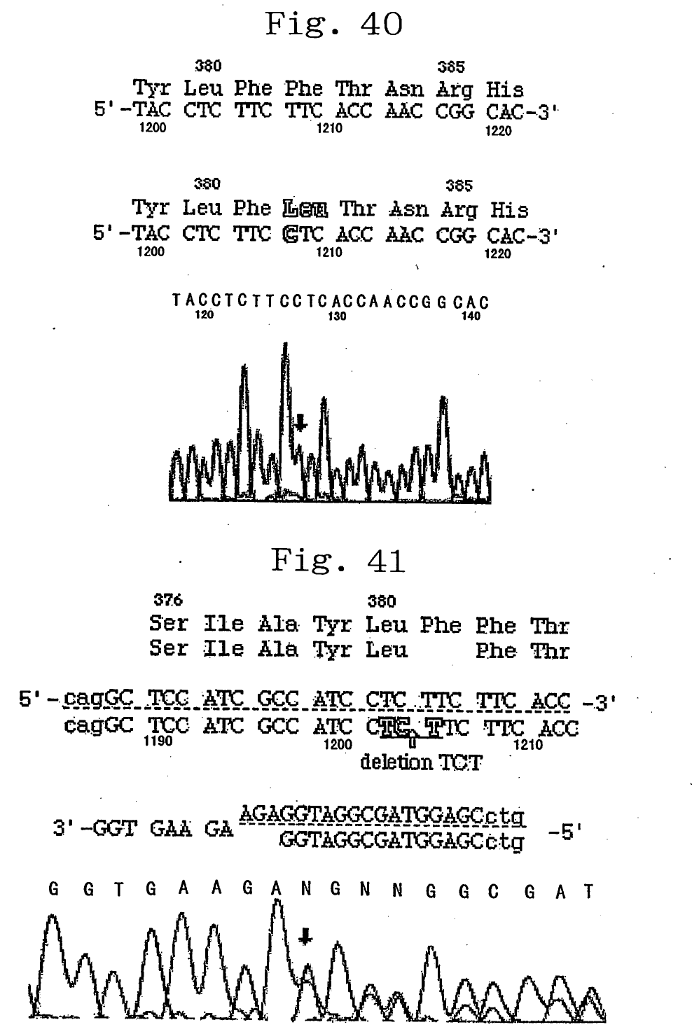

FH case 38 was found to have a substitution T→C at nucleotide 1207 (T) in exon 9 of the LDL-R gene, which resulted in a mutation of the LDL-R protein such that the amino acid residue Phe encoded by the 382nd codon was replaced by Leu (FIG. 40).

These results have clarified that a risk factor indicating abnormality in lipid metabolism can be detected through correlation between the gene mutation occurring at a site coding for amino acid residue 382, phenylalanine, of the LDL-R protein encoded by the LDL-R gene and abnormality in lipid metabolism.

FH case 39 was found to have a loss of 3 bases (TCT) corresponding to nucleotides 1202 to 1204 in exon 9 of the LDL-R gene, without producing a frame shift, which resulted in a mutation of the LDL-R protein such that the amino acid residue Phe encoded by the 381st codon was deleted (FIG. 41). This deletion mutation anticipates production of an abnormal LDL-R protein lacking one amino acid residue as compared with the normal LDL-R protein.

Thus, the results have clarified that a risk factor indicating abnormality in lipid metabolism can be detected through correlation between the deletion mutation occurring in nucleotides 1202 to 1204 of the LDL-R gene and abnormality in lipid metabolism.

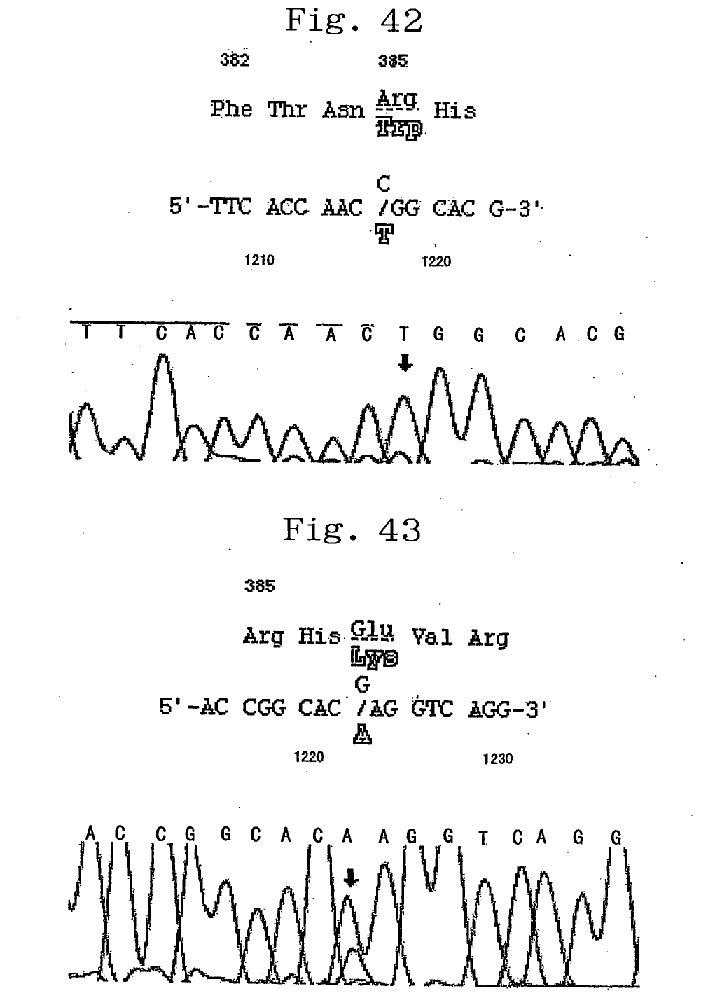

FH case 40 was found to have a substitution C→T at nucleotide 1216 (C) in exon 9 of the LDL-R gene, which resulted in a mutation of the LDL-R protein such that the amino acid residue Arg encoded by the 385th codon was replaced by Trp (FIG. 42).

These results have clarified that a risk factor indicating abnormality in lipid metabolism can be detected through correlation between the gene mutation occurring at a site coding for amino acid residue 385, arginine, of the LDL-R protein encoded by the LDL-R gene and abnormality in lipid metabolism.

FH case 41 was found to have a substitution G→A at nucleotide 1222 (G) in exon 9 of the LDL-R gene, which resulted in a mutation of the LDL-R protein such that the amino acid residue Glu encoded by the 387th codon was replaced by Lys (FIG. 43).

These results have clarified that a risk factor indicating abnormality in lipid metabolism can be detected through correlation between the gene mutation occurring at a site coding for amino acid residue 387, glutamic acid, of the LDL-R protein encoded by the LDL-R gene and abnormality in lipid metabolism.

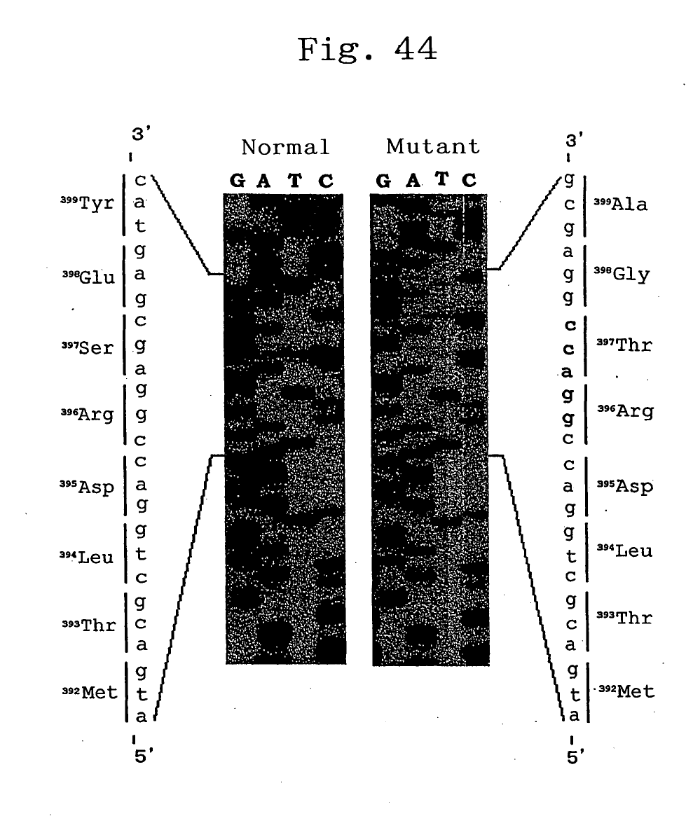

FH case 42 was found to have an insertion of 5 bases corresponding to nucleotides 1242 to 1246 (GGACC) in exon 9 of the LDL-R gene, producing a stop codon at the position counting 15 downstream of the codon 393 of the site of insertion (FIG. 44). This insertion mutation anticipates production of an abnormal LDL-R protein consisting of 407 amino acid residues.

Thus, the results have clarified that a risk factor indicating abnormality in lipid metabolism can be detected through correlation between the insertion mutation occurring at a position corresponding to nucleotide 1242 of the LDL-R gene and abnormality in lipid metabolism.

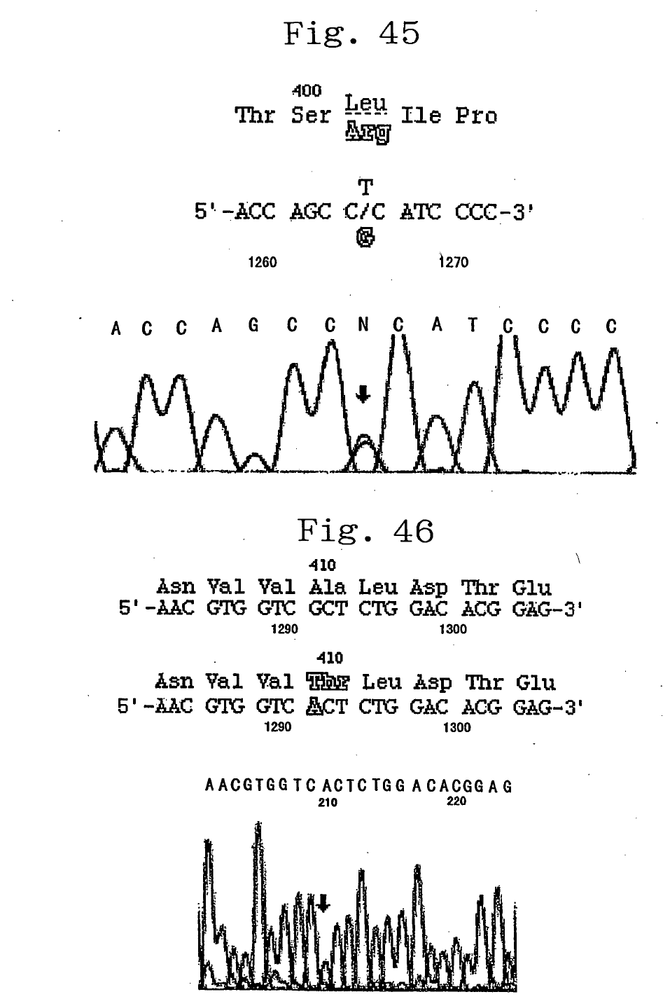

FH case 43 was found to have a substitution T→G at nucleotide 1265 (T) in exon 9 of the LDL-R gene, which resulted in a mutation of the LDL-R protein such that the amino acid residue Leu encoded by the 401st codon was replaced by Arg (FIG. 45).

These results have clarified that a risk factor indicating abnormality in lipid metabolism can be detected through correlation between the gene mutation occurring at a site coding for amino acid residue 401, leucine, of the LDL-R protein encoded by the LDL-R gene and abnormality in lipid metabolism.

FH case 44 was found to have a substitution G→A at nucleotide 1291 (G) in exon 9 of the LDL-R gene, which resulted in a mutation of the LDL-R protein such that the amino acid residue Ala encoded by the 410th codon was replaced by Thr (FIG. 46).

These results have clarified that a risk factor indicating abnormality in lipid metabolism can be detected through correlation between the gene mutation occurring at a site coding for amino acid residue 410, alanine, of the LDL-R protein encoded by the LDL-R gene and abnormality in lipid metabolism.

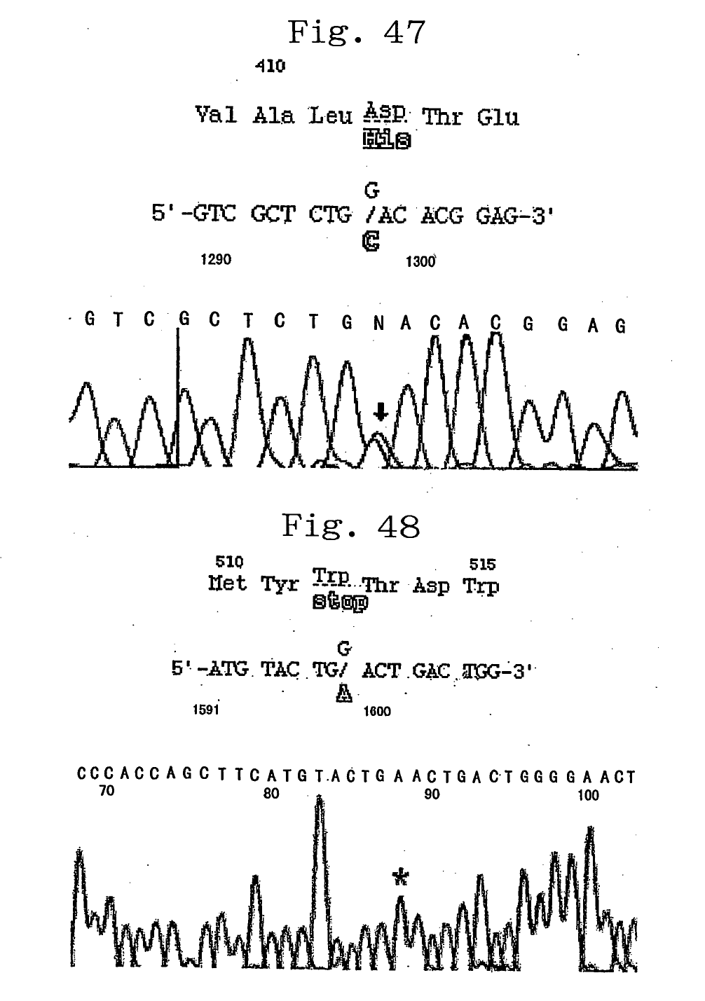

FH case 45 was found to have a substitution G→C at nucleotide 1297 (G) in exon 9 of the LDL-R gene, which resulted in a mutation of the LDL-R protein such that the amino acid residue Asp encoded by the 412nd codon was replaced by His (FIG. 47).

These results have clarified that a risk factor indicating abnormality in lipid metabolism can be detected through correlation between the gene mutation occurring at a site coding for amino acid residue 412, aspartic acid, of the LDL-R protein encoded by the LDL-R gene and abnormality in lipid metabolism.

FH case 46 was found to have a substitution G→A at nucleotide 1599 (G) in exon 11 of the LDL-R gene, which resulted in a mutation of the LDL-R protein such that the amino acid residue Trp encoded by the 512nd codon was replaced by Arg (FIG. 48). This substitution anticipates production of an abnormal LDL-R protein consisting of 511 amino acid residues. These results have clarified that a risk factor indicating abnormality in lipid metabolism can be detected through correlation between the gene mutation occurring at a site coding for amino acid residue 512, tryptophan, of the LDL-R protein encoded by the LDL-R gene and abnormality in lipid metabolism.

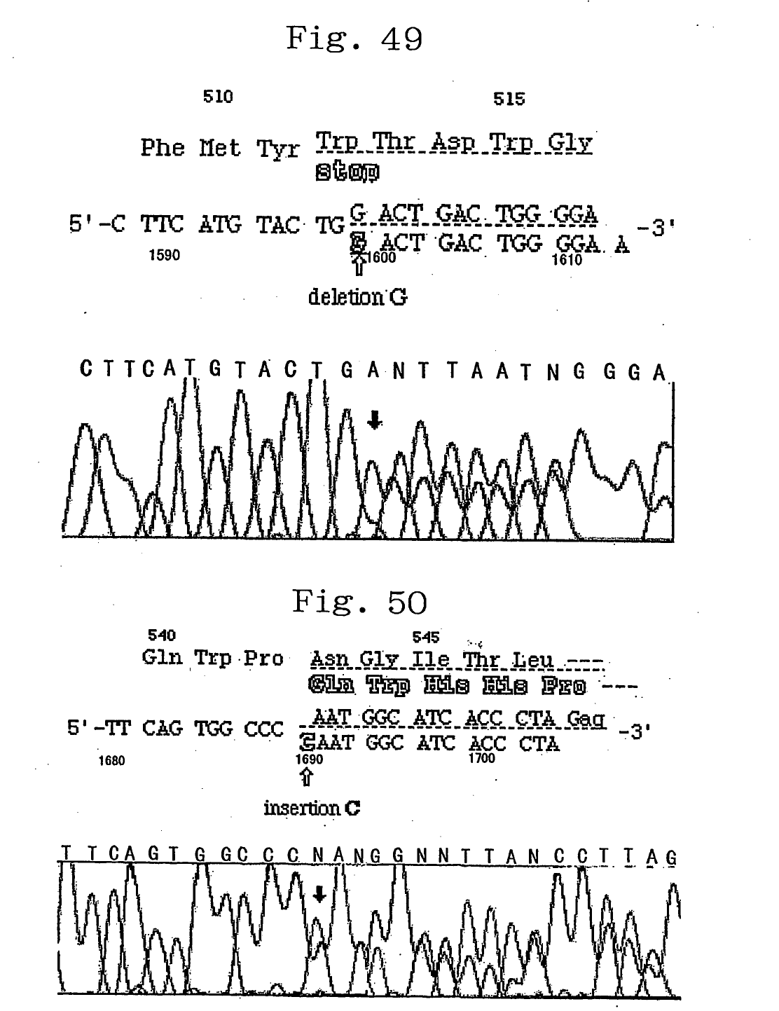

FH case 47 was found to have a loss of a base (G) corresponding to nucleotide 1599 in exon 11 of the LDL-R gene, with a codon coding for amino acid residue 512 having being replaced by a stop codon (FIG. 49). This deletion mutation anticipates production of an abnormal LDL-R protein consisting of 511 amino acid residues.

Thus, the results have clarified that a risk factor indicating abnormality in lipid metabolism can be detected through correlation between the deletion mutation occurring in nucleotide 1599 of the LDL-R gene and abnormality in lipid metabolism.

FH case 48 was found to have an insertion of a base (C) at nucleotide 1687 in exon 11 of the LDL-R gene, producing a stop codon at the position counting 16 downstream of the codon 542 of the site of insertion (FIG. 50). This insertion mutation anticipates production of an abnormal LDL-R protein consisting of 556 amino acid residues.

Thus, the results have clarified that a risk factor indicating abnormality in lipid metabolism can be detected through correlation between the insertion mutation occurring at a position corresponding to nucleotide 1687 of the LDL-R gene and abnormality in lipid metabolism.

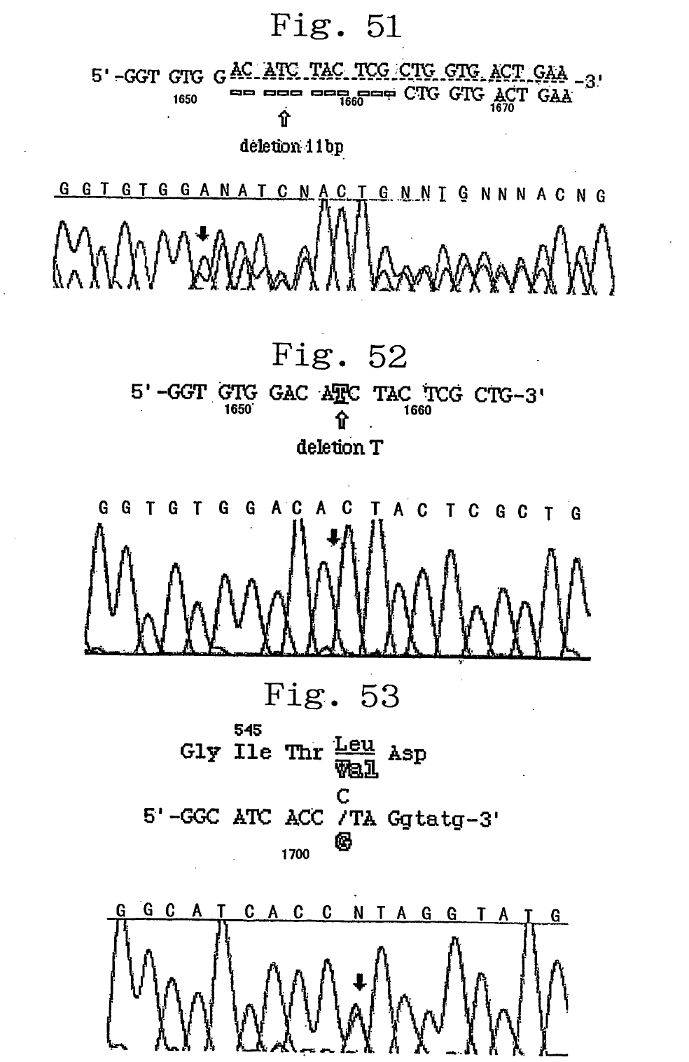

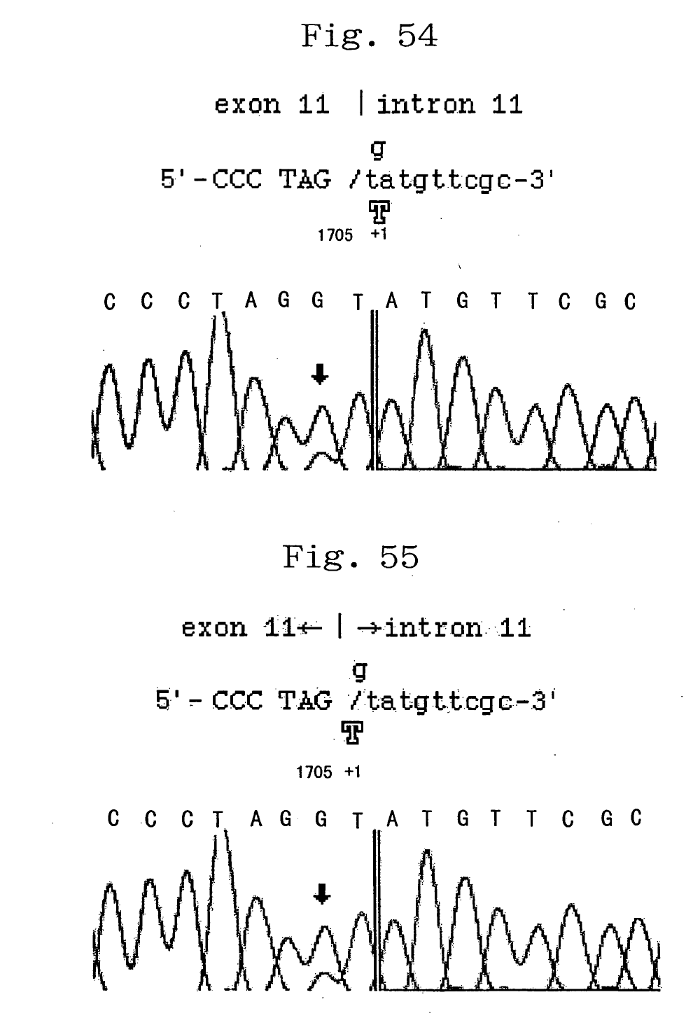

FH case 49 was found to have a loss of 11 bases corresponding to nucleotides 1652 to 1662 (ACATCTACTCG) in exon 11 of the LDL-R gene, producing a stop codon at the position counting 4 downstream of the codon 530 of the site of deletion (FIG. 51). This deletion mutation anticipates production of an abnormal LDL-R protein consisting of 533 amino acid residues.

Thus, the results have clarified that a risk factor indicating abnormality in lipid metabolism can be detected through correlation between the deletion mutation occurring in nucleotides 1652 to 1662 of the LDL-R gene and abnormality in lipid metabolism.

FH case 50 was found to have a loss of a base (T) corresponding to nucleotide 1655 in exon 11 of the LDL-R gene, producing a stop codon at the position counting 28 downstream of the codon 531 of the site of deletion (FIG. 52). This deletion mutation anticipates production of an abnormal LDL-R protein consisting of 557 amino acid residues.

Thus, the results have clarified that a risk factor indicating abnormality in lipid metabolism can be detected through correlation between the deletion mutation occurring at nucleotide 1655, thymine, of the LDL-R gene and abnormality in lipid metabolism.

FH case 51 was found to have a substitution C→G at nucleotide 1702 (C) in exon 11 of the LDL-R gene, which resulted in a mutation of the LDL-R protein such that the amino acid residue Leu encoded by the 547th codon was replaced by Val (FIG. 53).

These results have clarified that a risk factor indicating abnormality in lipid metabolism can be detected through correlation between the gene mutation occurring at a site coding for amino acid residue 547, leucine, of the LDL-R protein encoded by the LDL-R gene and abnormality in lipid metabolism.

FH cases 52 and 53 were found to have substitution mutations G→C and G→T, respectively, at nucleotide +1 (guanine) in a splice donor site of intron 11 of the LDL-R gene, causing abnormal splicing, which resulted in inhibiting normal translation of mRNA. These substitution mutations anticipate production of abnormal mRNA.

These results have clarified that a risk factor indicating abnormality in lipid metabolism can be detected through correlation between abnormality in lipid metabolism and the gene mutation, from guanine to another base, of nucleotide +1 (G) in a splice donor site of intron 11 starting from the nucleotide that is one base downstream the nucleotide 1705 of the LDL-R gene.

FH case 54 was found to have a substitution mutation T→G, at nucleotide +2 (T) in a splice donor site of intron 12 of the LDL-R gene, causing abnormal splicing, which resulted in inhibiting normal translation of mRNA. This substitution mutation anticipates production of abnormal mRNA.

These results have clarified that a risk factor indicating abnormality in lipid metabolism can be detected through correlation between abnormality in lipid metabolism and the gene mutation, from thymine to another base, of nucleotide +2 (T) in a splice donor site of intron 12 starting from the nucleotide that is one base downstream the nucleotide 1845 of the LDL-R gene.

FH case 55 was found to have a substitution G→T at nucleotide 1731 (G) in exon 12 of the LDL-R gene, which resulted in a mutation of the LDL-R protein such that the amino acid residue Trp encoded by the 556th codon was replaced by Cys.

These results have clarified that a risk factor indicating abnormality in lipid metabolism can be detected through correlation between the gene mutation occurring at a site coding for amino acid residue 556, tryptophan, of the LDL-R protein encoded by the LDL-R gene and abnormality in lipid metabolism.

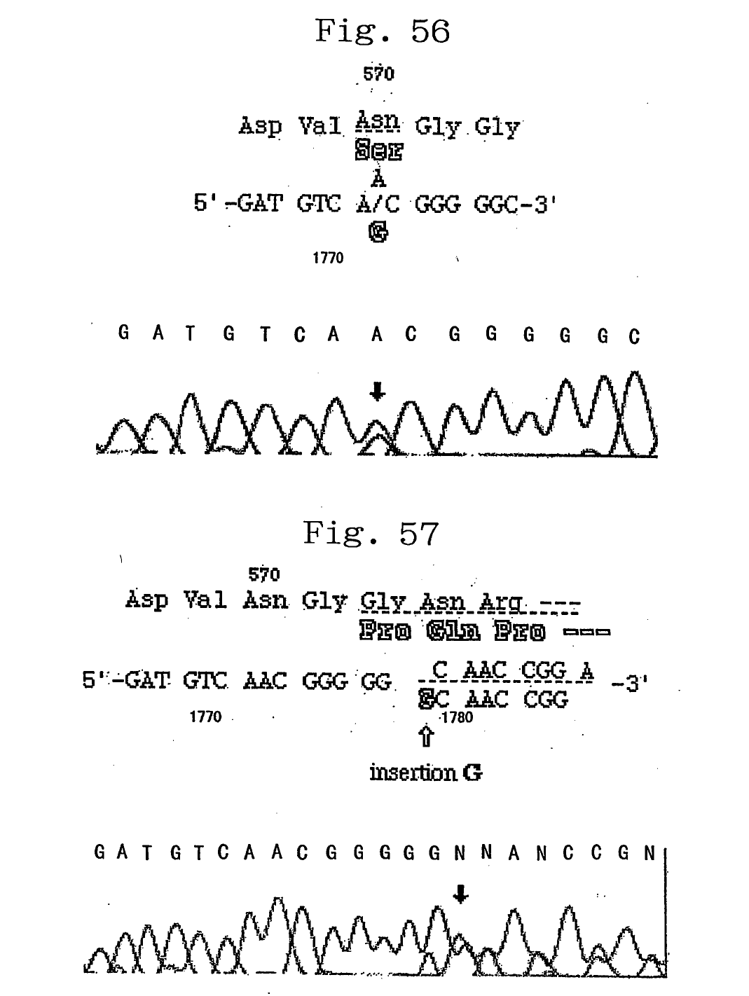

FH case 56 was found to have a substitution A→G at nucleotide 1772 (A) in exon 12 of the LDL-R gene, which resulted in a mutation of the LDL-R protein such that the amino acid residue Asn encoded by the 570th codon was replaced by Ser (FIG. 56).

These results have clarified that a risk factor indicating abnormality in lipid metabolism can be detected through correlation between the gene mutation occurring at a site coding for amino acid residue 570, asparagine, of the LDL-R protein encoded by the LDL-R gene and abnormality in lipid metabolism.

FH case 57 was found to have an insertion of a base (G) at nucleotide 1779 in exon 12 of the LDL-R gene, producing a stop codon at the position counting 10 downstream of the codon 572 of the site of insertion (FIG. 57). This insertion mutation anticipates production of an abnormal LDL-R protein consisting of 580 amino acid residues.

Thus, the results have clarified that a risk factor indicating abnormality in lipid metabolism can be detected through correlation between the insertion mutation occurring at a position corresponding to nucleotide 1779 of the LDL-R gene and abnormality in lipid metabolism.

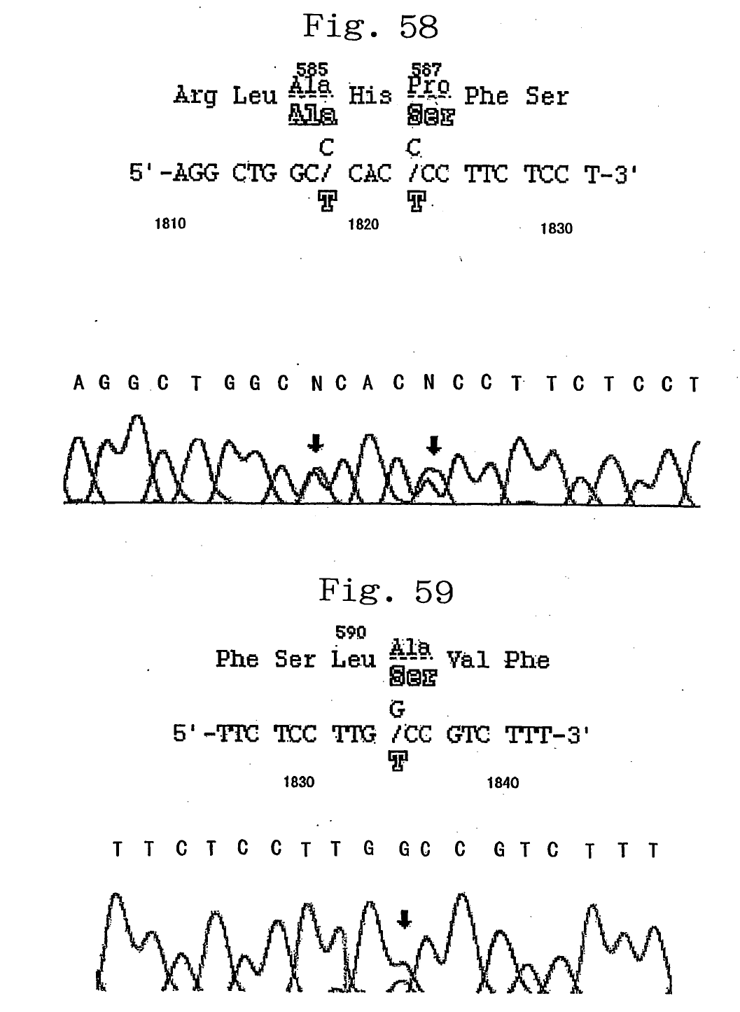

FH case 58 was found to have a substitution C→T at nucleotide 1822 (C) in exon 12 of the LDL-R gene, which resulted in a mutation of the LDL-R protein such that the amino acid residue Pro encoded by the 587th codon was replaced by Ser (FIG. 58).

These results have clarified that a risk factor indicating abnormality in lipid metabolism can be detected through correlation between the gene mutation occurring at a site coding for amino acid residue 587, proline, of the LDL-R protein encoded by the LDL-R gene and abnormality in lipid metabolism.

FH case 59 was found to have a substitution G→T at nucleotide 1834 (G) in exon 12 of the LDL-R gene, which resulted in a mutation of the LDL-R protein such that the amino acid residue Ala encoded by the 591st codon was replaced by Ser (FIG. 59).

These results have clarified that a risk factor indicating abnormality in lipid metabolism can be detected through correlation between the gene mutation occurring at a site coding for amino acid residue 591, alanine, of the LDL-R protein encoded by the LDL-R gene and abnormality in lipid metabolism.

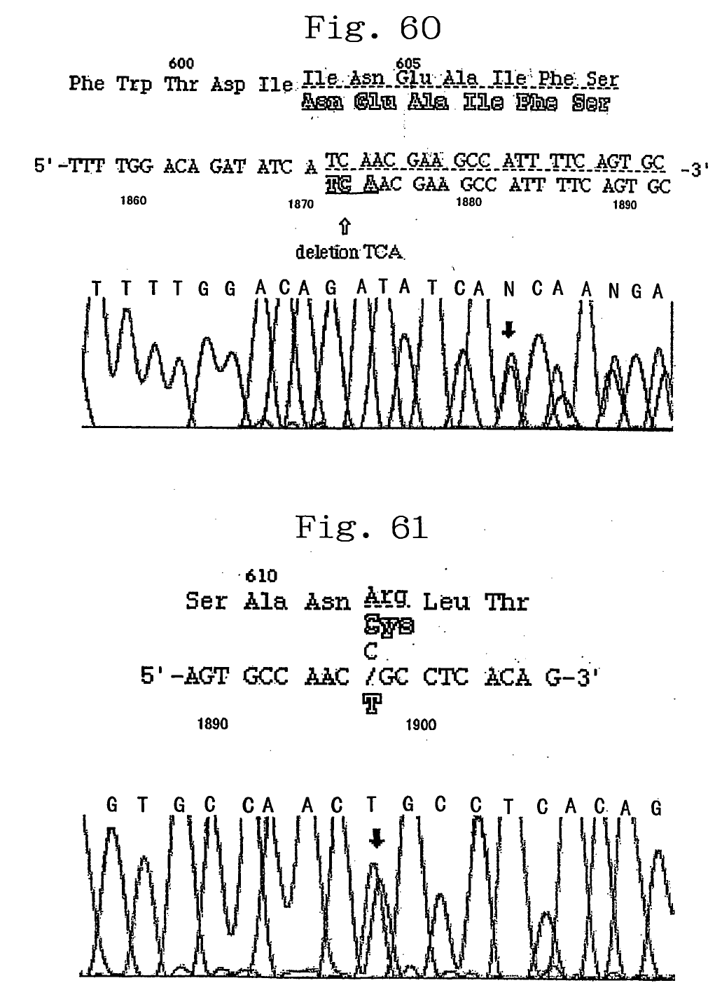

FH case 60 was found to have a loss of 3 bases (TCA) corresponding to nucleotides 1870 to 1872 in exon 13 of the LDL-R gene, without having produced a frame shift, which resulted in a mutation of the LDL-R protein such that the amino acid residue Ile encoded by the 603rd codon was deleted (FIG. 60). This deletion mutation anticipates production of an abnormal LDL-R protein lacking one amino acid residue as compared with the normal LDL-R protein.

Thus, the results have clarified that a risk factor indicating abnormality in lipid metabolism can be detected through correlation between the deletion mutation occurring in nucleotides 1870 to 1872 of the LDL-R gene and abnormality in lipid metabolism.

FH case 61 was found to have a substitution C→T at nucleotide 1897 (C) in exon 13 of the LDL-R gene, which resulted in a mutation of the LDL-R protein such that the amino acid residue Arg encoded by the 612nd codon was replaced by Cys (FIG. 61).

These results have clarified that a risk factor indicating abnormality in lipid metabolism can be detected through correlation between the gene mutation occurring at a site coding for amino acid residue 612, arginine, of the LDL-R protein encoded by the LDL-R gene and abnormality in lipid metabolism.

FH case 62 was found to have a loss of a base corresponding to nucleotide 1963 (T) in exon 13 of the LDL-R gene, producing a stop codon at the position counting 10 downstream of the codon 643 of the site of deletion (FIG. 62). This deletion mutation anticipates production of an abnormal LDL-R protein consisting of 642 amino acid residues.

Thus, the results have clarified that a risk factor indicating abnormality in lipid metabolism can be detected through correlation between the deletion mutation occurring in nucleotide 1963 of the LDL-R gene and abnormality in lipid metabolism.

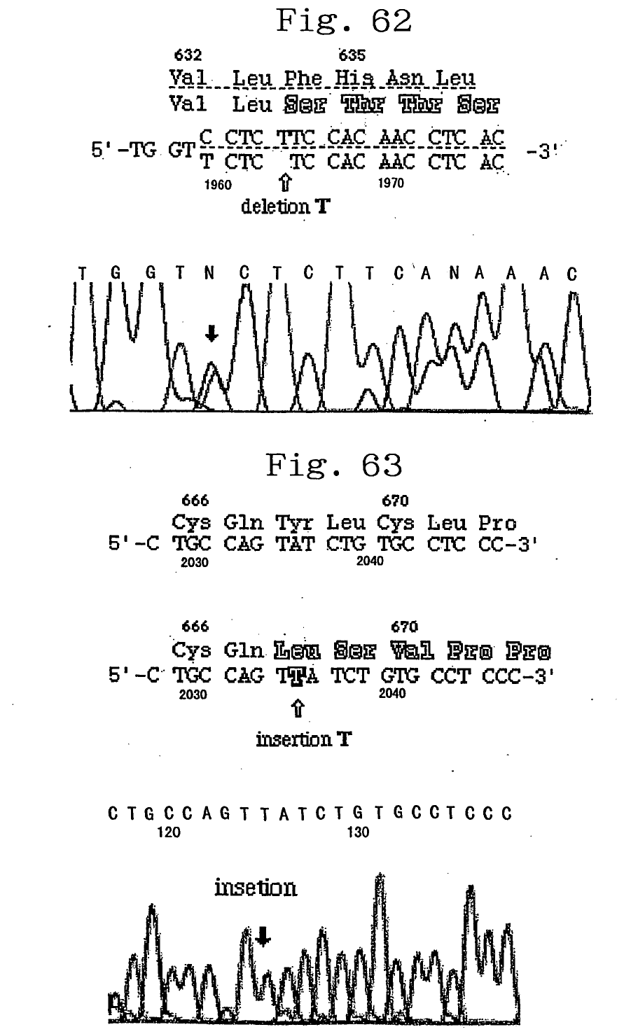

FH case 63 was found to have an insertion of a base (T) at nucleotide 2035 in exon 14 of the LDL-R gene, producing a stop codon at the position counting 38 downstream of the codon of the site of insertion (FIG. 63). This insertion mutation anticipates production of an abnormal LDL-R protein consisting of 694 amino acid residues.

Thus, the results have clarified that a risk factor indicating abnormality in lipid metabolism can be detected through correlation between the insertion mutation occurring at a position corresponding to nucleotide 2035 of the LDL-R gene and abnormality in lipid metabolism.

FH case 64 was found to have a substitution C→T at nucleotide 2054 (C) in exon 14 of the LDL-R gene, which resulted in a mutation of the LDL-R protein such that the amino acid residue Pro encoded by-the 664th codon was replaced by Leu.

These results have clarified that a risk factor indicating abnormality in lipid metabolism can be detected through correlation between the gene mutation occurring at a site coding for amino acid residue 664, proline, of the LDL-R protein encoded by the LDL-R gene and abnormality in lipid metabolism.

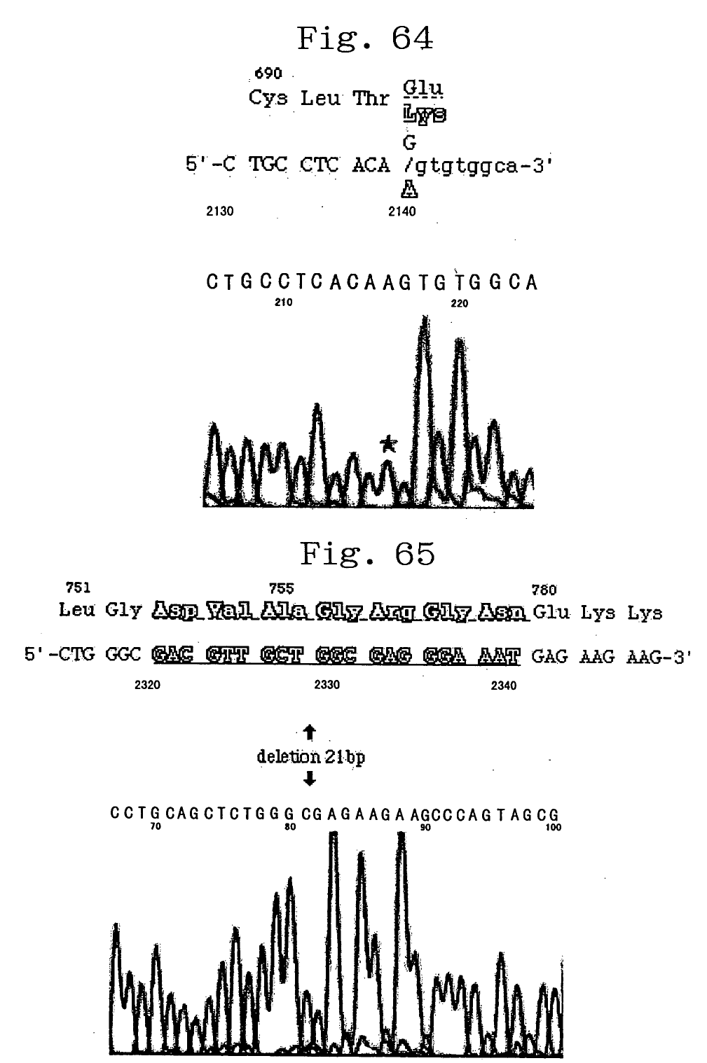

FH case 65 was found to have a substitution G→A at nucleotide 2140 (G) in exon 14 of the LDL-R gene, which resulted in a mutation of the LDL-R protein such that the amino acid residue Glu encoded by the 693rd codon was replaced by Lys (FIG. 64).

These results have clarified that a risk factor indicating abnormality in lipid metabolism can be detected through correlation between the gene mutation occurring at a site coding for amino acid residue 693, glutamic acid, of the LDL-R protein and abnormality in lipid metabolism.

FH case 66 was found to have a loss of 21 bases (GACGTTGCTGGCAGAGGAAAT) corresponding to nucleotides 2320 to 2340 in exon 16 of the LDL-R gene. This deletion did not cause any frame shift in codons for amino acid residues, and was a deletion mutation in which the 753rd to 759th codons composed of 21 nucleotides coding for 7 amino acid residues (Asp-Val-Ala-Gly-Arg-Gly-Asn) were deleted (FIG. 65). This deletion mutation anticipates production of an abnormal LDL-R protein composed of amino acid residues in a number 7 fewer than those constituting a native LDL-R protein.

Thus, the results have clarified that a risk factor indicating abnormality in lipid metabolism can be detected through correlation between the deletion mutation occurring in nucleotides 2320 to 2340 of the LDL-R gene and abnormality in lipid metabolism.

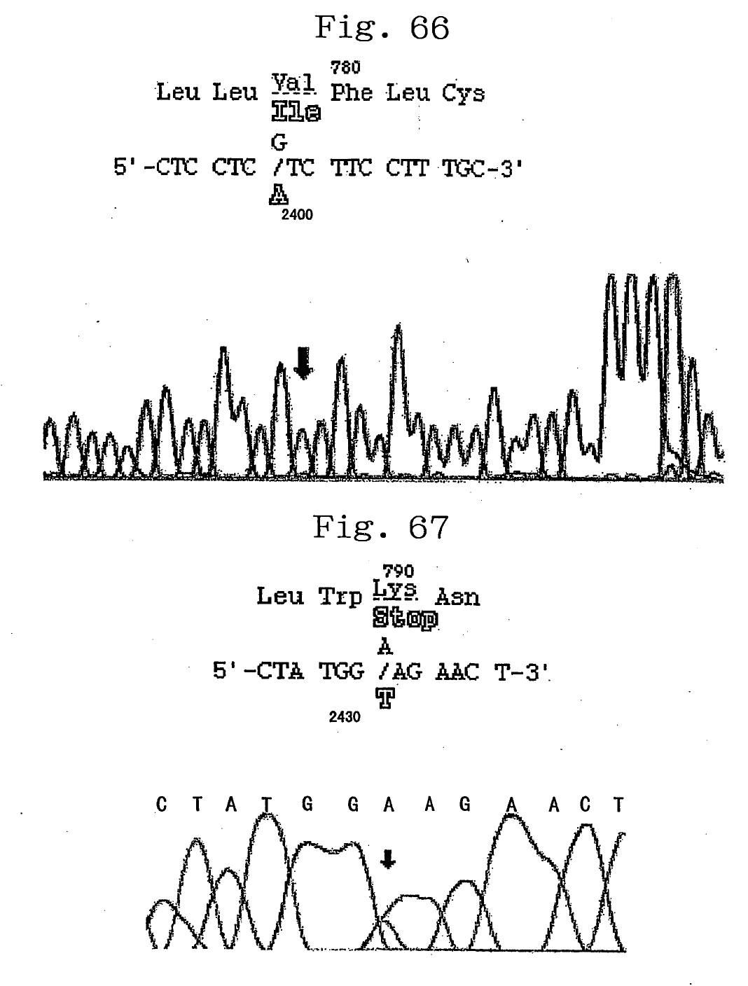

FH case 67 was found to have a substitution G→A at nucleotide 2398 (G) in exon 17 of the LDL-R gene, which resulted in a mutation of the LDL-R protein such that the amino acid residue encoded by the 779th codon, Val, was replaced by Ile (FIG. 66).

These results have clarified that a risk factor indicating abnormality in lipid metabolism can be detected through correlation between the gene mutation occurring at a site coding for amino acid residue 779, valine, of the LDL-R protein encoded by the LDL-R gene and abnormality in lipid metabolism.

FH case 68 was found to have a substitution A→T at nucleotide 2431 (A) in exon 17 of the LDL-R gene, which resulted in a mutation of the LDL-R protein wherein the 790th codon coding for the amino acid residue Lys was changed to a stop codon (FIG. 67). This base substitution anticipates production of an abnormal LDL-R protein consisting of 789 amino acid residues.

These results have clarified that a risk factor indicating abnormality in lipid metabolism can be detected through correlation between the gene mutation occurring at a site coding for amino acid residue 790, lysine, of the LDL-R protein encoded by the LDL-R gene and abnormality in lipid metabolism.

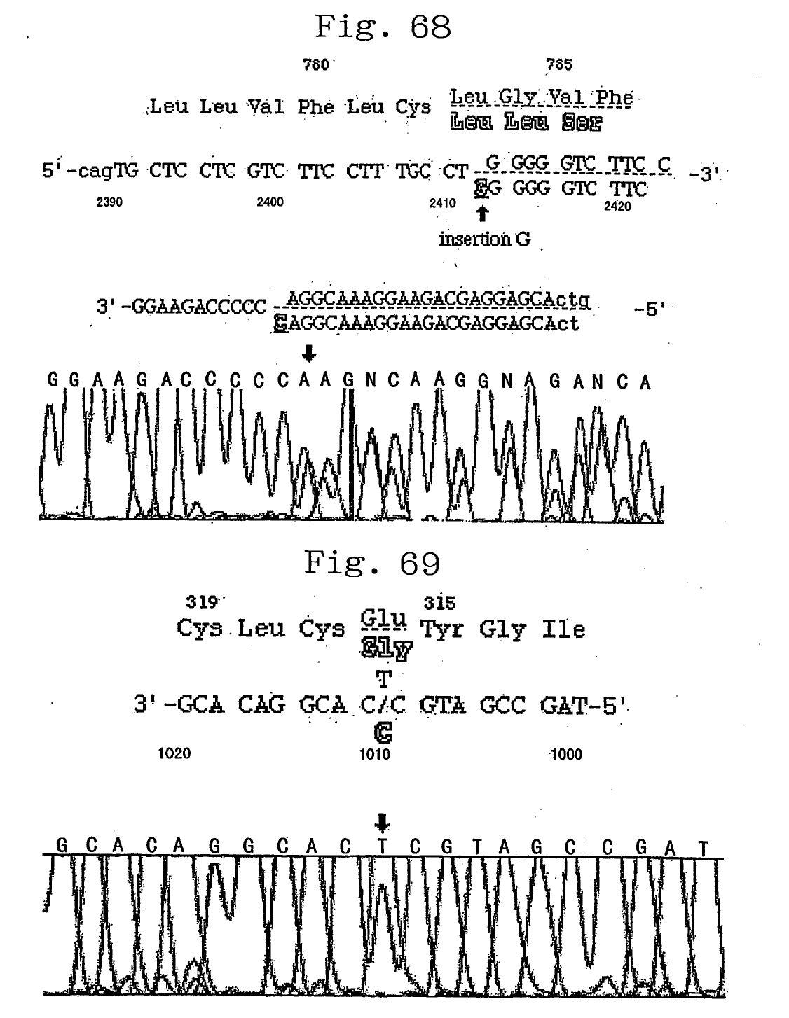

FH case 69 was found to have an insertion of a base (G) at nucleotide 2412 in exon 17 of the LDL-R gene, producing a stop codon at the position counting 13 downstream of the codon 783 of the site of insertion (FIG. 68). This insertion mutation anticipates production of an abnormal LDL-R protein consisting of 794 amino acid residues.

Thus, the results have clarified that a risk factor indicating abnormality in lipid metabolism can be detected through correlation between the insertion mutation occurring at a position corresponding to nucleotide 2412 of the LDL-R gene and abnormality in lipid metabolism.

FH case 70 was found to have a substitution C→T at nucleotide 2579 (C) in exon 18 of the LDL-R gene, which resulted in a mutation of the LDL-R protein such that the amino acid residue Ala encoded by the 829th codon was replaced by Val.

These results have clarified that a risk factor indicating abnormality in lipid metabolism can be detected through correlation between the gene mutation occurring at a site coding for amino acid residue 829, alanine, of the LDL-R protein encoded by the LDL-R gene and abnormality in lipid metabolism.

FH case 71 was found to have a substitution T→C at nucleotide 1010 (T) in exon 7 of the LDL-R gene, which resulted in a mutation of the LDL-R protein such that the amino acid residue Glu encoded by the 316th codon was replaced by Gly (FIG. 69).

These results have clarified that a risk factor indicating abnormality in lipid metabolism can be detected through correlation between the gene mutation occurring at a site coding for amino acid residue 316, glutamic acid, of the LDL-R protein encoded by the LDL-R gene and abnormality in lipid metabolism.

The above-described mutations of the LDL-R gene can be categorized into the following 5 groups: 1) a missense mutation; i.e., a base change that alters an amino acid; 2) a nonsense mutation; i.e., a base change that converts an amino acid to a stop codon; 3) a frame shift mutation; i.e., loss or gain of a nucleotide in a coding sequence of amino acids, to thereby shift a frame of translational codons, producing a stop codon on the downstream side; 4) an inframe mutation; i.e., loss or gain of a nucleotide in a coding sequence of amino acids, to thereby shift a frame of translational codons without producing a stop codon; and 5) a silent mutation; i.e., a base change that has no effect on amino acid sequence. The mutations of groups 1) to 4) result in a quantitatively or qualitatively abnormal LDL-R protein after synthesis, disabling the LDL-R protein in living organisms from exhibiting normal functions, and elevating the blood cholesterol level. A mutation of group 5) does not itself cause any quantitative or qualitative abnormalities of LDL-R protein, but in analysis, this mutation can be used as a risk factor that indicates a genetic polymorphism which may be related to an abnormal LDL-R gene (disease-associated gene mutation), so as to determine the level of risk of suffering a disease (arteriosclerosis or ischemic heart disease).

In relation to the analyses of the present study, the following polymorphisms of the LDL-R gene were identified as the mentioned disease-associated gene mutations:

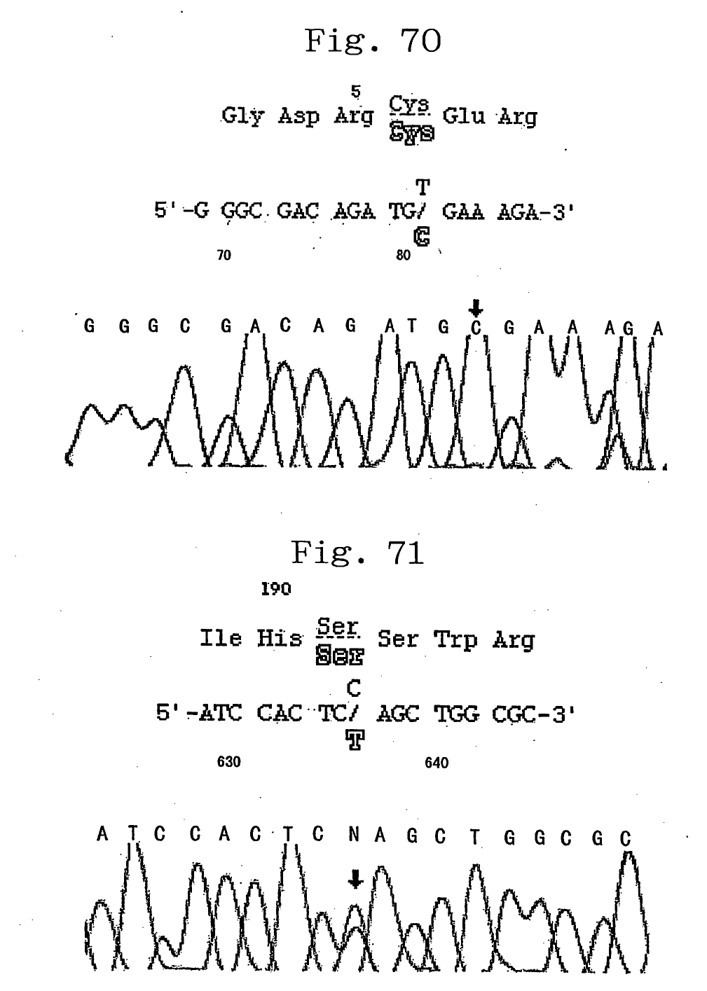

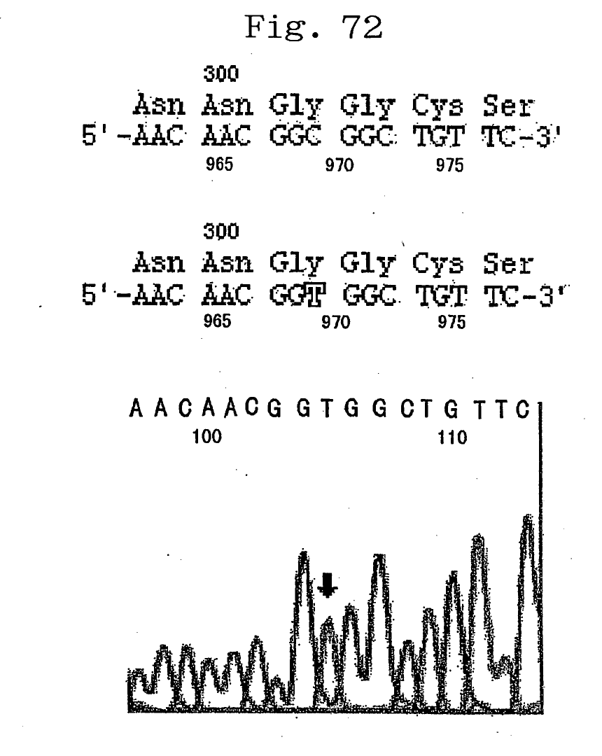

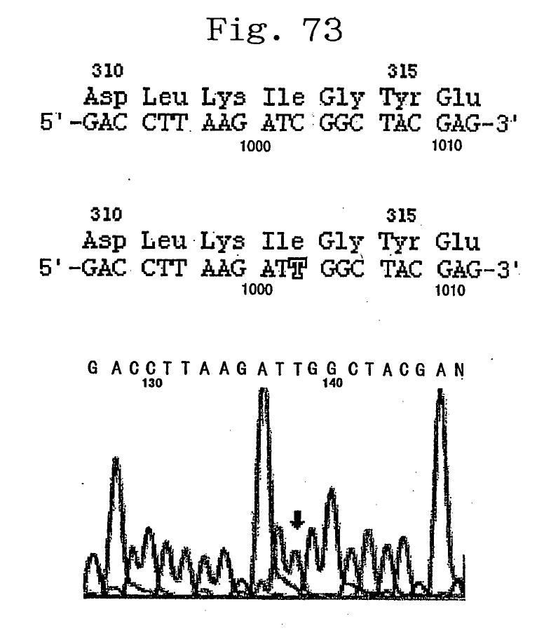

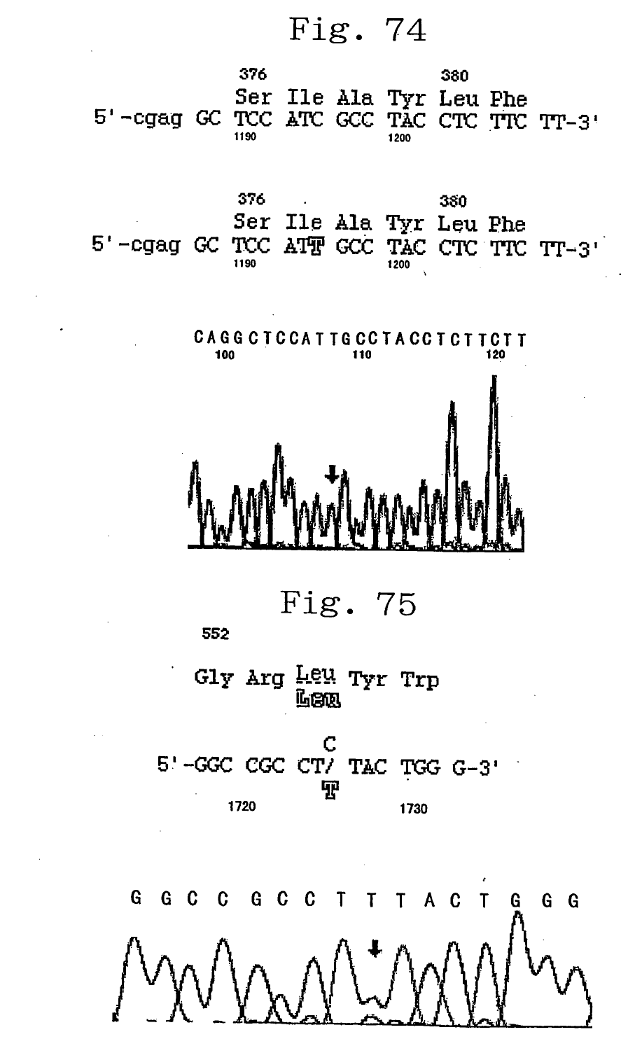

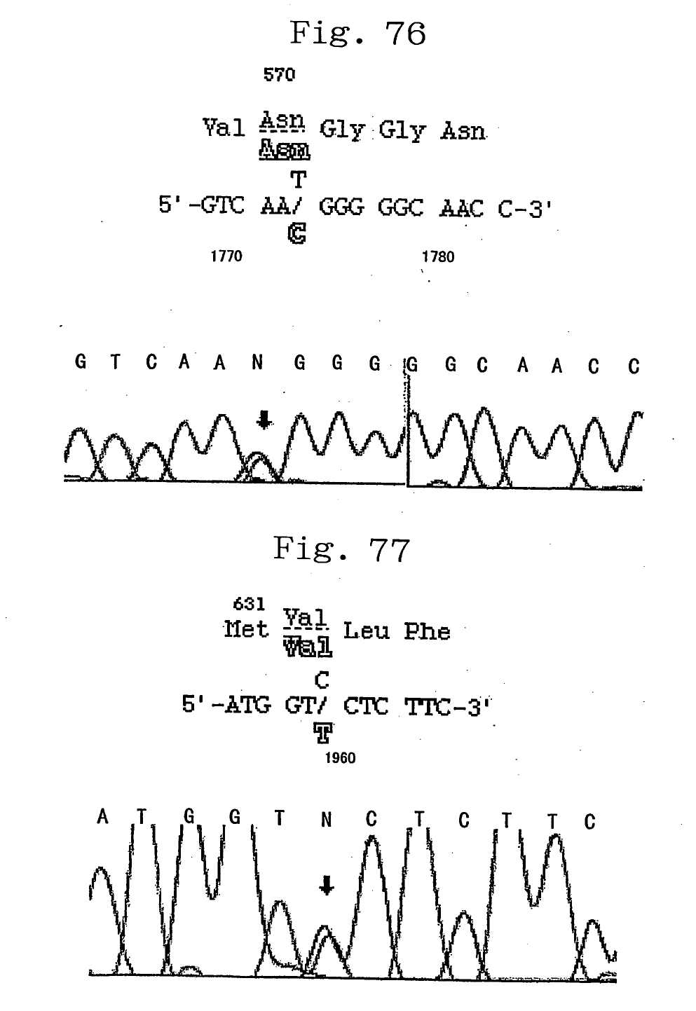

Cys6Cys in which the 81st base has been changed from T to C (FIG. 70), Ser191Ser in which the 636th base has been changed from C to T (FIG. 71), Gly301Gly in which the 969th base has been changed from C to T (FIG. 72), Ile313Ile in which the 1002nd base has been changed from C to T (FIG. 73), Ile377Ile in which the 1195th base has been changed from C to T (FIG. 74), Leu554Leu in which the 1725th base has been changed from C to T (FIG. 75), Asn570Asn in which the 1773rd base has been changed from T to C (FIG. 76), and Ala585Ala in which the 1817th base has been changed from C to T and Val632Val in which the 1959th base has been changed from C to T (FIG. 77).

### Industrial Applicability

The present invention contemplates provision of widely applicable means for attaining ensured diagnosis of familial hyperlipemia.

1. A method of detecting abnormality of lipid metabolism, which comprises a step of correlating abnormality in lipid metabolism with one or more gene mutations selected from the group consisting of the below-described 1 through 65 gene mutations, to thereby detect a risk factor concerning abnormality of lipid metabolism:
1) a mutation of a low-density lipoprotein receptor gene coding for low-density lipoprotein receptor protein, the mutation occurring at a site coding for amino acid residue 25, cysteine, of the low-density lipoprotein receptor protein;
2) a deletion mutation occurring in nucleotides 156 to 160 of the low-density lipoprotein receptor gene;
3) a mutation of the low-density lipoprotein receptor gene occurring at a site coding for amino acid residue 50, glycine, of the low-density lipoprotein receptor protein encoded by the gene;
4) a mutation of the low-density lipoprotein receptor gene occurring at a site coding for amino acid residue 74, cysteine, of the low-density lipoprotein receptor protein encoded by the gene;
5) a deletion mutation-occurring in nucleotide 314, cytosine, of the low-density lipoprotein receptor gene;
6) a mutation of the low-density lipoprotein receptor gene occurring at a site coding for amino acid residue 81, glutamine, of the low-density lipoprotein receptor protein encoded by the gene;
7) a mutation of the low-density lipoprotein receptor gene occurring at a site coding for amino acid residue 88, cysteine, of the low-density lipoprotein receptor protein encoded by the gene;
8) a mutation of the low-density lipoprotein receptor gene occurring at a site coding for amino acid residue 90, glutamine, of the low-density lipoprotein receptor protein encoded by the gene;
9) a mutation of the low-density lipoprotein receptor gene occurring at a site coding for amino acid residue 94, arginine, of the low-density lipoprotein receptor protein encoded by the gene;
10) a deletion mutation occurring in nucleotides 355 to 361, of the low-density lipoprotein receptor gene;
11) a mutation of the low-density lipoprotein receptor gene occurring at a site coding for amino acid residue 100, cysteine, of the low-density lipoprotein receptor protein encoded by the gene;.
12) a deletion mutation occurring in nucleotides 382 and 383, of the low-density lipoprotein receptor gene;
13) an insertion mutation occurring at a position of nucleotide 390 of the low-density lipoprotein receptor gene;
14) a mutation of the low-density lipoprotein receptor gene occurring at a site coding for amino acid residue 113, cysteine, of the low-density lipoprotein receptor protein encoded by the gene;
15) a mutation of the low-density lipoprotein receptor gene occurring at a site coding for amino acid residue 115, aspartic acid, of the low-density lipoprotein receptor protein encoded by the gene;
16) a mutation of the low-density lipoprotein receptor gene occurring at a site coding for amino acid residue 119, glutamic acid, of the low-density lipoprotein receptor protein encoded by the gene;
17) a deletion mutation occurring in nucleotides 578 to 584, of the low-density lipoprotein receptor gene;
18) an insertion mutation occurring at a position of nucleotide 682 of the low-density lipoprotein receptor gene;
19) a deletion mutation occurring in nucleotides 526 to 529, of the low-density lipoprotein receptor gene;
20) an insertion mutation occurring at a position of nucleotide 661 of the low-density lipoprotein receptor gene;
21) a mutation of the low-density lipoprotein receptor gene occurring at a site coding for amino acid residue 207, glutamic acid, of the low-density lipoprotein receptor protein encoded by the gene;
22) an insertion mutation occurring at a position of nucleotide 944 of the low-density lipoprotein receptor gene;
23) a deletion mutation occurring in nucleotide 948, cytosine, of the low-density lipoprotein receptor gene;
24) a mutation of the low-density lipoprotein receptor gene occurring at a site coding for amino acid residue 317, cysteine, of the low-density lipoprotein receptor protein encoded by the gene;
25) a deletion mutation occurring in nucleotides 1114 to 1134, of the low-density lipoprotein receptor gene;
26) an insertion mutation occurring at a position of nucleotide 1062 of the low-density lipoprotein receptor gene;
27) a mutation of the low-density lipoprotein receptor gene occurring at a site coding for amino acid residue 336, glutamic acid, of the low-density lipoprotein receptor protein encoded by the gene;
28) a mutation of the low-density lipoprotein receptor gene occurring at a site coding for amino acid residue 337, cysteine, of the low-density lipoprotein receptor protein encoded by the gene;
29) a mutation of the low-density lipoprotein receptor gene occurring at a site coding for amino acid residue 356, cysteine, of the low-density lipoprotein receptor protein encoded by the gene;
30) a mutation of the low-density lipoprotein receptor gene occurring at a site coding for amino acid residues 351 to 354, glutamic acid—glycine—glycine—tyrosine, of the low-density lipoprotein receptor protein encoded by the gene;
31) a mutation of the low-density lipoprotein receptor gene occurring at a site coding for amino acid residue 358, cysteine, of the low-density lipoprotein receptor protein encoded by the gene;
32) a mutation occurring at nucleotide −10 (guanine) in the 5′-end-side acceptor region in intron 8 of the low-density lipoprotein receptor gene;
33) a mutation of the low-density lipoprotein receptor gene occurring at a site coding for amino acid residue 382, phenylalanine, of the low-density lipoprotein receptor protein encoded by the gene;
34) a mutation occurring in nucleotide 1599, of the low-density lipoprotein receptor gene;
35) a deletion mutation occurring in nucleotides 1202 to 1204, of the low-density lipoprotein receptor gene;
36) a mutation of the low-density lipoprotein receptor gene occurring at a site coding for amino acid residue 385, arginine, of the low-density lipoprotein receptor protein encoded by the gene;
37) a mutation of the low-density lipoprotein receptor gene occurring at a site coding for amino acid residue 387, glutamic acid, of the low-density lipoprotein receptor protein encoded by the gene;
38) an insertion mutation occurring at a position of nucleotide 1242 of the low-density lipoprotein receptor gene;
39) a mutation of the low-density lipoprotein receptor gene occurring at a site coding for amino acid residue 401, leucine, of the low-density lipoprotein receptor protein encoded by the gene;
40) a mutation of the low-density lipoprotein receptor gene occurring at a site coding for amino acid residue 410, alanine, of the low-density lipoprotein receptor protein encoded by the gene;
41) a mutation of the low-density lipoprotein receptor gene occurring at a site coding for amino acid residue 412, aspartic acid, of the low-density lipoprotein receptor protein encoded by the gene;
42) a mutation of the low-density lipoprotein receptor gene occurring at a site coding for amino acid residue 512, tryptophan, of the low-density lipoprotein receptor protein encoded by the gene;
43) a deletion mutation occurring in nucleotides 1652 to 1662, of the low-density lipoprotein receptor gene;
44) a deletion mutation occurring in nucleotide 1655, thymine, of the low-density lipoprotein receptor gene;. 45) an insertion mutation occurring at a position of nucleotide 1687 of the low-density lipoprotein receptor gene;
46) a mutation of the low-density lipoprotein receptor gene occurring at a site coding for amino acid residue 547, leucine, of the low-density lipoprotein receptor protein encoded by the gene;
47) a mutation from guanine to another base, occurring at nucleotide +1 (guanine) in a splice donor site of intron 11 starting from the nucleotide that is one base downstream the nucleotide 1705 of the low-density lipoprotein receptor protein gene;
48) a mutation from thymine to another base, occurring at nucleotide +2 (thymine) in a splice donor site of intron 12 starting from the nucleotide that is one base downstream the nucleotide 1845 of the low-density lipoprotein receptor protein gene;
49) a mutation of the low-density lipoprotein receptor gene occurring at a site coding for amino acid residue 556, tryptophan, of the low-density lipoprotein receptor protein encoded by the gene;
50) a mutation of the low-density lipoprotein receptor gene occurring at a site coding for amino acid residue 570, asparagine, of the low-density lipoprotein receptor protein encoded by the gene;
51) an insertion mutation occurring at a position of nucleotide 1779 of the low-density lipoprotein receptor gene;
52) a mutation of the low-density lipoprotein receptor gene occurring at a site coding for amino acid residue 587, proline, of the low-density lipoprotein receptor protein encoded by the gene;
53) a mutation of the low-density lipoprotein receptor gene occurring at a site coding for amino acid residue 591, alanine, of the low-density lipoprotein receptor protein encoded by the gene;
54) a deletion mutation occurring in nucleotides 1870 to 1872, of the low-density lipoprotein receptor gene;
55) a mutation of the low-density lipoprotein receptor gene occurring at a site coding for amino acid residue 612, arginine, of the low-density lipoprotein receptor protein encoded by the gene;
56) a deletion mutation occurring in nucleotide 1963, of the low-density lipoprotein receptor gene;
57) an insertion mutation occurring at a position of nucleotide 2035 of the low-density lipoprotein receptor gene;
58) a mutation of the low-density lipoprotein receptor gene occurring at a site coding for amino acid residue 664, proline, of the low-density lipoprotein receptor protein encoded by the gene;
59) a mutation of the low-density lipoprotein receptor gene occurring at a site coding for amino acid residue 693, glutamic acid, of the low-density lipoprotein receptor protein encoded by the gene;
60) a deletion mutation occurring in nucleotides 2320 to 2340, of the low-density lipoprotein receptor gene;
61) a mutation of the low-density lipoprotein receptor gene occurring at a site coding for amino acid residue 779, valine, of the low-density lipoprotein receptor protein encoded by the gene;
62) a mutation of the low-density lipoprotein receptor gene occurring at a site coding for amino acid residue 790, lysine, of the low-density lipoprotein receptor protein encoded by the gene;
63) an insertion mutation occurring at a position of nucleotide 2412 of the low-density lipoprotein receptor gene;
64) a mutation of the low-density lipoprotein receptor gene occurring at a site coding for amino acid residue 829, alanine, of the low-density lipoprotein receptor protein encoded by the gene; and
65) a mutation of the low-density lipoprotein receptor gene occurring at a site coding for amino acid residue 316, glutamic acid, of the low-density lipoprotein receptor protein encoded by the gene.

2. The method of detecting abnormality of lipid metabolism as recited in claim 1, wherein said one or more gene mutations selected from said group include at least one of the following gene mutations:
1) a mutation of a low-density lipoprotein receptor gene coding for low-density lipoprotein receptor protein, the mutation occurring at a site coding for amino acid residue 25, cysteine, of the low-density lipoprotein receptor protein;
2) a deletion mutation occurring in nucleotides 156 to 160 of the low-density lipoprotein receptor gene;
3) a mutation of the low-density lipoprotein receptor gene occurring at a site coding for amino acid residue 50, glycine, of the low-density lipoprotein receptor protein encoded by the gene;
4) a mutation of the low-density lipoprotein receptor gene occurring at a site coding for amino acid residue 74, cysteine, of the low-density lipoprotein receptor protein encoded by the gene;
5) a deletion mutation occurring in nucleotide 314, cytosine, of the low-density lipoprotein receptor gene;
7) a mutation of the low-density lipoprotein receptor gene occurring at a site coding for amino acid residue 88, cysteine, of the low-density lipoprotein receptor protein encoded by the gene;
8) a mutation of the low-density lipoprotein receptor gene occurring at a site coding for amino acid residue 90, glutamine, of the low-density lipoprotein receptor protein encoded by the gene;
9) a mutation of the low-density lipoprotein receptor gene occurring at a site coding for amino acid residue 94, arginine, of the low-density lipoprotein receptor protein encoded by the gene;
10) a deletion mutation occurring in nucleotides 355 to 361, of the low-density lipoprotein receptor gene;
11) a mutation of the low-density lipoprotein receptor gene occurring at a site coding for amino acid residue 100, cysteine, of the low-density lipoprotein receptor protein encoded by the gene;
12) a deletion mutation occurring in nucleotides 382 and 383, of the low-density lipoprotein receptor gene;
13) an insertion mutation occurring at a position of nucleotide 390 of the low-density lipoprotein receptor gene;
14) a mutation of the low-density lipoprotein receptor gene occurring at a site coding for amino acid residue 113, cysteine, of the low-density lipoprotein receptor protein encoded by the gene;
15) a mutation of the low-density lipoprotein receptor gene occurring at a site coding for amino acid residue 115, aspartic acid, of the low-density lipoprotein receptor protein encoded by the gene;
17) a deletion mutation occurring in nucleotides 578 to 584, of the low-density lipoprotein receptor gene;
18) an insertion mutation occurring at a position of nucleotide 682 of the low-density lipoprotein receptor gene;
19) a deletion mutation occurring in nucleotides 526 to 529, of the low-density lipoprotein receptor gene;
22) an insertion mutation occurring at a position of nucleotide 944 of the low-density lipoprotein receptor gene;
23) a deletion mutation occurring in nucleotide 948, cytosine, of the low-density lipoprotein receptor gene;
24) a mutation of the low-density lipoprotein receptor gene occurring at a site coding for amino acid residue 317, cysteine, of the low-density lipoprotein receptor protein encoded by the gene;
25) a deletion mutation occurring in nucleotides 1114 to 1134, of the low-density lipoprotein receptor gene;
26) an insertion mutation occurring at a position of nucleotide 1062 of the low-density lipoprotein receptor gene;
27) a mutation of the low-density lipoprotein receptor gene occurring at a site coding for amino acid residue 336, glutamic acid, of the low-density lipoprotein receptor protein encoded by the gene;
28) a mutation of the low-density lipoprotein receptor gene occurring at a site coding for amino acid residue 337, cysteine, of the low-density lipoprotein receptor protein encoded by the gene;
29) a mutation of the low-density lipoprotein receptor gene occurring at a site coding for amino acid residue 356, cysteine, of the low-density lipoprotein receptor protein encoded by the gene;
32) a mutation occurring at nucleotide −10 (guanine) in the 5′-end-side acceptor region in intron 8 of the low-density lipoprotein receptor gene;
34) a mutation occurring in nucleotide 1599, of the low-density lipoprotein receptor gene;
35) a deletion mutation occurring in nucleotides 1202 to 1204, of the low-density lipoprotein receptor gene;
39) a mutation of the low-density lipoprotein receptor gene occurring at a site coding for amino acid residue 401, leucine, of the low-density lipoprotein receptor protein encoded by the gene;
43) a deletion mutation occurring in nucleotides 1652 to 1662, of the low-density lipoprotein receptor gene;
44) a deletion mutation occurring in nucleotide 1655, thymine, of the low-density lipoprotein receptor gene;
47) a mutation from guanine to another base, occurring at nucleotide +1 (guanine) in a splice donor site of intron 11 starting from the nucleotide that is one base downstream the nucleotide 1705 of the low-density lipoprotein receptor protein gene;
49) a mutation of the low-density lipoprotein receptor gene occurring at a site coding for amino acid residue 556, tryptophan, of the low-density lipoprotein receptor protein encoded by the gene;
50) a mutation of the low-density lipoprotein receptor gene occurring at a site coding for amino acid residue 570, asparagine, of the low-density lipoprotein receptor protein encoded by the gene;
51) an insertion mutation occurring at a position of nucleotide 1779 of the low-density lipoprotein receptor gene;
53) a mutation of the low-density lipoprotein receptor gene occurring at a site coding for amino acid residue 591, alanine, of the low-density lipoprotein receptor protein encoded by the gene;
54) a deletion mutation occurring in nucleotides 1870 to 1872, of the low-density lipoprotein receptor gene;
55) a mutation of the low-density lipoprotein receptor gene occurring at a site coding for amino acid residue 612, arginine, of the low-density lipoprotein receptor protein encoded by the gene;
56) a deletion mutation occurring in nucleotide 1963, of the low-density lipoprotein receptor gene;
57) an insertion mutation occurring at a position of nucleotide 2035 of the low-density lipoprotein receptor gene;
60) a deletion mutation occurring in nucleotides 2320 to 2340, of the low-density lipoprotein receptor gene;
61) a mutation of the low-density lipoprotein receptor gene occurring at a site coding for amino acid residue 779, valine, of the low-density lipoprotein receptor protein encoded by the gene;
63) an insertion mutation occurring at a position of nucleotide 2412 of the low-density lipoprotein receptor gene;
64) a mutation of the low-density lipoprotein receptor gene occurring at a site coding for amino acid residue 829, alanine, of the low-density lipoprotein receptor protein encoded by the gene; and
65) a mutation of the low-density lipoprotein receptor gene occurring at a site coding for amino acid residue 316, glutamic acid, of the low-density lipoprotein receptor protein encoded by the gene.

3. A method of detecting a disease, comprising detecting a risk factor for arteriosclerosis and/or ischemic heart disease through employment, as an index, the abnormality in lipid metabolism detected by the method for detecting abnormality of lipid metabolism as recited in claim 1 or 2.
